(19) United States
(12) Patent Application Publication
(10) Pub. No.: US 2026/0159507 A1
(43) Pub. Date: Jun. 11, 2026

(54) GLYCOSYLATION AND ALKYLATION METHOD INVOLVING BENZIODOXOLE REAGENT

(71) Applicant: HUAZHONG UNIVERSITY OF SCIENCE AND TECHNOLOGY, Hubei (CN)

(72) Inventors: Qian WAN, Hubei (CN); Lingkui MENG, Hubei (CN); Jing ZENG, Hubei (CN); Yulin TAN, Hubei (CN); Zhirou Chen, Hubei (CN); Zhiwen Liao, Hubei (CN); Zhihua Liang, Hubei (CN); Yue Li, Hubei (CN)

(73) Assignee: HUAZHONG UNIVERSITY OF SCIENCE AND TECHNOLOGY, Hubei (CN)

(21) Appl. No.: 19/348,741

(22) Filed: Oct. 2, 2025

(30) Foreign Application Priority Data
Dec. 6, 2024 (CN) 202411796701.0

(51) Int. Cl. C07D 407/12; C07C 13/66; C07C 61/29; C07C 235/06; C07D 309/08; C07D 347/00; C07D 405/04; C07D 407/04; C07D 407/08; C07D 407/14; C07D 421/12

(52) U.S. Cl. CPC C07D 407/12; C07C 13/66; C07C 61/29; C07C 235/06; C07D 309/08; C07D 347/00; C07D 405/04; C07D 407/04; C07D 407/08; C07D 407/14; C07D 421/12

(57) ABSTRACT

The disclosure belongs to the technical field of organic synthesis and discloses a glycosylation and alkylation method involving a benziodoxole reagent. In the glycosylation method or alkylation method, a raw material (glycosyl donor or alkylation reagent) under a combined action of the benziodoxole reagent and an acid catalyst are activated and then reacted with a nucleophilic reagent to obtain a corresponding glycosylation product or alkylation product. In the method of the disclosure, by introducing the benziodoxole reagent and working in combination with an acid catalyst, efficient reaction is achieved without the need to use a precious metal heavy metal catalyst.

### CROSS-REFERENCE TO RELATED APPLICATION

This application claims the priority benefit of China application serial no. 202411796701.0, filed on Dec. 6, 2024. The entirety of the above-mentioned patent application is hereby incorporated by reference herein and made a part of this specification.

### BACKGROUND

### Technical Field

The disclosure belongs to the technical field related to organic synthesis, and more specifically, relates to a glycosylation and alkylation method involving a benziodoxole reagent.

### Description of Related Art

Hypervalent iodine reagents possess rich and diverse chemical reactivity and are extensively applied in organic synthesis due to their unique properties. Hypervalent iodine reagents are environmentally friendly and possess properties similar to metals. Hypervalent iodine reagents have been show their ability to replace polluting metal reagents in many reactions, thereby aligning with green chemistry principles. This makes them highly valuable for modern chemical synthesis.

Benziodoxole reagents have been developed in many varieties and have found extensive applications in organic synthesis, such as serving as intermediates for synthesizing certain compounds, acting as trifluoromethylation reagents, dearomatization, construction of carbon-heteroatom bonds, and other applications. These applications demonstrate the tremendous potential of this class of reagents. In addition, benziodoxole reagents may also be applied in carbohydrate synthesis and alkylation reactions, so the application scope of benziodoxole reagents is further expanded.

Glycosylation and alkylation reactions are both extremely important types of reactions in organic chemistry. Existing glycosylation methods often suffer from issues such as the generation of highly reactive by-products, acid accumulation, or the requirement for precious metal catalysts. Meanwhile, conventional alkylation methods also present challenges, typically requiring strongly basic conditions and exhibiting poor compatibility with ester protecting groups. The development of novel glycosylation and alkylation methods is crucial for enabling more efficient and mild syntheses of complex molecules.

### SUMMARY

In response to the above limitations or needs for improvement in existing technologies, the present invention provides glycosylation and alkylation methods employing benziodoxole reagents. These methods involve the introduction of a benziodoxole reagent, which acts in concert with an acid catalyst to facilitate the reaction between glycosyl donor or alkylation reagents and nucleophiles, yielding the corresponding glycosylation or alkylation products. Simultaneously, stable iodine (III) salts or disulfide with an o-iodobenzyl alcohol byproduct are generated. Thereby, the methods address issues associated with conventional glycosylation reactions, such as the production of highly reactive byproducts, acid accumulation, or the need for precious metal catalysts, as well as circumvent the requirement for basic conditions in traditional alkylation methods.

To achieve the above objective, according to one aspect of the disclosure, a glycosylation method involving a benziodoxole reagent is provided, the method specifically includes the following steps. A glycosyl donor as shown in formula I under a combined action of the benziodoxole reagent having a structural formula as shown in formula V and an acid catalyst are activated and then reacted with a nucleophilic reagent to obtain a glycosylation product as shown in formula VI, with a reaction route as shown in formula 1:

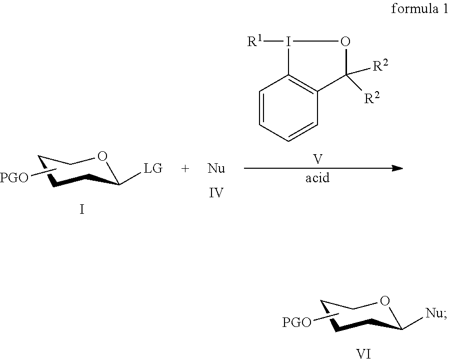

In the glycosyl donor represented by formula I, one or a plurality of hydroxyl groups on a sugar ring are protected by a glycosyl protecting group, and LG is an alkylthio group, an arylthio group, an aryl selenoyl group, a thiocarboxylate group, an o-alkynyl benzoate group, a pentenoate group, a pentynoate group, or a hexynoate group.

In the benziodoxole reagent having a structural formula as shown in formula V, R¹ is chlorine, hydroxyl, trimethylsilyloxy, trifluoromethanesulfonyl ester, or benziodooxolaneoxy, and R² is methyl or trifluoromethyl.

Nu in formula 1 represents the nucleophilic reagent, and the nucleophilic reagent is selected from primary alcohol, secondary alcohol, or carboxylic acid.

Preferably, the glycosyl donor represented by formula I is selected from compounds having any one of the following structures:

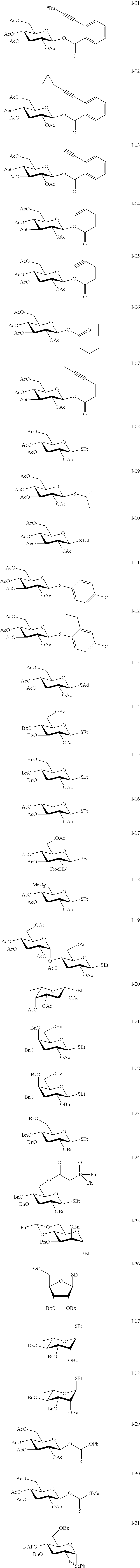

Preferably, the benziodoxole reagent having the structure shown in formula V is selected from compounds having any one of the following structures:

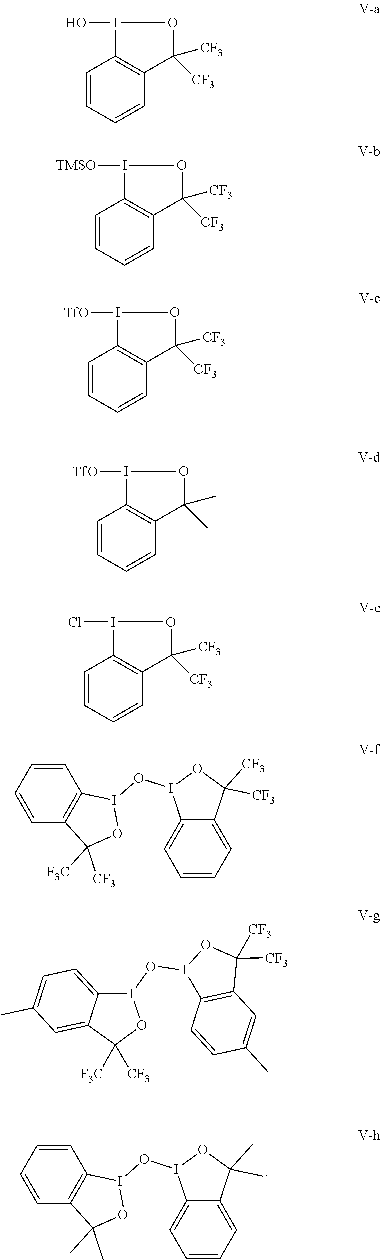

Preferably, a molar ratio of the glycosyl donor to the benziodoxole reagent is 1:(0.25 to 0.5).

Preferably, the nucleophilic reagent is selected from compounds having any one of the following structures:

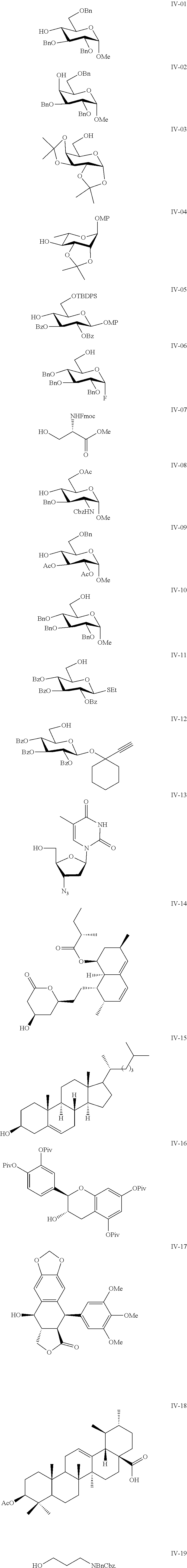

According to another aspect of the disclosure, an alkylation method involving a benziodoxole reagent is provided, the method specifically includes the following steps. An alkylation reagent as shown in formula II under a combined action of the benziodoxole reagent having a structure as shown in formula V and an acid catalyst are activated and then reacted with a nucleophilic reagent to obtain an alkylation product as shown in formula VII, with a reaction route as shown in formula 2. Alternatively, an alkylation reagent as shown in formula III under the combined action of the benziodoxole reagent having a structure as shown in formula V and an acid catalyst are activated and then reacted with a nucleophilic reagent to obtain an alkylation product as shown in formula VIII, with a reaction route as shown in formula 3:

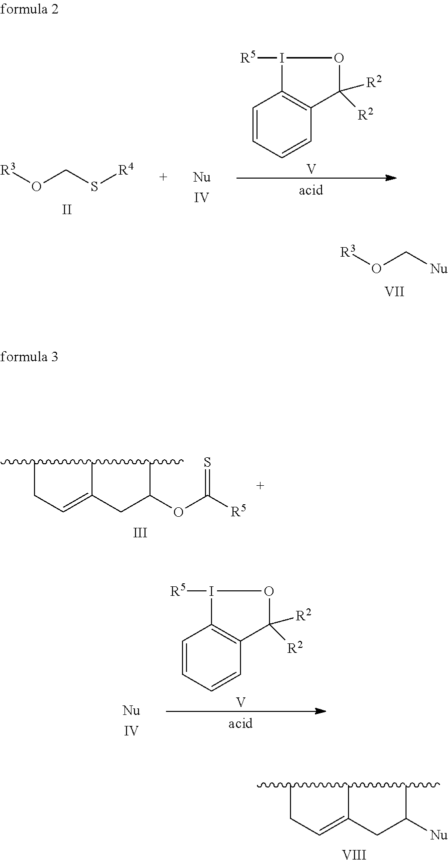

The alkylation reagent represented by formula II is a thioether alkylation reagent, where R³ is an alkyl group, and R⁴ is an alkyl group or an aryl group,

The alkylation reagent represented by formula III is a steroid derivative containing a high allyl thiocarbonate structure, where the steroid is cholesterol, diosgenin, dehydroepiandrosterone, pregnenolone, β-sitosterol, or stigmasterol, wherein R⁵ is a phenoxy group or a methylthio group,

In the benziodoxole reagent having a structural formula as shown in formula V, R¹ is chlorine, hydroxyl, trimethylsilyloxy, trifluoromethanesulfonyl ester, or benziodooxolaneoxy, and R² is methyl or trifluoromethyl.

Nu in both formula II and formula III represents the nucleophilic reagent, and the nucleophilic reagent is selected from primary alcohol, secondary alcohol, phenol, or phosphoric acid.

Preferably, the alkylation reagent represented by formula II is selected from compounds having any one of the following structures:

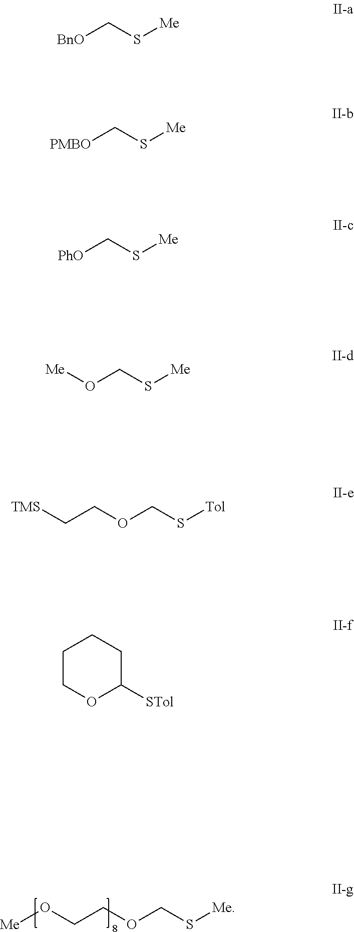

The alkylation reagent represented by formula III is selected from compounds having any one of the following structures:

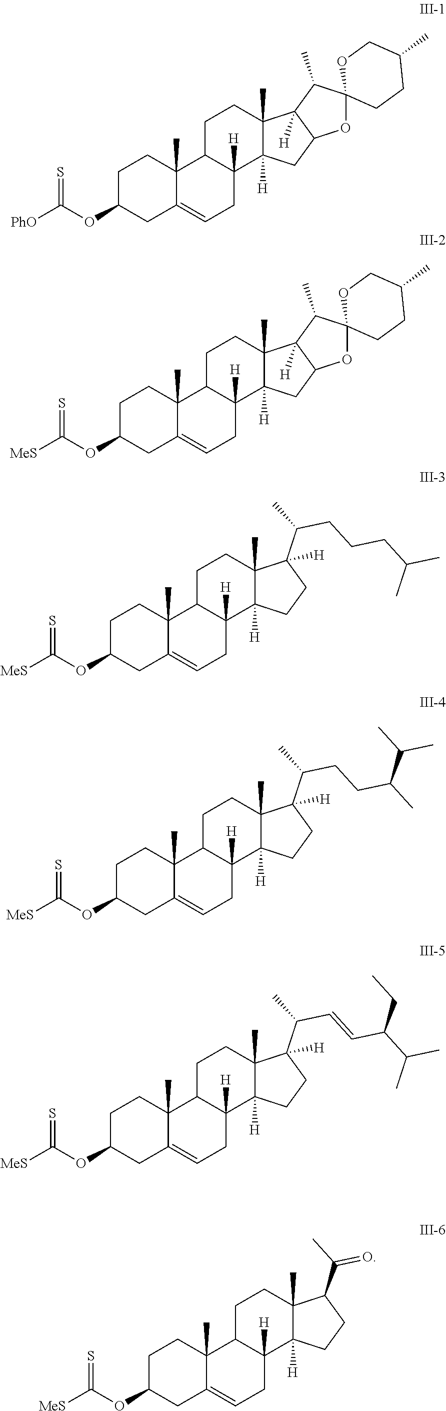

Preferably, the benziodoxole reagent having the structure shown in formula V is selected from compounds having any one of the following structures:

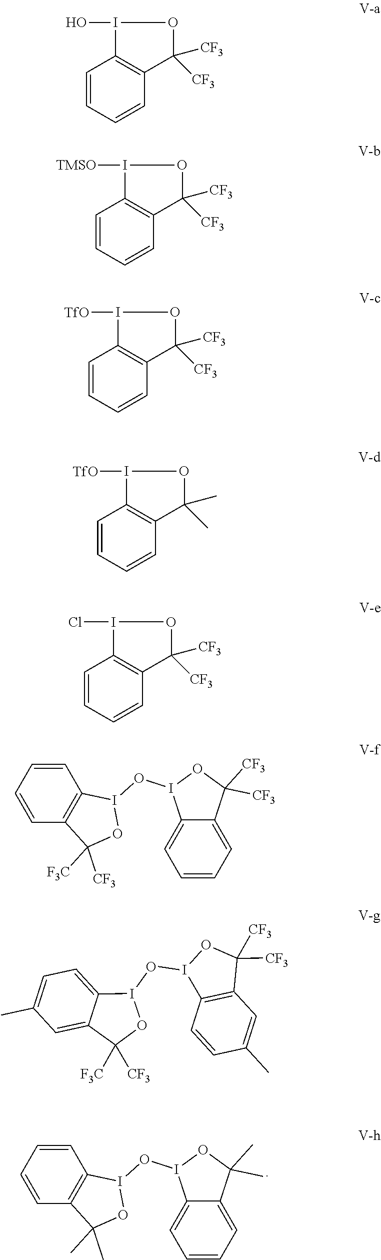

Preferably, a molar ratio of the alkylation reagent represented by formula II to the benziodoxole reagent is 1:(0.25 to 0.5). A molar ratio of the alkylation reagent represented by formula III to the benziodoxole reagent is 1:(0.25 to 0.5).

Preferably, the nucleophilic reagent is selected from compounds having any one of the following structures:

[chemical structure]

To sum up, the above technical solutions provided by the disclosure have the following beneficial effects compared with the related art.

(1) In the method of the disclosure, by introducing the benziodoxole reagent and working in combination with an acid catalyst, efficient reaction is achieved without the need to use a precious metal heavy metal catalyst. At the same time, in the glycosylation reaction, by merely introducing the substoichiometric amount of the benziodoxole reagent to oxidize the leaving group at the anomeric position of the sugar ring, followed by acid treatment, the glycosylation product is obtained in excellent yield. In the alkylation reaction, by introducing the substoichiometric amount of the benziodoxole reagent and further utilizing the acid catalysis, particularly for the compound with the structures such as those shown in formula IV-(20 to 26) and IV-30, efficient alkylation modification of hydroxyl groups is achieved, and the decomposition problems of certain compounds under strong acid or strong base conditions are avoided.

(2) The benziodoxole reagent used in the disclosure is easy to prepare in large quantities and has low cost.

(3) The disclosure uses a wide range of acids, which are commercially available common Brønsted acids or Lewis acids, providing flexible choices for the glycosylation of different substrates.

(4) The donor used in the disclosure may be thioglycosyle and ester glycosyle donors that have mature synthetic techniques, stable properties, and are easy to prepare and preserve.

To sum up, in the benziodiazole reagent-promoted glycosylation method provided by the disclosure, the problems existing in current methods such as expensive heavy metals, complex donor structures that are difficult to prepare, and poor donor stability are effectively overcome, so an easily implementable new solution for the synthesis of carbohydrate substances and alkylation reactions is provided.

### DESCRIPTION OF THE EMBODIMENTS

In order to make the objectives, technical solutions, and advantages of the disclosure clearer and more comprehensible, the disclosure is further described in detail with reference to the drawings and embodiments. It should be understood that the specific embodiments described herein serve to explain the disclosure merely and are not used to limit the disclosure. In addition, the technical features involved in the various embodiments of the invention described below can be combined with each other as long as the technical features do not conflict with each other.

Unless otherwise specified, the raw materials or reagents used in the disclosure are commercially available. All reagents are commercial grade and used as received according to standards. All moisture-sensitive reactions are conducted under argon atmosphere. Reactions are monitored by thin layer chromatography (TLC), detected by ultraviolet absorption (254 nm), and when necessary, sprayed with 10% by volume sulfuric acid ethanol solution and carbonized for color development at 80° C. to 150° C. Flash column chromatography employs silica gel H. ¹H and ¹³C nuclear magnetic resonance spectra are recorded on Bruker AV 400 and Bruker Av 600 using deuterated chloroform (CDCl₃) as the deuterated reagent. Chemical shifts (8) are expressed in ppm with tetramethylsilane as internal standard. Coupling constants (J) are expressed in Hz.

Unless otherwise specified, the scientific and technical terms and abbreviations used in the disclosure have the meanings commonly understood by a person having ordinary skill in the art. For instance, “equivalent” in the disclosure refers to the ratio of the amounts of substances participating in the reaction. For instance, the meanings of some abbreviations are as follows:

| Abbrev. | meaning | Abbrev. | meaning |
|---|---|---|---|
| Me | Methyl | Cbz | Benzyloxycarbonyl |
| Et | Ethyl | Ph | Phenyl |
| ⁱPr | Isopropyl | Piv | Pivaloyl |
| Ac | Acetyl | n-Bu | n-Butyl |
| Bn | Benzyl | Nap | Naphthylmethyl |
| Bz | Benzoyl | TMS | Trimethylsilyl |
| MP | p-Methoxyphenyl | Tol | p-Tolyl |
| TBDPS | tert-Butyldiphenylsilyl | Tf | Trifluoromethanesulfonyl |
| Fmoc | Fmoc | Ad | Adamantyl |
| PGO | Protecting group | Troc | Trichloroethoxycarbonyl |
| TMSOTf | Fluoromethanesulfonic acid | BF₃•Et₂O | Boron trifluoride etherate |
| | trimethylsilyl ester | | |
| TMSNTf₂ | N- | AgOTf | Silver |
| | (Trimethylsilyl)bis(trifluoromethane | | trifluoromethanesulfonate |
| | sulfonyl)imide | | |
| Nu | Nucleophilic reagent | | |

In the disclosure, a trivalent iodine reagent as shown in formula-II is prepared by the following exemplary method:

In step S1, an ortho-iodobenzyl alcohol compound as shown in formula I and periodic acid or periodate are placed in a reaction vessel, and an acetic acid aqueous solution is injected to obtain a reaction raw material mixture system.

In step S2, the reaction raw material mixture system of step S1 is stirred and reacted for 4 to 12 hours under heating conditions (110° C.).

In step S3, after the reaction in step S2 is completed, the reaction system is directly filtered after cooling, the filter cake is washed sequentially with water, acetonitrile, and dichloromethane to obtain part of a target product. A mother liquor is concentrated and slurried to continue obtaining part of the target product. The target products from both parts are combined to obtain the entire target product of the final reaction, namely a benziodoxole reagent as shown in formula II.

Specifically, the reaction chemical formula involved in reaction step (2) of the above trivalent iodine reagent is as follows:

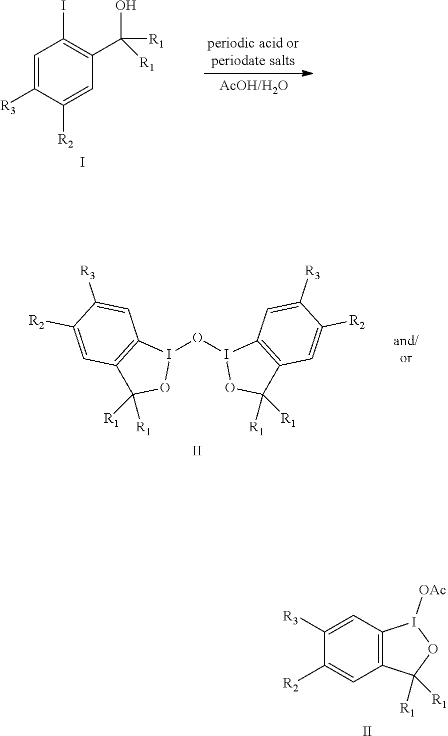

Specifically, in the above trivalent iodine reagent reaction, in step (2), R¹ is selected from methyl or trifluoromethyl, R² is selected from hydrogen, halogen, or methyl, and R³ is selected from hydrogen or methyl.

The molecular structural formula of the target product obtained from the reaction based on the above scheme is the structure of the following formula II-a and/or II-b:

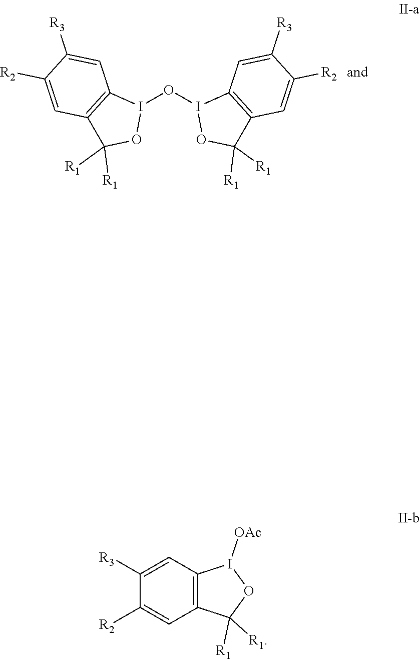

Further, for example, in the disclosure, the obtained compounds having the structure of formula II-a and/or II-b are treated with KOH and a small amount of water in DMSO, treated with TMSCl and NEt3 in DCM, or treated with TMSOTf in DCM, so as to prepare V-(a-h) shown in the glycosylation method and alkylation method of the disclosure, where the hydroxyl group, trifluoromethanesulfonyl group, and benziodoxole group are substituted on “I”.

It should be noted that the labels appearing in the preparation process of the above trivalent iodine reagent, such as R₁, R₂, R₃, II, etc., are only used to illustrate the preparation of trivalent iodine reagent. Specifically, the benziodoxole reagent prepared by the research group through the above scheme may be used in the examples of the disclosure, and the relevant details of the preparation may also refer to the public information of the research group regarding the preparation of benziodoxole reagents.

In the glycosylation method of the disclosure, the method is as follows. A glycosyl donor as shown in formula I is mixed with a benziodoxole reagent as shown in formula V in a low temperature environment, and acid is then added. After the donor is completely converted, a receptor nucleophilic reagent (IV) is added to perform a glycosylation reaction, and a glycosylation product as shown in formula VI is obtained. The reaction formula is as follows:

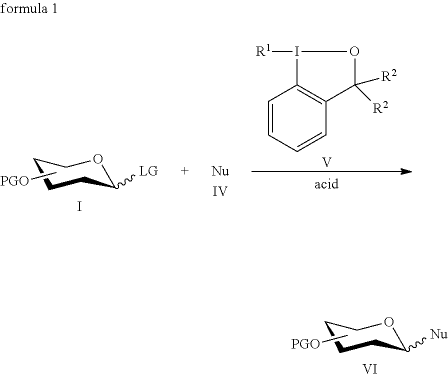

In the glycosyl donor represented by formula I, one or a plurality of hydroxyl groups on a sugar ring are protected by a glycosyl protecting group, and LG is an alkylthio group, an arylthio group, an aryl selenoyl group, a thiocarboxylate group, an o-alkynyl benzoate group, a pentenoate group, a pentynoate group, or a hexynoate group.

In the benziodoxole reagent as shown in formula V, R¹ is chlorine, hydroxyl, trimethylsilyloxy, trifluoromethanesulfonyl ester, or benziodooxolaneoxy, and R² is methyl or trifluoromethyl.

Nu in formula 1 represents the nucleophilic reagent, and the nucleophilic reagent is selected from primary alcohol, secondary alcohol, or carboxylic acid.

Preferably, in the glycosyl donor represented by formula I, the glycosyl protecting groups are each independently selected from alkyl, alkoxy, acyloxy, silyloxy, substituted amino, cyclic acetal, cyclic ketal, or glycosyl.

Preferably, LG is preferably selected from ethylthio, isopropylthio, p-methylbenzyl, benzylthio, 2-ethylphenylthio, pentynoate, hexynoate, o-alkynylbenzoate, pentenoate, or thiocarbonate.

Preferably, the benziodoxole reagent represented by formula V is selected from compounds having any one of the following structures:

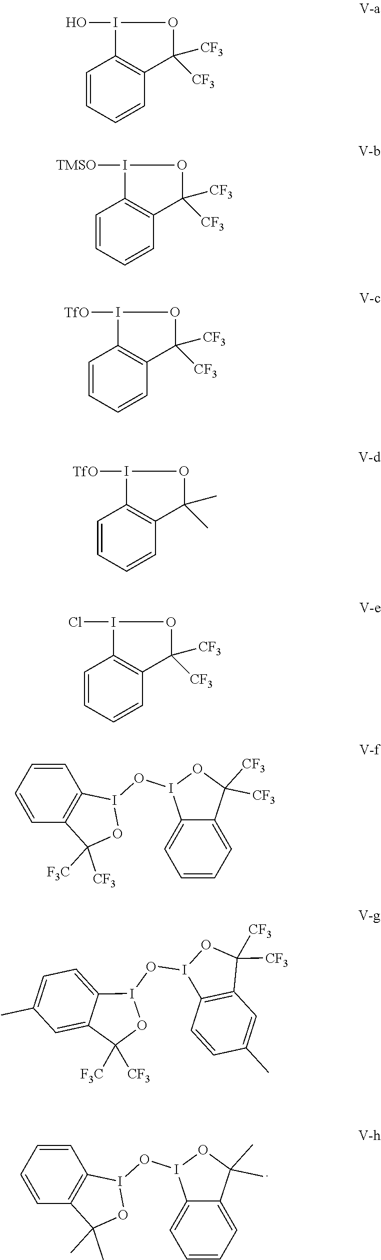

Preferably, a molar ratio of the glycosyl donor represented by formula I to the benziodoxole reagent represented by formula V is 1:(0.25 to 0.5).

Preferably, the glycosyl donor represented by Formula I is selected from compounds having any one of the following structures:

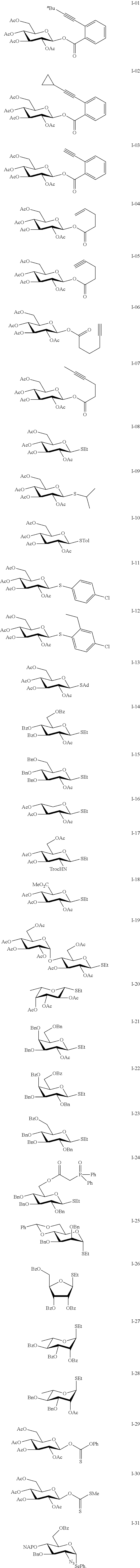

Preferably, in the glycosylation method, the nucleophilic reagent (IV) is selected from compounds having any one of the following structures:

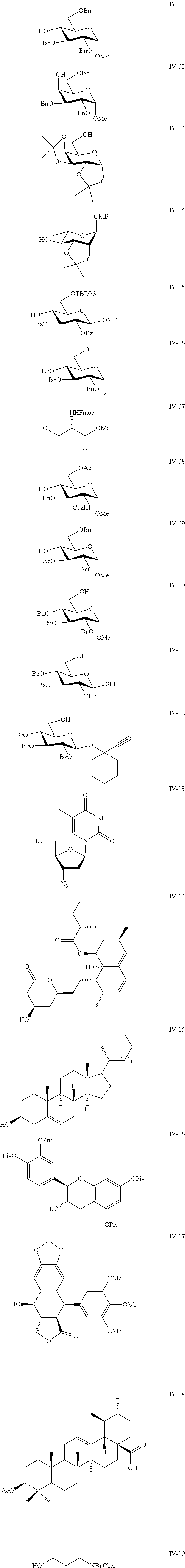

In the alkylation method of the disclosure, the method is as follows. An alkylation reagent as shown in formula II under a combined action of the benziodoxole reagent having a structure as shown in formula V and an acid catalyst are activated and then reacted with a nucleophilic reagent to obtain an alkylation product as shown in formula VII, with a reaction route as shown in formula 2. Alternatively, an alkylation reagent as shown in formula III under the combined action of the benziodoxole reagent having a structure as shown in formula V and an acid catalyst are activated and then reacted with a nucleophilic reagent to obtain an alkylation product as shown in formula VIII, with a reaction route as shown in formula 3:

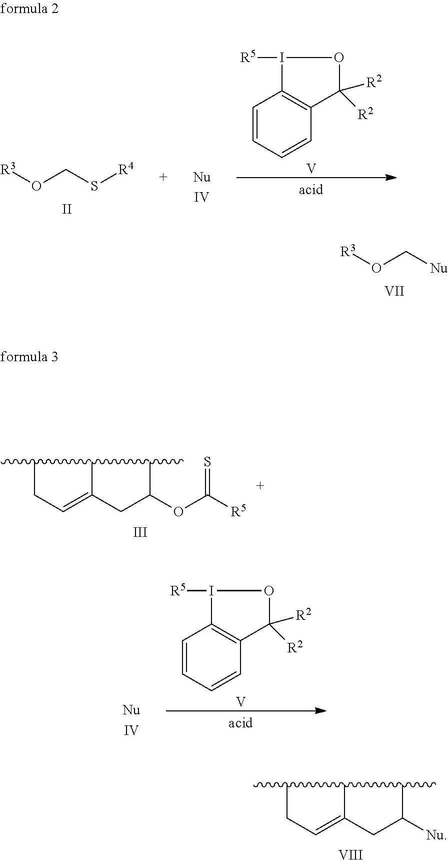

The alkylation reagent as shown in formula II is a thioether sulfide alkylation reagent, specifically when R³ is an alkyl group and R⁴ is an alkyl group. The thioether alkylating reagent is alkoxymethylene sulfide, and when R³ is an alkyl group and R⁴ is an aryl group, the thioether alkylating reagent is aryloxymethylene sulfide.

The thiocarbonate esterification reagent among the alkylation reagents represented by formula III is a steroid derivative containing a highly allyl thiocarbonate ester structure, where the steroid is cholesterol, diosgenin, dehydroepiandrosterone, pregnenolone, β-sitosterol, or stigmasterol, where R⁵ is a phenoxy group or a methylthio group.

In the benziodoxole reagent as shown in formula V, R¹ is chlorine, hydroxyl, trimethylsilyloxy, trifluoromethanesulfonyl ester, or benziodooxolaneoxy, and R² is methyl or trifluoromethyl.

Nu in both formula II and formula III represents the nucleophilic reagent, and the nucleophilic reagent is selected from primary alcohol, secondary alcohol, phenol, or phosphoric acid.

Preferably, in the alkylation method, the benziodoxole reagent represented by formula V is selected from compounds having any one of the following structures:

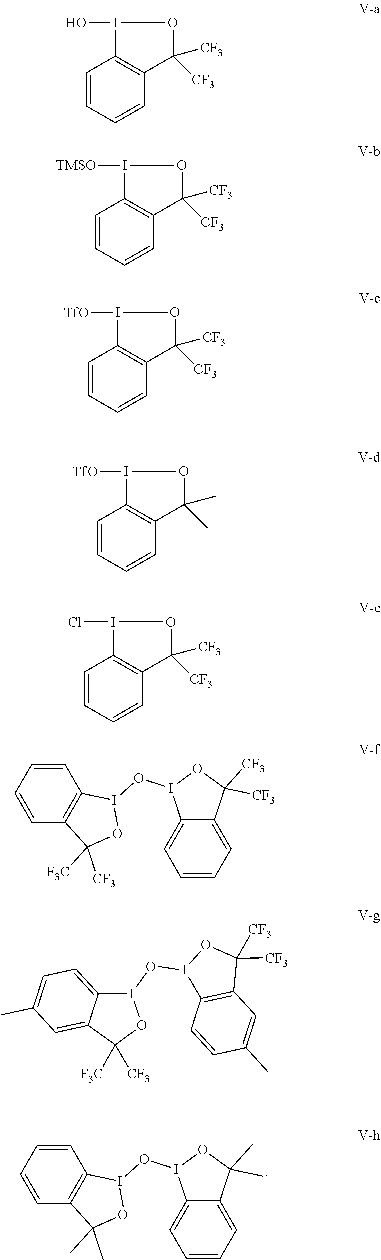

Preferably, in the above alkylation method, a molar ratio of the alkylation reagent (II or III) to the benziodoxole reagent (V) is 1:(0.25 to 0.5).

Preferably, the alkylation reagent represented by formula II is selected from compounds having any one of the following structures:

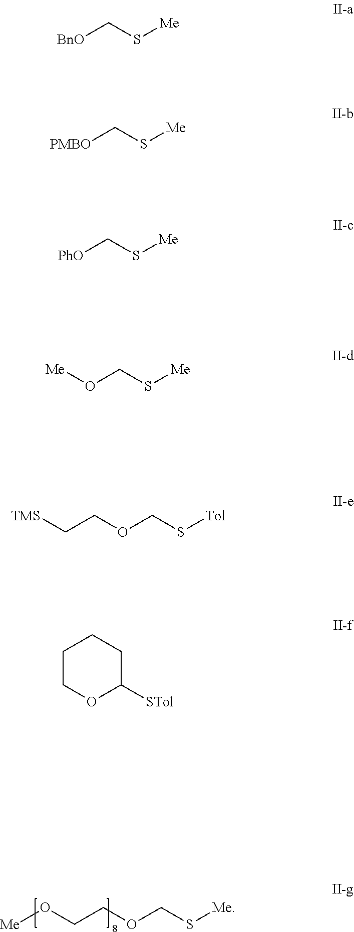

Preferably, the alkylation reagent represented by formula III is selected from compounds having any one of the following structures:

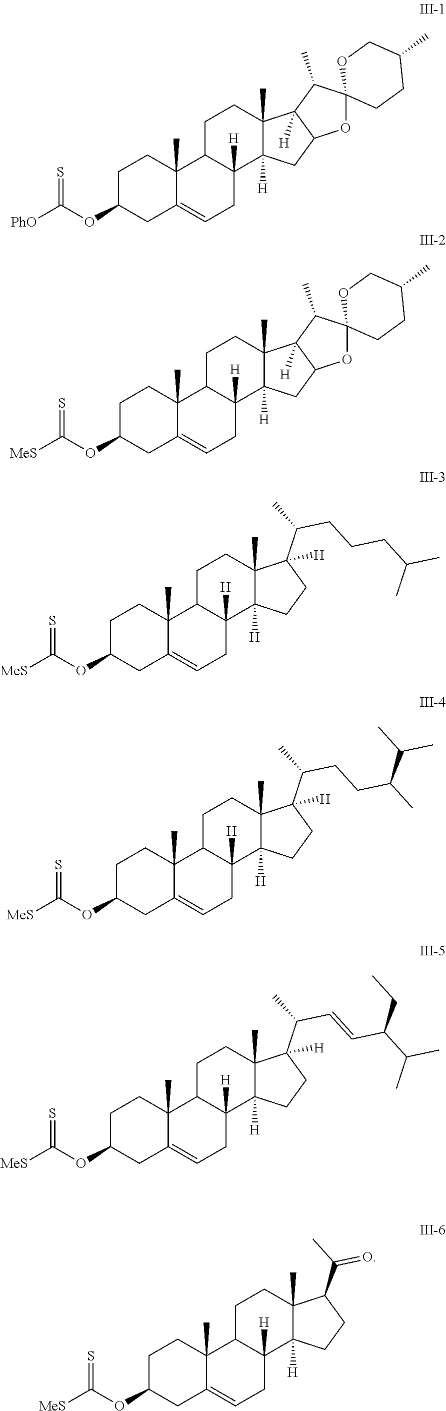

Preferably, in the alkylation method, the nucleophilic reagent (IV) is selected from primary alcohol, secondary alcohol, phenol, or phosphoric acid. The primary alcohol, secondary alcohol, or a carboxylic acid acceptor is selected from compounds having any one of the following structures:

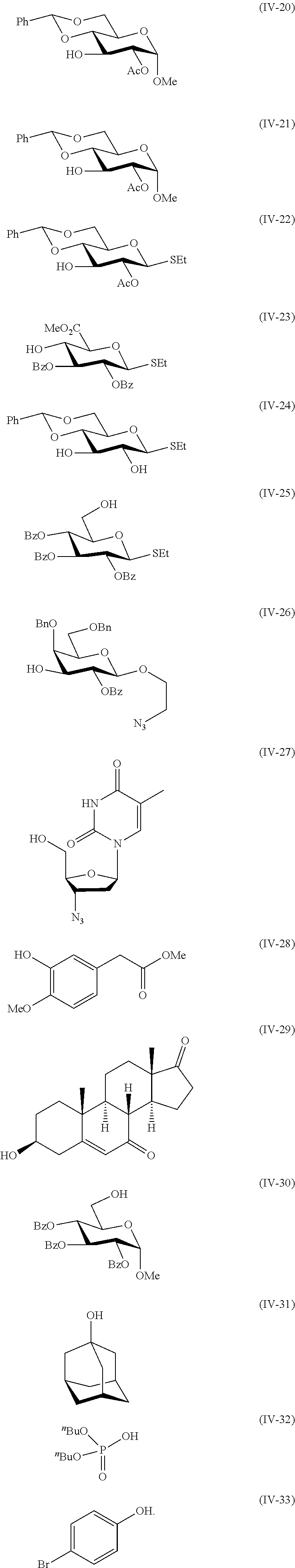

In embodiments of the disclosure, in the glycosylation method and alkylation method, the acid is selected from Lewis acid or Brønsted acid. The Lewis acid is selected from trifluoromethanesulfonic anhydride, trimethylsilyl trifluoromethanesulfonate, boron trifluoride diethyl etherate, or N-trimethylsilyl bis(trifluoromethanesulfonyl)imide, and the Brønsted acid is selected from trifluoromethanesulfonic acid or N-bis(trifluoromethanesulfonyl)imide.

In the embodiments of the disclosure, in the glycosylation method and alkylation method, the molar ratio of acid to nucleophilic reagent (IV) is 1:(0.1 to 1). Specifically, when using donors I-01, 1-02, I-03, I-04, I-05, I-06, and I-07, a stoichiometric ratio of donor to acid is 1:1. When using glycosyl donors I-08, I-09, I-10, I-11, I-12, I-13, I-14, I-15, I-16, I-17, I-18, I-19, I-20, I-21, I-22, 1-23, I-24, I-25, I-26, I-27, I-28, I-29, and I-30, the stoichiometric ratio of donor to acid is 1:(0.1 to 0.5).

In embodiments of the disclosure, in the glycosylation method and alkylation method, a heating temperature involved is −78° C. to 30° C., for example −60° C., −40° C., −20° C., −10° C., 0° C., 10° C., 25° C., or any interval range composed of these temperature values, preferably −78° C. to 25° C., for example −78° C. to 0° C., −60° C. to 0° C., −40° C. to 0° C., and −10° C. to 0° C. Specifically, a reaction temperature may be selected by a person having ordinary skill in the art based on common knowledge.

Specific examples are provided as follows:

Unless otherwise specified, the standard operating procedure for the glycosylation reaction in the examples is as follows: the glycosyl donor as described in the disclosure, benziodoxole reagent, acceptor, and dried molecular sieves 4 Å MS are placed in a reaction flask and sealed with a rubber stopper, and anhydrous CH₂Cl₂ (c=0.05 M, calculated based on the reactant with 1.0 equivalent) is then added. The reaction system is pre-cooled to 0° C., and then acid catalyst (20 mol %) is added. After the reaction is complete, a glycosylation product is obtained through separation and purification by silica gel column chromatography. In some implementation examples, where there are special instructions that are inconsistent with the standard operating procedure for the reaction, the special instructions shall prevail.

### Examples 1 to 30

In the glycosylation method of the disclosure, the detailed experimental process refers to the above description, and the following table provides the yields of different structural glycosyl donors (I) with the benziooxole reagent (V) under the action of acid and corresponding reaction conditions.

| Example | I (eq.) | V (eq.) | Acid (eq.) | Temperature | | Product | Yield |
|---|---|---|---|---|---|---|---|
| 1 | I-01(1.2) | V-c (1.5) | — | 0° | C. | VI-01 | 91% |
| 2 | I-02 (1.2) | V-c (1.5) | — | 0° | C. | VI-01 | 92% |
| 3 | I-03 (1.2) | V-c (1.5) | — | 0° | C. | VI-01 | 84% |
| 4 | I-04 (1.2) | V-c (1.5) | — | 0° | C. | VI-01 | 81% |
| 5 | I-05 (1.2) | V-c (1.5) | — | 0° | C. | VI-01 | 74% |
| 6 | I-06 (1.2) | V-c (1.5) | — | 0° | C. | VI-01 | 66% |
| 7 | I-07 (1.2) | V-c (1.5) | — | 0° | C. | VI-01 | 92% |
| 8 | I-08 (1.2) | V-c (0.6) | — | 0° | C. | VI-01 | 96% |
| 9 | I-08 (1.0) | V-f (0.25) | TMSOTf (0.5) | 0° | C. | VI-01 | 88% |
| 10 | I-08 (1.0) | V-f (0.25) | TMSOTf (0.2) | 0° | C. | VI-01 | 87% |
| 11 | I-08 (1.0) | V-f (0.25) | TMSOTf (0.1) | 0° | C. | VI-01 | 80% |
| 12 | I-08 (1.0) | V-b (0.5) | TMSOTf (0.5) | 0° | C. | VI-01 | 89% |
| 13 | I-09 (1.0) | V-f (0.25) | TMSOTf (0.2) | 0° | C. | VI-01 | 85% |
| 14 | I-10 (1.0) | V-f (0.25) | TMSOTf (0.2) | 0° | C. | VI-01 | 87% |
| 15 | I-11 (1.0) | V-f (0.25) | TMSOTf (0.2) | 0° | C. | VI-01 | 28% |
| 16 | I-12 (1.0) | V-f (0.25) | TMSOTf (0.2) | 0° | C. | VI-01 | 86% |
| 17 | I-13 (1.0) | V-f (0.25) | TMSOTf (0.2) | 0° | C. | VI-01 | 57% |
| 18 | I-07 (1.0) | V-f (0.5) | TMSOTf (1.0) | 0° | C. | VI-01 | 83% |
| 19 | I-07 (1.0) | V-a (1.0) | TMSOTf (1.0) | 0° | C. | VI-01 | 82% |
| 20 | I-07 (1.0) | V-e (1.0) | AgOTf (1.0) | 0° | C. | VI-01 | 78% |
| 21 | I-29 (1.0) | V-f (0.25) | TMSOTf (0.2) | 0° | C. | VI-01 | 72% |
| 22 | I-30 (1.0) | V-f (0.25) | TMSOTf (0.2) | 0° | C. | VI-01 | 77% |
| 23 | I-08 (1.2) | V-f (0.3) | TMSOTf (0.2) | 0° | C. | VI-01 | 96% |
| 24 | I-08 (1.2) | V-f (0.3) | TMSNTf₂ (0.2) | 0° | C. | VI-01 | 90% |
| 25 | I-08 (1.2) | V-b (0.3) | BF₃•Et₂O (0.2) | 0° | C. | VI-01 | 85% |
| 26 | I-08 (1.2) | V-h (0.3) | TMSOTf (0.6) | 25° | C. | VI-01 | 88% |
| 27 | I-29 (1.2) | V-f (0.3) | TMSOTf (0.2) | 0° | C. | VI-01 | 93% |
| 28 | I-30 (1.2) | V-f (0.3) | TMSOTf (0.2) | 0° | C. | VI-01 | 94% |
| 29 | I-08 (1.0) | V-g (0.25) | TMSOTf (0.2) | 0° | C. | VI-01 | 85% |
| 30 | I-08 (1.0) | V-d (0.5) | TMSOTf (0.2) | 25° | C. | VI-01 | 84% |

From the above examples, it can be seen that different donor structures, such as ethyl thioglycosyle, isopropyl thioglycosyle, p-methylphenyl thioglycosyle, o-ethylphenyl thioglycosyle, and pentynoic acid ester glycosyle, all exhibit good effects. Preferably, the donors are ethyl thioglycosyle, isopropyl thioglycosyle, p-methylphenyl thioglycosyle, o-ethylphenyl thioglycosyle, pentynoic acid ester glycosyle, and the like. More preferably, ethyl thioglycosyle may be used, and excellent glycosylation yields may be obtained using one-quarter equivalent of benziodoxole reagent (III-e) and a catalytic amount of acid.

Different acid catalysts may be used, such as Brønsted acids or Lewis acids, and all of which may exert catalytic effects. Preferred acid catalysts are trimethylsilyl trifluoromethanesulfonate, trifluoromethanesulfonic acid, boron trifluoride diethyl etherate, N-trimethylsilyl bis(trifluoromethanesulfonyl)imide, and the like. The amount of the acid catalyst is 5% to 30% of the glycosyl acceptor in molar percentage, more preferably 10% to 25%, for example, 10% to 20%.

### Example 31

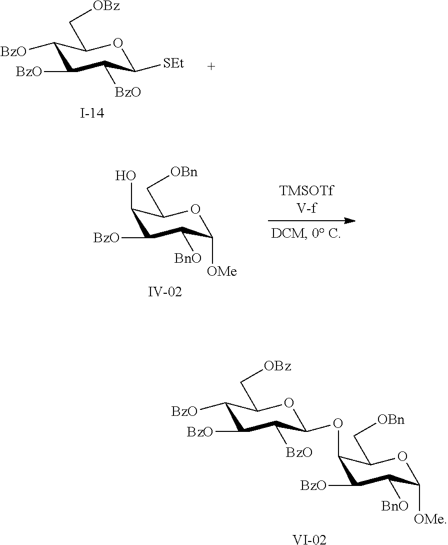

According to the reaction standard operating procedure, the raw materials are as follows: donor I-14 (33.3 mg, 0.052 mmol, 1.2 equiv), benziodoxole reagent V-f (9.7 mg, 0.013 mmol, 0.3 equiv), acceptor IV-02 (20.0 mg, 0.043 mmol, 1.0 equiv), and acid catalyst TMSOTf (20 mol %), and after the reaction is completed, the glycosylation product VI-02 (43.2 mg, 96%) is obtained. The characterization data of this compound are as follows: ¹H NMR (400 MHz, CDCl₃) δ 8.06-7.99 (m, 4H, Ar—H), 7.96-7.92 (m, 2H, Ar—H), 7.89-7.85 (m, 2H, Ar—H), 7.61-7.23 (m, 25H, Ar—H), 7.02-7.00 (m, 2H, Ar—H), 5.90 (t, J=9.6 Hz, 1H), 5.72 (t, J=9.6 Hz, 1H), 5.60 (dd, J=9.6, 8.0 Hz, 1H, H−2), 5.27 (d, J=8.0 Hz, 1H, H−1), 4.75 (d, J=12.0 Hz, 1H, —CH₂Ph), 4.66 (d, J=12.0 Hz, 1H, —CH₂Ph), 4.61 (dd, J=12.0, 3.2 Hz, 1H), 4.50 (d, J=3.6 Hz, 1H, H−1′), 4.47 (s, 2H), 4.44 (dd, J=12.0, 4.4 Hz, 1H), 4.13 (d, J=11.6 Hz, 2H, —CH₂Ph), 4.03 (dt, J=10.0, 3.6 Hz, 1H), 3.93-3.87 (m, 2H), 3.84 (dd, J=10.0, 2.8 Hz, 1H), 3.73 (dd, J=10.0, 4.8 Hz, 1H), 3.64 (dd, J=10.0, 6.8 Hz, 1H), 3.59 (dd, J=10.0, 3.6 Hz, 1H), 3.32 (s, 3H, —OCH₃).

### Example 32

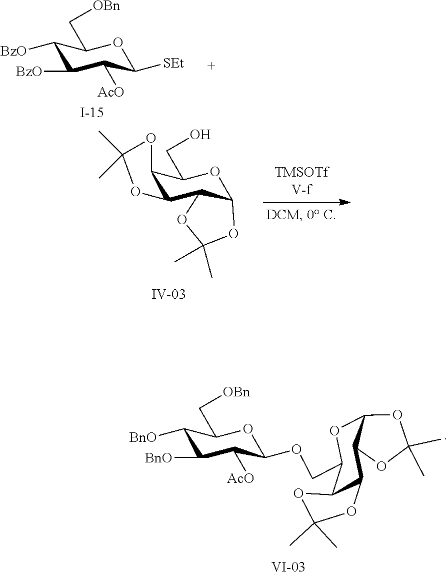

According to the reaction standard operating procedure, the raw materials were as follows: donor I-15 (27.9 mg, 0.052 mmol, 1.2 equiv), benziodoxole reagent V-f (9.7 mg, 0.013 mmol, 0.3 equiv), acceptor IV-03 ((11.2 mg, 0.043 mmol, 1.0 equiv), and acid catalyst TMSOTf (20 mol %), and after the reaction was completed, the glycosylation product VI-03 (30.3 mg, 96%) was obtained. The characterization data of this compound are as follows: ¹H NMR (400 MHz, CDCl₃) δ 7.31-7.21 (m, 13H, Ar—H), 7.16-7.12 (m, 2H, Ar—H), 5.46 (d, J=4.8 Hz, 1H, H−1′), 4.96 (t, J=8.4 Hz, 1H, H−2), 4.75 (d, J=10.8 Hz, 1H, —CH₂Ph), 4.74 (d, J=11.6 Hz, 1H, —CH₂Ph), 4.64 (d, J=11.2 Hz, 1H, —CH₂Ph), 4.60 (d, J=12.4 Hz, 1H, —CH₂Ph), 4.54-4.49 (m, 3H), 4.40 (d, J=8.0 Hz, 1H, H−1), 4.24 (dd, J=4.8, 2.4 Hz, 1H, H−2′), 4.14 (dd, J=8.0, 1.6 Hz, 1H), 4.02 (dd, J=11.2, 3.2 Hz, 1H), 3.91-3.86 (m, 1H), 3.70-3.56 (m, 5H), 3.45-3.41 (m, 1H), 1.97 (s, 3H, —COCH₃), 1.46 (s, 3H, —CH₃), 1.39 (s, 3H, —CH₃), 1.27 (s, 3H, —CH₃), 1.26 (s, 3H, —CH₃).

### Example 33

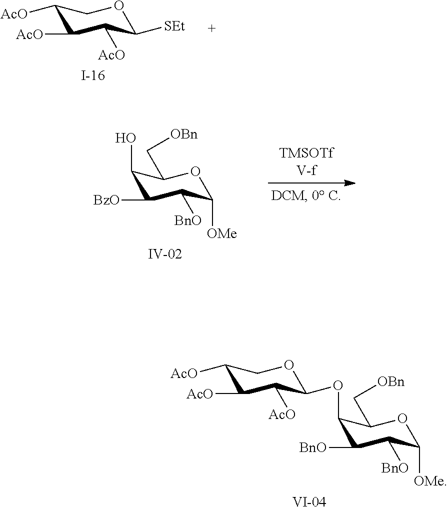

According to the reaction standard operating procedure, the raw materials were as follows: donor I-16 (16.7 mg, 0.052 mmol, 1.2 equiv), benziodoxole reagent V-f (9.7 mg, 0.013 mmol, 0.3 equiv), acceptor IV-02 (20.0 mg, 0.043 mmol, 1.0 equiv), and acid catalyst TMSOTf (20 mol %), and after the reaction was completed, the glycosylation product VI-04 (29.5 mg, 95%) was obtained. The characterization data of this compound are as follows: ¹H NMR (600 MHz, CDCl₃) δ 7.39-7.26 (m, 15H, Ar—H), 5.19 (t, J=9.0 Hz, 1H, H−3′), 4.98 (dd, J=9.0, 7.2 Hz, 1H, H−2′), 4.92 (td, J=8.4, 5.4 Hz, 1H, H−4′), 4.80 (d, J=12.0 Hz, 1H, —CH₂Ph), 4.79 (d, J=7.2 Hz, 1H, H−1′), 4.76 (d, J=12.0 Hz, 1H, —CH₂Ph), 4.67 (d, J=11.4 Hz, 1H, —CH₂Ph), 4.60 (d, J=3.6 Hz, 1H, H−1), 4.58 (d, J=12.0 Hz, 1H, —CH₂Ph), 4.53 (d, J=11.4 Hz, 1H, —CH₂Ph), 4.49 (d, J=11.4 Hz, 1H, —CH₂Ph), 4.06 (d, J=1.8 Hz, 1H, H−4), 4.02 (dd, J=12.0, 5.4 Hz, 1H, H−5′a), 3.89-3.84 (m, 2H, H−3, H−5), 3.78 (dd, J=10.2, 3.6 Hz, 1H, H−2), 3.69 (dd, J=9.6, 6.6 Hz, 1H, H−6a), 3.55 (dd, J=9.6, 6.0 Hz, 1H, H−6b), 3.35 (s, 3H, —OCH₃), 3.24 (dd, J=12.0, 8.4 Hz, 1H, H−5b), 2.05 (s, 3H, —COCH₃), 2.01 (s, 3H, —COCH₃), 1.80 (s, 3H, —COCH₃). ¹³C NMR (100 MHz, CDCl₃) δ 170.2, 170.1, 169.7, 138.6, 138.6, 138.4, 128.6, 128.5, 128.5, 128.1, 127.9, 127.8, 127.8, 127.8, 101.9, 98.7, 78.3, 75.8, 73.8, 73.7, 73.6, 71.6, 71.2, 696, 69.4, 68.9, 62.2, 55.5, 20.9, 20.9, 20.8. HRMS (ESI⁺): calc, for C₃₉H₄₆O₁₃ [M+Na]⁺745.2831, found: 745.2824.

### Example 34

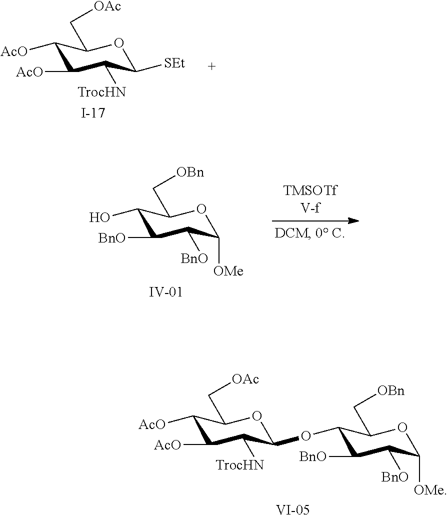

According to the reaction standard operating procedure, the raw materials were as follows: donor I-17 (27.3 mg, 0.052 mmol, 1.2 equiv), benziodoxole reagent V-f (9.7 mg, 0.013 mmol, 0.3 equiv), acceptor IV-01 (20.0 mg, 0.043 mmol, 1.0 equiv), and acid catalyst TMSOTf (20 mol %), and after reaction was completed, the glycosylation product VI-05 (39.1 mg, 98%) was obtained. The characterization data of this compound are as follows: ¹H NMR (600 MHz, CDCl₃) δ 7.52-7.20 (m, 15H), 4.97 (d, J=11.4 Hz, 1H), 4.90 (t, J=9.6 Hz, 1H), 4.83 (d, J=12.0 Hz, 1H), 4.77-4.67 (m, 4H), 4.60-4.53 (m, 3H), 4.32 (d, J=12.0 Hz, 1H), 4.20 (d, J=8.4 Hz, 1H), 4.10 (dd, J=12.0, 4.2 Hz, 1H), 3.93 (d, J=9.6 Hz, 1H), 3.83-3.77 (m, 3H), 3.66 (dd, J=10.8, 2.4 Hz, 1H), 3.60-3.57 (m, 1H), 3.53 (q, J=9.6 Hz, 1H), 3.47-3.42 (m, 2H), 3.40-3.36 (m, 1H), 3.32 (s, 3H), 1.99 (s, 3H), 1.96 (s, 3H), 1.90 (s, 3H).

### Example 35

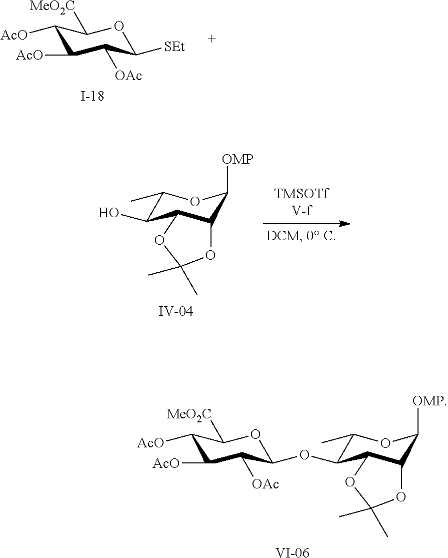

According to the reaction standard operating procedure, the raw materials were as follows: donor I-18 (19.7 mg, 0.052 mmol, 1.2 equiv), benziodoxole reagent V-f (9.7 mg, 0.013 mmol, 0.3 equiv), acceptor IV-04 (13.9 mg, 0.043 mmol, 1.0 equiv), and acid catalyst TMSOTf (20 mol %), and after the reaction was completed, the glycosylation product VI-06 (25.5 mg, 93%) was obtained. The characterization data of this compound are as follows: ¹H NMR (600 MHz, CDCl₃) δ 6.97-6.93 (m, 2H, Ar—H), 6.82-6.79 (m, 2H, Ar—H), 5.54 (s, 1H, H−1), 5.27 (t, J=9.2 Hz, 1H, H−3′), 5.21 (t, J=9.6 Hz, 1H, H−4′), 5.03 (d, J=7.8 Hz, 1H, H−1′), 4.96 (dd, J=9.0, 8.4 Hz, 1H, H−2′), 4.28 (d, J=5.6 Hz, 1H, H−2), 4.20 (dd, J=7.2, 5.6 Hz, 1H, H−3), 3.99 (d, J=9.6 Hz, 1H, H−5′), 3.79-3.74 (m, 4H), 3.72 (s, 3H), 3.61 (dd, J=10.0, 7.6 Hz, 1H, H−4), 2.06 (s, 3H, —COCH₃), 2.00 (s, 3H, —COCH₃), 1.99 (s, 3H, —COCH₃), 1.51 (s, 3H, —CH₃), 1.36 (s, 3H, —CH₃), 1.19 (d, J=6.0 Hz, 3H, —CH₃-6). ¹³C NMR (150 MHz, CDCl₃) δ 170.3, 169.6, 169.5, 167.3, 155.2, 150.2, 118.0, 114.8, 109.8, 99.6, 96.3, 79.7, 78.0, 76.2, 72.6, 72.4, 71.5, 69.7, 64.9, 55.8, 52.9, 28.0, 26.6, 20.9, 20.8, 20.7, 17.5. HRMS (ESI⁺): calc, for C₂₉H₃₈O₁₅ [M+Na]⁺649.2103, found: 649.2103.

### Example 36

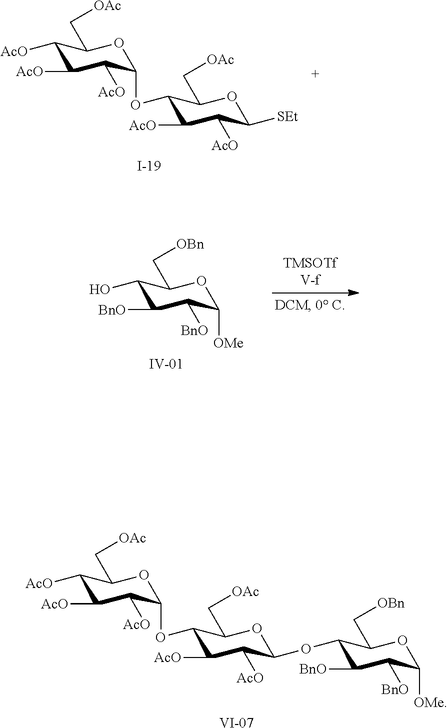

According to the reaction standard operating procedure, the raw materials were as follows: donor I-19 (35.4 mg, 0.052 mmol, 1.2 equiv), benziodoxole reagent V-f (9.7 mg, 0.013 mmol, 0.3 equiv), acceptor IV-01 (20.0 mg, 0.043 mmol, 1.0 equiv), and acid catalyst TMSOTf (20 mol %), and after the reaction was completed, the glycosylation product VI-07 (45.2 mg, 97%) was obtained. The characterization data of this compound are as follows: ¹H NMR (600 MHz, CDCl₃) δ 7.45-7.36 (m, 5H, Ar—H), 7.33-7.21 (m, 10H, Ar—H), 5.37 (t, J=10.2 Hz, 1H), 5.31 (d, J=3.6 Hz, 1H), 5.04 (t, J=10.2 Hz, 1H), 5.00 (t, J=9.0 Hz, 1H), 4.94 (d, J=11.4 Hz, 1H), 4.85 (dd, J=10.8, 3.6 Hz, 1H), 4.76 (d, J=12.0 Hz, 1H), 4.73-4.66 (m, 3H), 4.58-4.53 (m, 2H), 4.46 (d, J=7.8 Hz, 1H, H−1′), 4.40 (d, J=12.0 Hz, 1H), 4.20 (dd, J=12.6, 3.6 Hz, 1H), 4.12 (dd, J=12.0, 2.4 Hz, 1H), 4.05 (dd, J=12.0, 3.6 Hz, 1H), 3.97 (dd, J=12.6, 1.8 Hz, 1H), 3.88-3.82 (m, 3H), 3.81-3.75 (m, 2H), 3.62-3.57 (m, 2H), 3.45 (dd, J=9.6, 3.6 Hz, 1H), 3.35 (s, 3H), 3.14 (dt, J=9.6, 3.0 Hz, 1H), 2.09 (s, 3H), 2.06 (s, 3H), 2.02 (s, 3H), 2.00 (s, 3H), 1.96 (s, 3H), 1.93 (s, 6H).

### Example 37

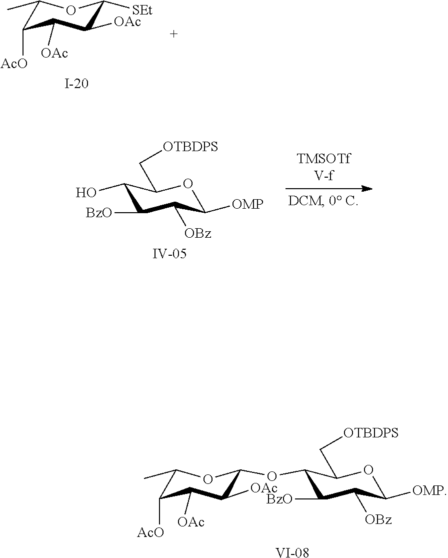

According to the reaction standard operating procedure, the raw materials were as follows: donor I-20 (17.4 mg, 0.052 mmol, 1.2 equiv), benziodoxole reagent V-f (9.7 mg, 0.013 mmol, 0.3 equiv), acceptor IV-05 (31.5 mg, 0.043 mmol, 1.0 equiv), and acid catalyst TMSOTf (20 mol %), and after the reaction was completed, the glycosylation product VI-08 (40.6 mg, 94%) was obtained. The characterization data of this compound are as follows: ¹H NMR (400 MHz, CDCl₃) δ 7.94-7.87 (m, 4H, Ar—H), 7.73-7.67 (m, 4H, Ar—H), 7.55-7.46 (m, 2H, Ar—H), 7.41-7.29 (m, 10H, Ar—H), 7.02-6.94 (m, 2H, Ar—H), 6.70-6.61 (m, 2H, Ar—H), 5.71 (t, J=9.6 Hz, 1H), 5.51 (dd, J=9.9, 8.0 Hz, 1H), 5.14 (d, J=8.0 Hz, 1H, H−1′), 5.02 (d, J=3.2 Hz, 1H), 4.98 (dd, J=10.4, 8.0 Hz, 1H), 4.56 (dd, J=10.4, 3.2 Hz, 1H), 4.44 (d, J=8.0 Hz, 1H, H−1), 4.34 (d, J=9.6 Hz, 1H), 3.94 (t, J=9.2 Hz, 1H), 3.88-3.79 (m, 2H), 3.68 (s, 3H, —OCH₃), 3.55 (m, 1H, H−5′), 2.00 (s, 3H, —COCH₃), 1.89 (s, 3H, —COCH₃), 1.86 (s, 3H, —COCH₃), 1.09 (s, 9H, —C(CH₃) 3), 0.85 (d, J=6.4 Hz, 3H, —CH₃). ¹³C NMR (101 MHz, CDCl₃) δ 170.6, 170.2, 169.9, 165.6, 165.5, 155.5, 151.6, 135.8, 135.8, 133.9, 133.6, 133.6, 133.3, 129.9, 129.7, 129.7, 129.6, 129.4, 129.2, 128.8, 128.5, 127.8, 127.7, 118.5, 114.6, 100.9, 100.3, 76.3, 75.1, 74.3, 72.3, 71.4, 70.0, 69.1, 68.5, 63.6, 55.7, 27.1, 20.7, 20.7, 20.6, 19.4, 15.8. HRMS (ESI⁺): calc, for C₅₅H₆₀O₁₆Si [M+Na]⁺1027.3543, found: 1027.3530.

### Example 38

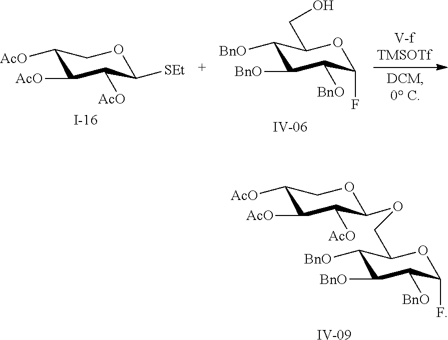

According to the reaction standard operating procedure, the raw materials were as follows: donor I-16 (16.7 mg, 0.052 mmol, 1.2 equiv), benziodoxole reagent V-f (9.7 mg, 0.013 mmol, 0.3 equiv), acceptor IV-06 (19.5 mg, 0.043 mmol, 1.0 equiv), and acid catalyst TMSOTf (20 mol %), and after the reaction was completed, the glycosylation product VI-09 (27.8 mg, 91%) was obtained. The characterization data of this compound are as follows: ¹H NMR (400 MHz, CDCl₃) δ 7.33-7.22 (15H, m, Ar—H), 5.50 (1H, dd, J=2.8, 53.2 Hz, H−1), 5.13 (1H, t, J=8.8, 8.4 Hz), 4.94 (3H, m), 4.80 (3H, m, PhCH₂), 4.67 (1H, d, J=12.0 Hz, PhCH₂), 4.53 (1H, d, J=10.8 Hz, PhCH₂), 4.48 (1H, d, J=6.8 Hz), 4.09 (1H, dd, J=4.8, 11.6 Hz, H−6a), 4.00 (1H, dd, J=1.6, 11.2 Hz, H−6b), 3.95 (1H, t, J=9.2, 9.6 Hz), 3.90 (1H, m, H−5), 3.76 (1H, dd, J=3.6, 11.2 Hz, H−5a′), 3.57 (1H, t, J=9.6, 10.0 Hz), 3.50 (1H, ddd, J=2.4, 9.6, 12.0 Hz, H−2), 3.29 (1H, dd, J=8.8, 11.6 Hz, H−5b′), 2.02 (3H, s, CH₃), 2.02 (3H, s, CH₃), 1.99 (3H, s, CH₃), 1.92 (3H, s, CH₃). ¹³C NMR (100 MHz, CDCl₃) δ 170.33, 170.00, 169.46, 138.58, 138.07, 137.76, 128.78, 128.64, 128.32, 128.26, 128.19, 128.10, 127.94, 106.77, 100.61, 81.48, 79.51 (d, J=20 Hz), 76.69, 76.00, 75.34, 73.79, 72.44, 72.40, 71.77, 70.96, 68.91, 67.19, 20.94, 20.91, 20.84. HRMS (ESI⁺): calc. for C₃₈H₄₃FNaO₁₂ [M+Na]⁺733.2631, found: 733.2611.

### Example 39

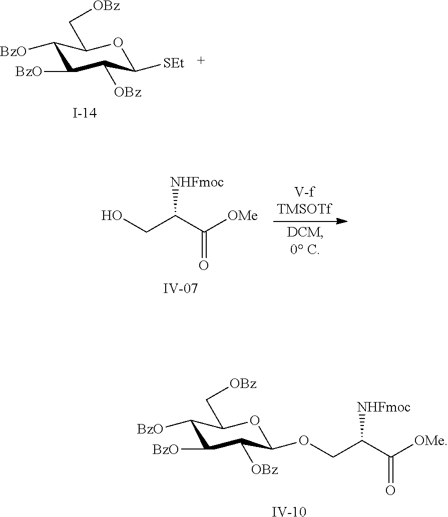

According to the reaction standard operating procedure, the raw materials were as follows: donor I-14 (33.3 mg, 0.052 mmol, 1.2 equiv), benziodoxole reagent V-f (9.7 mg, 0.013 mmol, 0.3 equiv), acceptor IV-07 (14.7 mg, 0.043 mmol, 1.0 equiv), and acid catalyst TMSOTf (20 mol %), and after the reaction was completed, the glycosylation product VI-10 (36.9 mg, 93%) was obtained. The characterization data of this compound are as follows: ¹H NMR (400 MHz, CDCl₃) δ 8.05-7.24 (m, 28H, Ar—H), 5.90 (t, J=9.6 Hz, 1H), 5.65 (t, J=9.6 Hz, 1H), 5.56-5.43 (m, 2H), 4.77 (d, J=7.6 Hz, 1H, H−1), 4.63 (dd, J=12.0, 3.2 Hz, 1H), 4.50-4.41 (m, 2H), 4.37-4.22 (m, 3H), 4.17-4.07 (m, 2H), 3.88 (dd, J=10.4, 3.2 Hz, 1H), 3.65 (s, 3H, —OCH₃).

### Example 40

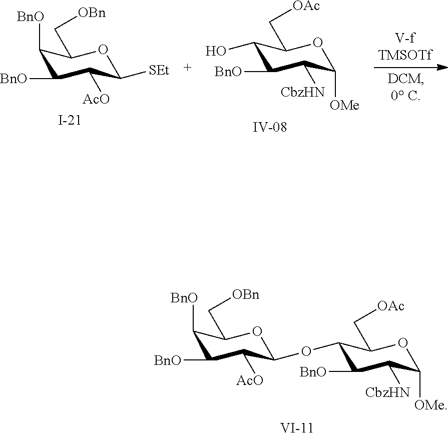

According to the reaction standard operating procedure, the raw materials were as follows: donor I-21 (27.9 mg, 0.052 mmol, 1.2 equiv), benziodoxole reagent V-f (9.7 mg, 0.013 mmol, 0.3 equiv), acceptor IV-08 (19.8 mg, 0.043 mmol, 1.0 equiv), and acid catalyst TMSOTf (20 mol %), and after the reaction was completed, the glycosylation product VI-11 (38.2 mg, 95%) was obtained. The characterization data of this compound are as follows: ¹H NMR (400 MHz, CDCl₃) δ 7.31-7.06 (m, 25H, Ar—H), 5.32 (dd, J=10.0, 8.0 Hz, 1H, H−2′), 5.03 (d, J=12.0 Hz, 1H, —CH₂Ph), 4.99 (d, J=12.4 Hz, 1H, —CH₂Ph), 4.91 (d, J=11.6 Hz, 1H, —CH₂Ph), 4.88 (d, J=11.6 Hz, 1H, —CH₂Ph), 4.77 (d, J=10.0 Hz, 1H), 4.61 (d, J=12.0 Hz, 1H, —CH₂Ph), 4.59 (d, J=3.6 Hz, 1H, H−1), 4.49 (d, J=11.6 Hz, 1H, —CH₂Ph), 4.45 (d, J=11.2 Hz, 1H, —CH₂Ph), 4.43 (d, J=12.0 Hz, 1H, —CH₂Ph), 4.37-4.32 (m, 2H, H−1′, —CH₂Ph), 4.20 (d, J=11.6 Hz, 1H, —CH₂Ph), 4.13 (d, J=12.0 Hz, 1H, —CH₂Ph), 4.06 (dd, J=11.6, 4.0 Hz, 1H), 3.90-3.84 (m, 2H), 3.70-3.63 (m, 2H), 3.48 (dd, J=10.4, 8.4 Hz, 1H), 3.40 (dd, J=10.0, 2.8 Hz, 1H), 3.38-3.30 (m, 2H), 3.26 (s, 3H, —OCH₃), 3.22-3.18 (m, 1H), 2.02 (s, 3H, —COCH₃), 1.99 (s, 3H, —COCH₃). ¹³C NMR (101 MHz, CDCl₃) δ 170.8, 169.8, 156.1, 139.2, 138.8, 138.0, 136.4, 128.6, 128.6, 128.5, 128.3, 128.1, 128.0, 128.0, 127.9, 127.9, 127.7, 127.6, 127.4, 127.1, 101.6, 98.8, 80.6, 78.7, 78.1, 75.1, 74.7, 73.6, 73.5, 72.7, 72.1, 72.0, 69.0, 68.1, 67.0, 62.6, 55.3, 54.6, 21.1, 21.0. HRMS (ESI⁺): calc, for C₅₃H₅₉NO₁₄ [M+Na]⁺956.3828, found: 956.3828.

### Example 41

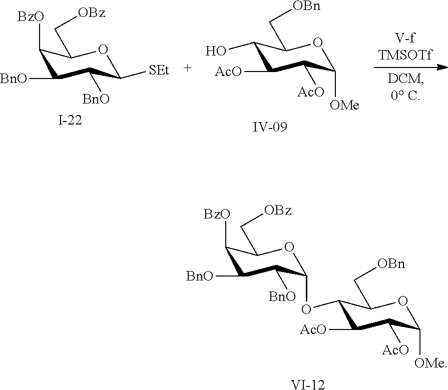

According to the reaction standard operating procedure, the raw materials were as follows: donor I-22 (31.9 mg, 0.052 mmol, 1.2 equiv), benziodoxole reagent V-f (9.7 mg, 0.013 mmol, 0.3 equiv), acceptor IV-09 (15.8 mg, 0.043 mmol, 1.0 equiv), and acid catalyst TMSOTf (20 mol %), and after the reaction was completed, the glycosylation product VI-12 (38.3 mg, 96%) was obtained. The characterization data of this compound are as follows: ¹H NMR (400 MHz, CDCl₃) δ 8.03-7.96 (m, 4H, Ar—H), 7.60-7.37 (m, 7H, Ar—H), 7.32-7.24 (m, 14H, Ar—H), 5.72 (d, J=3.2 Hz, 1H, H−1′), 5.52 (dd, J=10.0, 9.2 Hz, 1H, H−3), 4.97 (d, J=3.2 Hz, 1H, H−4′), 4.91 (d, J=3.6 Hz, 1H, H−1), 4.86 (dd, J=10.0, 3.6 Hz, 1H, H−2), 4.76 (d, J=11.2 Hz, 1H, —CH₂Ph), 4.65 (d, J=12.0 Hz, 1H, —CH₂Ph), 4.58 (d, J=12.0 Hz, 1H, —CH₂Ph), 4.53 (d, J=12.0 Hz, 1H, —CH₂Ph), 4.52 (d, J=11.2 Hz, 1H, —CH₂Ph), 4.44 (d, J=12.0 Hz, 1H, —CH₂Ph), 4.40-4.33 (m, 2H), 4.18 (dd, J=9.6, 3.2 Hz, 1H), 4.03-3.97 (m, 2H, H−4, H−5), 3.93 (dd, J=10.0, 3.2 Hz, 1H, H−2′), 3.75 (dd, J=10.0, 3.2 Hz, 1H, H−3′), 3.69 (d, J=9.6 Hz, 1H), 3.44 (dd, J=11.2, 1.6 Hz, 1H), 3.32 (s, 3H, —OCH₃), 2.05 (s, 3H, —COCH₃), 1.81 (s, 3H, —COCH₃). ¹³C NMR (100 MHz, CDCl₃) δ 170.5, 170.5, 166.0, 165.8, 138.3, 138.3, 137.9, 133.3, 130.1, 130.0, 129.8, 129.8, 128.5, 128.5, 128.5, 128.4, 128.4, 128.2, 128.0, 128.0, 127.8, 127.6, 98.8, 96.8, 76.4, 75.8, 73.7, 73.5, 72.6, 71.2, 70.7, 70.2, 69.0, 68.4, 68.1, 63.3, 55.3, 21.1, 20.9. HRMS (ESI⁺): calc, for C₅₂H₅₄O₁₅ [M+Na]⁺941.3355, found: 941.3351.

### Example 42

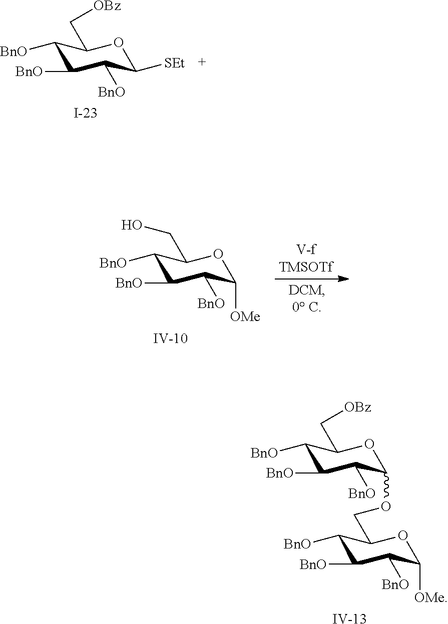

According to the reaction standard operating procedure, the raw materials were as follows: donor I-23 (31.1 mg, 0.052 mmol, 1.2 equiv), benziodoxole reagent V-f (9.7 mg, 0.013 mmol, 0.3 equiv), acceptor IV-10 (20.0 mg, 0.043 mmol, 1.0 equiv), and acid catalyst TMSOTf (20 mol %), and after the reaction was completed, the glycosylation product VI-13 (42.2 mg, 96%) was obtained. The characterization data of this compound are as follows: ¹H NMR (600 MHz, CDCl₃) δ 7.97 (d, J=7.2 Hz, 2H), 7.53 (t, J=7.2 Hz, 1H), 7.39 (t, J=7.8 Hz, 2H), 7.36-7.24 (m, 26H), 7.24-7.18 (m, 4H), 4.98-4.94 (m, 3H), 4.93-4.89 (m, 2H), 4.82-4.78 (m, 2H), 4.71-4.66 (m, 3H), 4.63-4.54 (m, 4H), 4.49 (d, J=12.0 Hz, 1H), 4.40-4.36 (m, 1H), 4.04-3.96 (m, 3H), 3.82-3.77 (m, 2H), 3.70 (d, J=10.4 Hz, 1H), 3.61-3.53 (m, 3H), 3.43-3.38 (m, 1H), 3.34 (s, 3H).

### Example 43

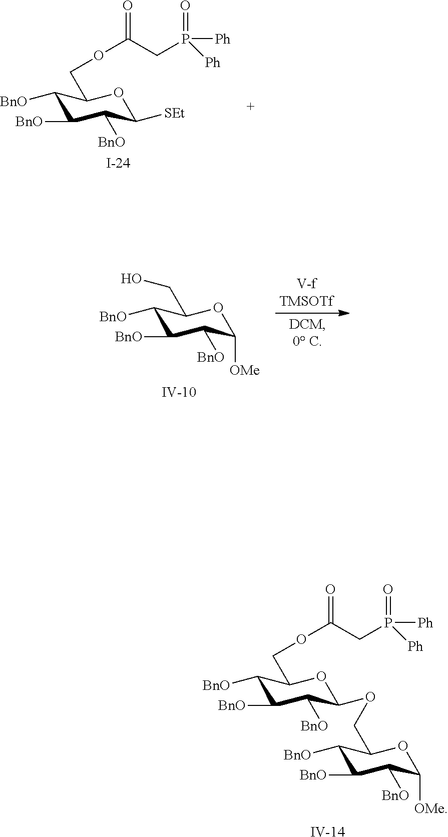

According to the reaction standard operating procedure, the raw materials were as follows: donor I-24 (38.3 mg, 0.052 mmol, 1.2 equiv), benziodoxole reagent V-f (9.7 mg, 0.013 mmol, 0.3 equiv), acceptor IV-10 (20.0 mg, 0.043 mmol, 1.0 equiv), and acid catalyst TMSOTf (20 mol %), and after the reaction was completed, the glycosylation product VI-14 (48.0 mg, 98%) was obtained. The characterization data of this compound are as follows: ¹H NMR (400 MHz, CDCl₃) δ 7.76-7.69 (m, 4H), 7.47-7.38 (m, 6H), 7.35-7.11 (m, 30H), 4.94 (d, J=10.8 Hz, 2H), 4.89 (d, J=10.8 Hz, 1H), 4.78-4.67 (m, 6H), 4.64 (d, J=12.0 Hz, 1H), 4.58 (d, J=3.6 Hz, 1H), 4.47 (d, J=11.2 Hz, 1H), 4.40 (d, J=10.8 Hz, 1H), 4.28-4.21 (m, 2H), 4.13-4.05 (m, 2H), 3.96 (t, J=9.2 Hz, 1H), 3.81-3.76 (m, 1H), 3.60 (dd, J=10.8, 4.8 Hz, 1H), 3.57-3.52 (m, 1H), 3.52-3.39 (m, 4H), 3.39-3.32 (m, H), 3.30 (s, 3H), 3.30-3.25 (m, 1H).

### Example 44

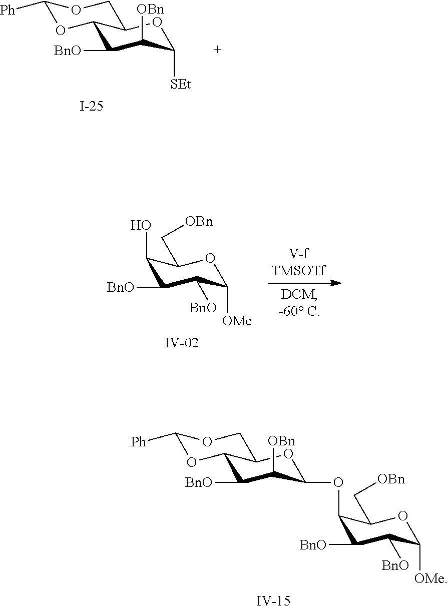

According to the reaction standard operating procedure, the raw materials were as follows: donor I-25 (25.5 mg, 0.052 mmol, 1.2 equiv), benziodoxole reagent V-f (9.7 mg, 0.013 mmol, 0.3 equiv), acceptor IV-02 (20.0 mg, 0.043 mmol, 1.0 equiv), and acid catalyst TMSOTf (20 mol %), and after the reaction was completed, the glycosylation product VI-15 (37.3 mg, 97%) was obtained. The characterization data of this compound are as follows: ¹H NMR (400 MHz, CDCl₃) δ 7.43-7.16 (m, 30H, Ar—H), 5.50 (s, 1H), 4.78 (d, J=12.4 Hz, 1H, —CH₂Ph), 4.78 (d, J=11.6 Hz, 1H, —CH₂Ph), 4.70 (d, J=3.6 Hz, 1H, H−1), 4.69-4.63 (m, 3H, H−1′, 2×-CH₂Ph), 4.59-4.51 (m, 3H, 3×-CH₂Ph), 4.49 (d, J=12.0 Hz, 1H, —CH₂Ph), 4.47-4.41 (m, 2H, 2×-CH₂Ph), 4.12-4.02 (m, 3H), 3.91-3.82 (m, 3H), 3.79-3.73 (m, 2H), 3.69 (dd, J=10.0, 5.6 Hz, 1H), 3.61 (dd, J=10.0, 6.4 Hz, 1H), 3.35 (s, 3H), 3.32 (dd, J=10.0, 3.0 Hz, 1H), 3.09 (td, J=10.0, 4.8 Hz, 1H). ¹³C NMR (100 MHz, CDCl₃) δ 139.0, 138.6, 138.6, 138.5, 138.4, 137.7, 129.0, 128.7, 128.5, 128.5, 128.4, 128.4, 128.3, 128.2, 128.1, 128.0, 127.9, 127.7, 127.7, 127.6, 127.5, 127.5, 126.2, 102.8, 101.4, 98.5, 78.6, 78.6, 78.4, 76.5, 75.4, 75.0, 73.8, 73.4, 73.4, 72.3, 69.8, 69.3, 68.7, 67.7, 55.5. HRMS (ESI⁺): calc, for C₅₅H₅₈O₁₁ [M+Na]⁺917.3871, found: 917.3884.

### Example 45

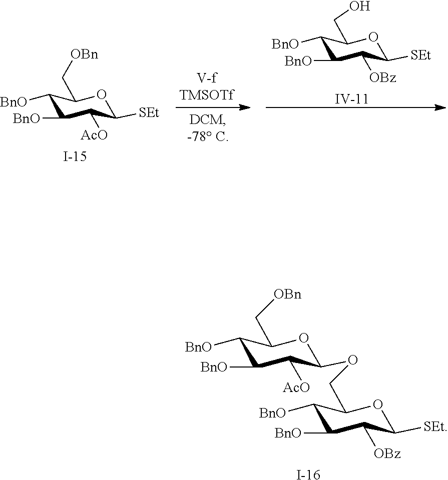

According to the reaction standard operating procedure, donor I-15 (23.1 mg, 0.043 mmol, 1.0 equivalent), benziodoxole reagent V-f (8.1 mg, 0.011 mmol, 0.25 equivalent), and molecular sieves were added to a reaction flask and sealed with a rubber stopper, and anhydrous CH₂Cl₂ (c=0.05 M, calculated based on the reactant with 1.0 equivalent) was then added. The reaction system was pre-cooled to −78° C., and then TMSOTf (50 mol %) was added. After 30 minutes, acceptor IV-11 (27.9 mg, 0.052 mmol, 1.2 equivalent) was added. After the reaction was complete, a glycosylation product VI-16 was obtained through silica gel column chromatography separation and purification. The characterization data of this compound are as follows: ¹H NMR (600 MHz, CDCl₃) δ 7.97-7.93 (m, 2H), 7.92-7.88 (m, 2H), 7.81-7.77 (m, 2H), 7.54-7.48 (m, 2H), 7.43-7.26 (m, 17H), 7.24 (ddd, J=8.5, 4.1, 2.0 Hz, 2H), 7.18-7.13 (m, 2H), 5.86 (t, J=9.5 Hz, 1H), 5.49 (t, J=9.7 Hz, 1H), 5.37 (t, J=9.9 Hz, 1H), 5.02-4.96 (m, 1H), 4.80-4.74 (m, 3H), 4.66 (d, J=11.4 Hz, 1H), 4.53 (dd, J=11.4, 3.1 Hz, 2H), 4.46 (d, J=7.9 Hz, 1H), 4.42 (d, J=12.2 Hz, 1H), 4.05-3.98 (m, 2H), 3.73 (dd, J=11.4, 7.8 Hz, 1H), 3.71-3.61 (m, 4H), 3.47-3.41 (m, 1H), 2.84-2.70 (m, 2H), 2.02 (s, 3H), 1.27 (t, J=7.4 Hz, 3H). ¹³C NMR (151 MHz, CDCl₃) δ 169.69, 165.86, 165.52, 165.32, 138.30, 138.13, 138.00, 133.62, 133.40, 133.31, 130.01, 129.98, 129.83, 129.34, 129.01, 128.88, 128.57, 128.49, 128.40, 128.14, 127.99, 127.97, 127.89, 127.75, 101.25, 83.67, 83.03, 78.32, 77.95, 75.28, 75.19, 75.15, 74.35, 73.61, 73.16, 70.80, 69.81, 68.54, 68.52, 24.31, 21.15, 15.04.

### Example 46

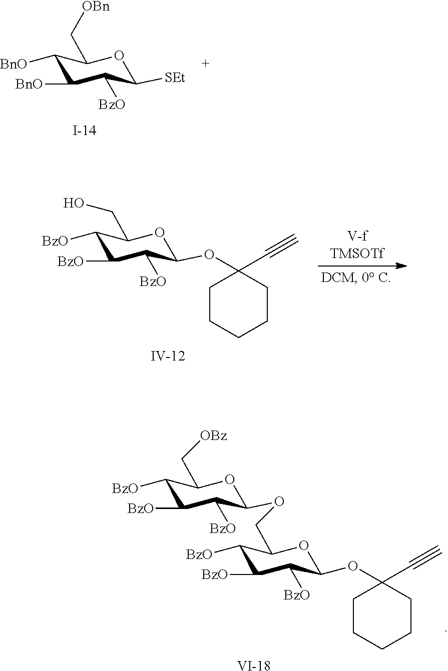

According to the reaction standard operating procedure, the raw materials were as follows: donor I-14 (25.7 mg, 0.04 mmol, 1.2 equiv), benziodoxole reagent V-f (9.7 mg, 0.013 mmol, 0.3 equiv), acceptor IV-12 (20.0 mg, 0.033 mmol, 1.0 equiv), and acid catalyst TMSOTf (20 mol %), and after the reaction was completed, the glycosylation product VI-18 (31.1 mg, 80%) was obtained. The characterization data of this compound are as follows: ¹H NMR (600 MHz, CDCl₃) δ 8.04-8.00 (m, 2H, Ar—H), 7.96-7.92 (m, 4H, Ar—H), 7.89-7.86 (m, 2H, Ar—H), 7.85-7.83 (m, 2H, Ar—H), 7.82-7.77 (m, 4H, Ar—H), 7.57-7.27 (m, 21H, Ar—H), 5.84 (t, J=9.6, 1H, H−3), 5.80 (t, J=9.6, 1H, H−3′), 5.60 (t, J=9.6 Hz, 1H, H−4′), 5.49 (dd, J=9.6, 7.8 Hz, 1H, H−2′), 5.43 (dd, J=9.6, 8.4 Hz, 1H, H−2), 5.32 (d, J=7.8 Hz, 1H, H−1), 5.31 (t, J=9.6 Hz, 1H, H−4), 5.05 (d, J=7.8 Hz, 1H, H−1′), 4.56 (dd, J=12.0, 3.0 Hz, 1H, H−6a′), 4.41 (dd, J=12.0, 4.8 Hz, 1H, H−6b′), 4.13-4.10 (m, 1H, H−5′), 4.09-4.05 (m, 1H, H−5), 4.00 (dd, J=11.4, 1.8 Hz, 1H, H−6a), 3.95 (dd, J=11.4, 7.2 Hz, 1H, H−6b), 2.52 (s, 1H, CH), 1.87-1.33 (m, 10H). ¹³C NMR (150 MHz, CDCl₃) δ 166.2, 165.9, 165.9, 165.6, 165.3, 165.2, 133.6, 133.4, 133.3, 133.3, 133.2, 133.1, 123.0, 129.9, 129.9, 129.9, 129.7, 129.4, 129.1, 128.9, 128.9, 128.9, 128.6, 128.5, 128.5, 128.5, 128.4, 128.4, 128.3, 100.8, 97.1, 84.2, 75.7, 74.3, 73.2, 73.1, 72.4, 72.0, 71.9, 70.1, 69.7, 68.3, 63.2, 38.4, 38.4, 25.0, 22.8. HRMS (ESI⁺): calc, for C₆₉H₆₀O₁₈ [M+Na]⁺1199.3672, found: 1199.3637.

### Example 47

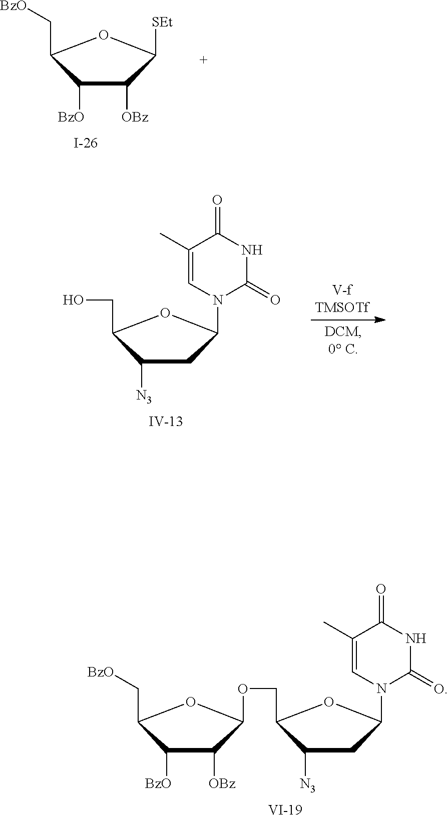

According to the reaction standard operating procedure, the raw materials were as follows: donor I-26 (22.7 mg, 0.045 mmol, 1.2 equiv), benziodoxole reagent V-f (8.5 mg, 0.011 mmol, 0.3 equiv), acceptor IV-13 (10.0 mg, 0.037 mmol, 1.0 equiv), and acid catalyst TMSOTf (20 mol %), and after the reaction was completed, the glycosylation product IV-13 (25.0 mg, 95%) was obtained. The characterization data of this compound are as follows: ¹H NMR (600 MHz, CDCl₃) δ 8.57 (s, 1H, NH), 8.02 (dd, J=8.4, 1.2 Hz, 2H, Ar—H), 7.98 (dd, J=8.4, 1.2 Hz, 2H, Ar—H), 7.90 (dd, J=8.4, 1.2 Hz, 2H, Ar—H), 7.59-7.55 (m, 1H, Ar—H), 7.54-7.50 (m, 2H, Ar—H), 7.42-7.32 (m, 6H, Ar—H), 7.27 (d, J=1.2 Hz, 1H, CH), 6.11 (t, J=6.6 Hz, 1H, H−1), 5.79 (dd, J=6.6, 5.4 Hz, 1H, H−3′), 5.68 (d, J=4.8 Hz, 1H, H−2′), 5.30 (s, 1H, H−1′), 4.77-4.71 (m, 2H, H−4, H−5′a), 4.56 (dd, J=11.4, 4.8 Hz, 1H, H−5′b), 4.16 (dt, J=7.8, 5.4 Hz, 1H, H−3), 4.04 (dd, J=11.4, 3.6 Hz, 1H, H−5a), 3.96-3.93 (m, 1H, H−4), 3.71 (dd, J=11.4, 4.0 Hz, 1H, H−5b), 2.35-2.25 (m, 2H, H−2), 1.89 (d, J=1.2 Hz, 3H, CH₃). ¹³C NMR (150 MHz, CDCl₃) δ 166.3, 165.5, 165.5, 163.5, 150.2, 135.5, 133.9, 133.7, 133.5, 129.9, 129.9, 129.8, 129.6, 129.0, 128.8, 128.7, 128.6, 128.6, 111.7, 106.5, 85.0, 82.7, 79.6, 75.3, 71.9, 67.6, 64.4, 60.5, 37.4, 12.7. HRMS (ESI⁺): calc, for C₃₆H₃₃N₅O₁₁ [M+Na]⁺734.2069, found: 734.2071.

### Example 48

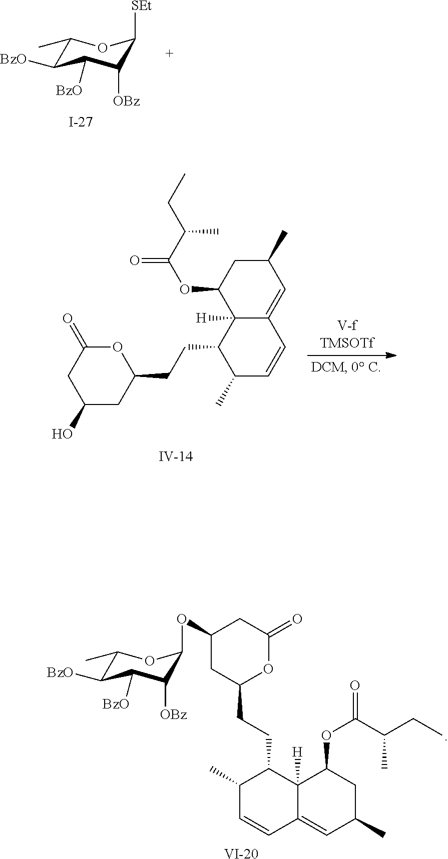

According to the reaction standard operating procedure, the raw materials were as follows: donor I-27 (20.0 mg, 0.052 mmol, 1.2 equiv), benziodoxole reagent V-f (7.2 mg, 0.010 mmol, 0.3 equiv), acceptor IV-14 (13.0 mg, 0.032 mmol, 1.0 equiv), and acid catalyst TMSOTf (20 mol %), and after the reaction was completed, the glycosylation product VI-20 (22.4 mg, 81%) was obtained. The characterization data of this compound are as follows: ¹H NMR (600 MHz, CDCl₃) δ 8.07 (d, J=7.2 Hz, 2H, Ar—H), 7.98 (d, J=7.2 Hz, 2H, Ar—H), 7.81 (d, J=7.2 Hz, 2H, Ar—H), 7.59 (t, J=7.2 Hz, 1H, Ar—H), 7.52-7.45 (m, 3H, Ar—H), 7.42-7.36 (m, 3H, Ar—H), 7.27-7.22 (m, 2H, Ar—H), 5.98 (d, J=9.6 Hz, 1H), 5.79 (dd, J=9.6, 6.0 Hz, 1H), 5.72 (dd, J=10.2, 3.0 Hz, 1H, H−3), 5.67 (t, J=9.6 Hz, 1H, H−4), 5.58 (dd, J=3.0, 1.8 Hz, 1H, H−2), 5.51 (s, 1H), 5.43-5.40 (m, 1H), 5.13 (s, 1H, H−1), 4.67-4.62 (m, 1H), 4.35-4.31 (m, 1H), 4.18-4.13 (m, 1H, H−5), 2.91 (dd, J=17.2, 3.6 Hz, 1H), 2.79 (dd, J=17.2, 5.4 Hz, 1H), 2.45-2.31 (m, 3H), 2.29-2.25 (m, 1H), 2.21-2.16 (m, 1H), 1.98 (dd, J=15.0, 3.6 Hz, 1H), 1.93-1.85 (m, 2H), 1.75-1.63 (m, 3H), 1.60-1.55 (m, 1H), 1.48-1.36 (m, 3H), 1.34 (d, J=6.0 Hz, 3H, CH₃-6), 1.11-1.05 (m, 6H, 2×CH₃), 0.93-0.87 (m, 6H, 2×CH₃). ¹³C NMR (150 MHz, CDCl₃) δ 176.7, 169.6, 165.9, 165.8, 165.5, 133.7, 133.5, 133.3, 133.2, 131.8, 130.0, 130.0, 129.8, 129.4, 129.2, 128.8, 128.6, 128.5, 128.4, 97.2, 76.7, 71.5, 71.1, 70.3, 69.9, 67.9, 67.7, 41.6, 37.5, 37.0, 36.8, 33.7, 33.1, 32.8, 30.9, 27.6, 27.0, 24.1, 23.0, 17.7, 16.4, 14.1, 11.9. HRMS (ESI⁺): calc. for C₅₁H₅₈O₁₂ [M+Na]⁺885.3820, found: 885.3803.

### Example 49

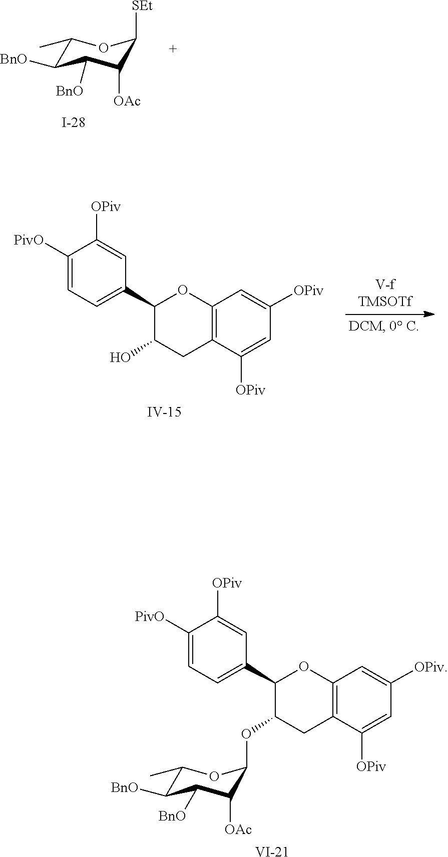

According to the reaction standard operating procedure, the raw materials were as follows: donor I-28 (20.0 mg, 0.047 mmol, 1.2 equiv), benziodoxole reagent V-f (8.8 mg, 0.012 mmol, 0.3 equiv), acceptor IV-15 (24.3 mg, 0.039 mmol, 1.0 equiv), and acid catalyst TMSOTf (20 mol %), and after the reaction was completed, the glycosylation product VI-21 (33.1 mg, 86%) was obtained. The characterization data of this compound are as follows: ¹H NMR (400 MHz, CDCl₃) δ 7.27-7.18 (m, 10H, Ar—H), 7.16-7.13 (m, 2H, Ar—H), 7.07-7.04 (m, 1H, Ar—H), 6.52 (d, J=2.0 Hz, 1H, Ar—H), 6.42 (d, J=2.0 Hz, 1H, Ar—H), 5.05 (dd, J=3.2, 1.6 Hz, 1H, H−2), 4.81-4.76 (m, 2H), 4.62 (d, J=11.2 Hz, 1H, —CH₂Ph), 4.49 (d, J=10.8 Hz, 1H, —CH₂Ph), 4.39 (d, J=11.2 Hz, 1H, —CH₂Ph), 4.12 (d, J=1.2 Hz, 1H, H−1), 3.91-3.85 (m, 1H), 3.74 (dd, J=9.2, 3.2 Hz, 1H, H−3), 3.70-3.63 (m, 1H, H−5), 3.26 (t, J=9.6 Hz, 1H, H−4), 2.79 (dd, J=16.0, 5.6 Hz, 1H), 2.61 (dd, J=16.0, 8.8 Hz, 1H), 1.98 (s, 3H, —COCH₃), 1.28 (s, 9H, C(CH₃)₃), 1.26 (s, 18H, 2×C(CH₃)₃), 1.25 (s, 9H, C(CH₃) 3), 1.19 (d, J=6.3 Hz, 3H, CH₃). ¹³C NMR (100 MHz, CDCl₃) δ 176.8, 176.2, 175.7, 175.6, 169.8, 154.9, 150.4, 149.7, 143.0, 142.8, 138.6, 138.3, 136.4, 128.5, 128.4, 128.3, 128.1, 127.8, 127.8, 124.9, 123.7, 121.5, 111.0, 108.8, 107.7, 98.6, 79.9, 78.9, 78.1, 75.5, 74.2, 71.9, 68.7, 68.1, 39.4, 39.3, 39.2, 27.4, 27.3, 27.2, 21.10, 18.0. HRMS (ESI⁺): calc. for C₅₇H₇₀O₁₅ [M+Na]⁺1017.4607, found: 1017.4609.

### Example 50

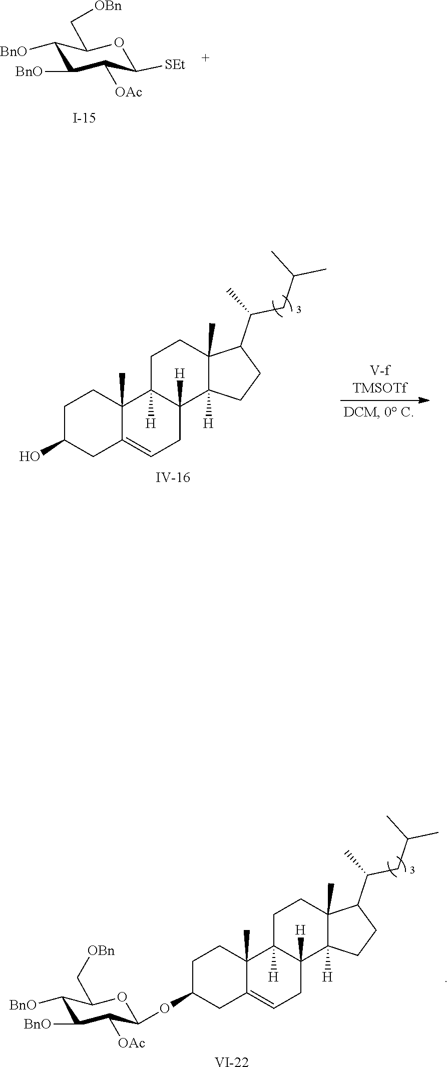

According to the reaction standard operating procedure, the raw materials were as follows: donor I-15 (20.0 mg, 0.037 mmol, 1.2 equiv), benziodoxole reagent V-f (7.0 mg, 0.009 mmol, 0.3 equiv), acceptor IV-16 (12.0 mg, 0.031 mmol, 1.0 equiv), and acid catalyst TMSOTf (20 mol %), and after the reaction was completed, the glycosylation product VI-22 (21.0 mg, 91%) was obtained. The characterization data of this compound are as follows: ¹H NMR (400 MHz, CDCl₃) δ 7.29-7.21 (m, 13H, Ar—H), 7.17-7.13 (m, 2H, Ar—H), 5.29 (d, J=4.8 Hz, 1H), 4.91 (dd, J=9.2, 8.8 Hz, 1H, H−2), 4.75 (d, J=11.2 Hz, 1H, —CH₂Ph), 4.74 (d, J=11.2 Hz, 1H, —CH₂Ph), 4.62 (d, J=11.2 Hz, 1H, —CH₂Ph), 4.57 (d, J=12.0 Hz, 1H, —CH₂Ph), 4.54-4.49 (m, 2H), 4.39 (d, J=8.0 Hz, 1H, H−1), 3.70 (dd, J=10.8, 2.0 Hz, 1H), 3.66-3.58 (m, 3H), 3.47-3.39 (m, 2H), 2.22-2.10 (m, 2H), 1.99-1.87 (m, 6H), 1.83-1.73 (m, 2H), 1.54-0.95 (m, 21H), 0.94 (s, 3H, —CH₃), 0.87 (d, J=6.4 Hz, 3H, —CH₃), 0.83 (d, J=1.2 Hz, 3H, —CH₃), 0.81 (d, J=1.2 Hz, 3H, —CH₃), 0.63 (s, 3H, —CH₃).

### Example 51

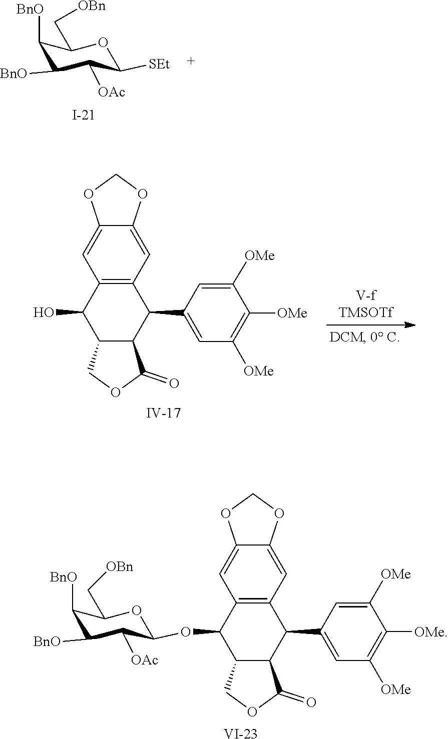

According to the reaction standard operating procedure, the raw materials were as follows: donor I-21 (27.9 mg, 0.052 mmol, 1.2 equiv), benziodoxole reagent V-f (9.7 mg, 0.013 mmol, 0.3 equiv), acceptor IV-17 (17.8 mg, 0.043 mmol, 1.0 equiv), and acid catalyst TMSOTf (20 mol %), and after the reaction was completed, the glycosylation product VI-23 (35.5 mg, 93%) was obtained. The characterization data of this compound are as follows: ¹H NMR (400 MHz, CDCl₃) δ 7.31-7.18 (m, 13H, Ar—H), 7.16-7.13 (m, 2H, Ar—H), 7.09 (s, 1H, Ar—H), 6.40 (s, 1H, Ar—H), 6.29 (s, 2H, Ar—H), 5.90 (d, J=1.2 Hz, 1H), 5.88 (d, J=1.2 Hz, 1H), 5.36 (dd, J=10.0, 8.0 Hz, 1H, H−2), 4.87 (d, J=11.6 Hz, 1H, —CH₂Ph), 4.80 (d, J=9.6 Hz, 1H), 4.61 (d, J=12.0 Hz, 1H, —CH₂Ph), 4.53 (d, J=11.6 Hz, 1H, —CH₂Ph), 4.50-4.46 (m, 2H), 4.43 (d, J=12.0 Hz, 1H, —CH₂Ph), 4.37-4.31 (m, 3H, H−1, 2×-CH₂Ph), 3.99 (dd, J=10.0, 9.2 Hz, 1H), 3.86 (d, J=2.4 Hz, 1H, H−4), 3.71 (s, 3H, —OCH₃), 3.66 (s, 6H, 2×-OCH₃), 3.57-3.49 (m, 2H, H−6a, H−6b), 3.47-3.41 (m, 2H, H−5, H−3), 2.85-3.75 (m, 1H), 2.69 (dd, J=14.4, 4.4 Hz, 1H), 1.94 (s, 3H, —COCH₃).

### Example 52

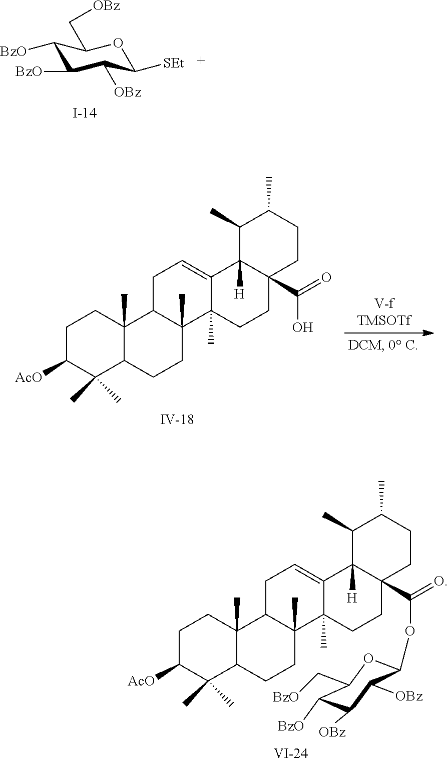

According to the reaction standard operating procedure, the raw materials were as follows: donor I-14 (33.3 mg, 0.052 mmol, 1.2 equiv), benziodoxole reagent V-f (9.7 mg, 0.013 mmol, 0.3 equiv), acceptor IV-18 (21.4 mg, 0.043 mmol, 1.0 equiv), and acid catalyst TMSOTf (20 mol %), and after the reaction was completed, the glycosylation product VI-24 (44.5 mg, 96%) was obtained. The characterization data of this compound are as follows: ¹H NMR (400 MHz, CDCl₃) δ 8.03-7.98 (m, 2H), 7.96-7.92 (m, 2H), 7.89-7.85 (m, 2H), 7.83-7.77 (m, 2H), 7.54-7.45 (m, 3H), 7.42-7.30 (m, 7H), 7.27 (d, J=7.9 Hz, 2H), 5.95 (t, J=9.7 Hz, 1H), 5.86 (d, J=8.4 Hz, 1H), 5.76-5.65 (m, 2H), 5.21 (t, J=3.2 Hz, 1H), 4.52 (dd, J=12.2, 3.0 Hz, 1H), 4.48-4.37 (m, 2H), 4.22 (ddd, J=9.8, 4.6, 3.2 Hz, 1H), 2.12 (d, J=11.4 Hz, 1H), 2.02 (s, 3H), 1.94-1.84 (m, 1H), 1.79 (dd, J=8.7, 3.2 Hz, 2H), 1.71 (dd, J=12.2, 4.6 Hz, 1H), 1.59 (d, J=12.0 Hz, 2H), 1.37 (ddd, J=27.3, 17.5, 6.5 Hz, 4H), 1.16 (dd, J=31.5, 8.3 Hz, 3H), 1.06-0.91 (m, 4H), 0.88 (s, 8H), 0.79 (t, J=5.1 Hz, 12H), 0.62 (t, J=11.0 Hz, 3H), 0.44 (s, 3H), 0.17 (d, J=12.2 Hz, 1H). ¹³C NMR (100 MHz, CDCl₃) δ 175.61, 171.14, 166.23, 165.83, 165.23, 164.91, 137.50, 133.63, 133.49, 133.46, 133.22, 130.11, 129.98, 129.90, 129.74, 128.92, 128.82, 128.58, 128.57, 128.48, 125.95, 92.11, 81.02, 72.97, 70.52, 69.40, 62.89, 55.30, 52.63, 48.26, 47.46, 41.99, 39.25, 39.10, 38.86, 38.35, 37.70, 36.85, 36.07, 32.14, 30.61, 28.34, 28.20, 24.02, 23.64, 23.33, 23.23, 21.46, 21.23, 18.21, 17.11, 16.88, 16.64, 15.59.

### Example 53

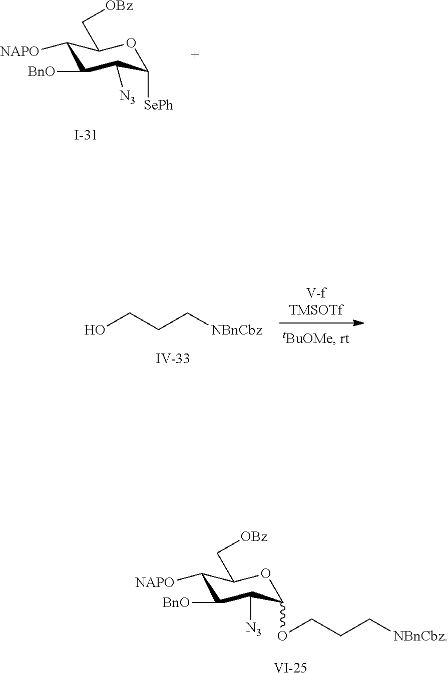

According to the reaction standard operating procedure, the raw materials were as follows: donor I-31 (34.0 mg, 0.050 mmol, 1.5 equiv), benziodoxole reagent V-f (10.1 mg, 0.013 mmol, 0.4 equiv), acceptor IV-33 (10.0 mg, 0.033 mmol, 1.0 equiv), and acid catalyst TMSOTf (20 mol %), and after the reaction was completed, the glycosylation product VI-25 (23.8 mg, 88%) was obtained. α configuration: ¹H NMR (600 MHz, CDCl₃) δ 7.91 (d, J=7.2 Hz, 2H), 7.76 (d, J=7.8 Hz, 2H), 7.71 (dd, J=6.0, 3.4 Hz, 1H), 7.66 (s, 1H), 7.51 (t, J=7.4 Hz, 1H), 7.45-7.13 (m, 20H), 5.17 (d, J=17.4 Hz, 2H), 5.01 (d, J=11.1 Hz, 1H), 4.95-4.73 (m, 4H), 4.60-4.40 (m, 4H), 4.08-3.86 (m, 2H), 3.77-3.58 (m, 2H), 3.52-3.27 (m, 4H), 1.84 (dd, J=36.3, 29.8 Hz, 2H). β configuration: ¹H NMR (600 MHz, CDCl₃) δ 7.90 (d, J=5.8 Hz, 2H), 7.76-7.69 (m, 3H), 7.63 (s, 1H), 7.49 (t, J=7.5 Hz, 1H), 7.44-7.39 (m, 2H), 7.40-7.24 (m, 14H), 7.24-7.08 (m, 4H), 5.14 (d, J=16.6 Hz, 2H), 4.99 (d, J=11.0 Hz, 1H), 4.94 (d, J=10.5 Hz, 1H), 4.82 (d, J=10.6 Hz, 1H), 4.76 (d, J=11.0 Hz, 1H), 4.61 (dd, J=18.4, 7.2 Hz, 1H), 4.49 (dt, J=26.1, 7.6 Hz, 3H), 4.23 (dd, J=55.8, 7.0 Hz, 1H), 3.95-3.79 (m, 1H), 3.69 (t, J=9.0 Hz, 1H), 3.63-3.49 (m, 2H), 3.48-3.24 (m, 4H), 1.84 (m, 2H).

Unless otherwise specified, the standard operating procedure for the alkylation reaction in the examples is as follows: the alkylation reagent (II) or alkylation reagent (III) as described in the disclosure, benziodoxole reagent, acceptor, and dried molecular sieves 4 Å MS were placed in a reaction flask and sealed with a rubber stopper, and anhydrous CH₂Cl₂ (c=0.05 M, calculated based on the reactant with 1.0 equivalent) was then added. The reaction system was pre-cooled to 0° C., and then acid catalyst (20 mol %) was added. After the reaction was complete, a glycosylation product was obtained through separation and purification by silica gel column chromatography. In some implementation examples, where there are special instructions that are inconsistent with the standard operating procedure for the reaction, the special instructions shall prevail.

### Example 54

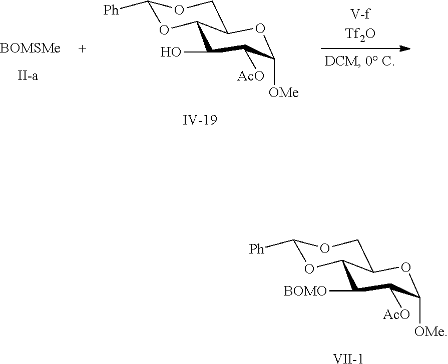

According to the reaction standard operating procedure, the raw materials were as follows: sulfide II-a (18.68 mg, 0.11 mmol, 1.2 equiv), benziodoxole reagent V-f (20.93 mg, 0.028 mmol, 0.3 equiv), acceptor IV-19 (30.0 mg, 0.092 mmol, 1.0 equiv), acid catalyst Tf2O (5 mol %), and after the reaction was complete, the target product VII-1 (40.0 mg, 97%) was obtained. The characterization data of this compound are as follows: ¹H NMR (400 MHz, CDCl₃) δ 7.40-7.35 (m, 2H, Ar—H), 7.26-7.11 (m, 8H, Ar—H), 5.48 (s, 1H, Ph-H), 4.88 (d, J=1.6, 5.2 Hz, 2H, OCH₂O), 4.82-4.76 (m, 2H, H−1, H−2), 4.52 (dd, J=11.6, 13.6 Hz, 2H, Bn-H), 4.28-4.19 (m, 2H, H−6, H−3), 3.80 (td, J=4.8, 10 Hz, 1H, H−5), 3.69 (t, J=10 Hz, 1H, H−6), 3.59 (t, J=9.6 Hz, 1H, H−4), 3.32 (s, 3H, OMe), 2.00 (s, 3H, OAc). ¹³C NMR (100 MHz, CDCl₃) δ 192.5, 170.5, 137.9, 137.3, 134.6, 129.9, 129.2, 129.1, 128.7, 128.4, 128.3, 128.3, 128.1, 127.9, 127.6, 126.3, 101.8, 97.7, 97.0, 96.3, 95.1, 82.5, 81.5, 73.4, 72.6, 71.8, 71.0, 70.6, 70.4, 69.5, 69.1, 62.7, 62.4, 55.4, 55.3, 21.1.

### Example 55

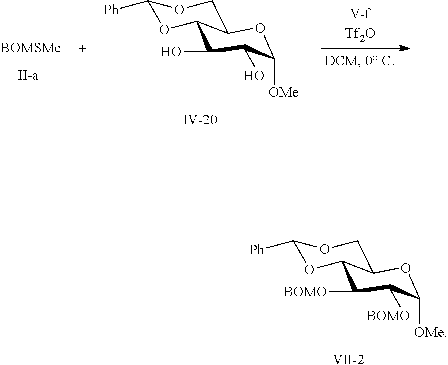

According to the reaction standard operating procedure, the raw materials were as follows: sulfide II-a (28.61 mg, 0.17 mmol, 2.4 equiv), benziodoxole reagent V-f (32.06 mg, 0.043 mmol, 0.6 equiv), acceptor IV-20 (20.0 mg, 0.071 mmol, 1.0 equiv), acid catalyst Tf2O (10 mol %), and after the reaction was complete, the target product VII-2 (31.7 mg, 86%) was obtained. The characterization data of this compound are as follows: ¹H NMR (400 MHz, CDCl₃) δ 7.37-7.33 (m, 2H, Ar—H), 7.28-7.05 (m, 13H, Ar—H), 5.44 (s, 1H, PhCH), 4.88-4.84 (m, 3H, BnOCH₂O), 4.82 (t, J=6.4 Hz, 1H, BnOCH₂O), 4.75 (d, J=3.6 Hz, 1H, H−1), 4.64 (d, J=12.0 Hz, 1H, Bn-H), 4.55 (d, J=12.0 Hz, 1H, Bn-H), 4.49 (t, J=12.0 Hz, 2H, Bn-H), 4.18 (dd, J=4.8, 10.0 Hz, 1H, H−6a), 4.13 (t, J=9.2 Hz, 1H, H−4), 3.76 (td, J=4.8, 10 Hz, 1H, H−5), 3.68-3.60 (m, 2H, H−6b, H−2), 3.50 (t, J=9.6 Hz, 1H, H−3), 3.30 (s, 3H, OCH₃). ¹³C NMR (101 MHz, CDCl₃) δ 138.0, 137.7, 137.4, 129.1, 128.6, 128.4, 128.4, 128.0, 127.9, 127.8, 127.6, 126.3, 101.9, 99.9, 96.0, 95.5, 81.7, 79.0, 74.3, 69.8, 69.6, 69.2, 62.5, 55.4.

### Example 56

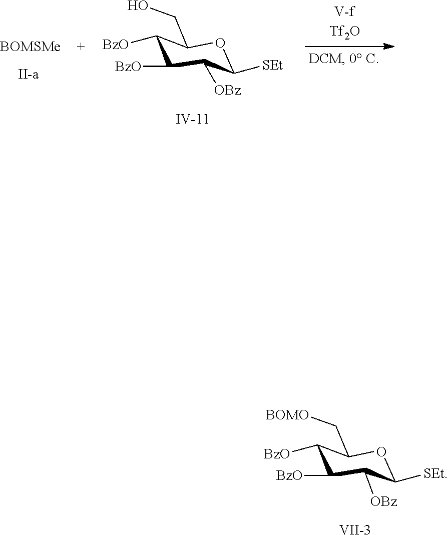

According to the reaction standard operating procedure, the raw materials were as follows: sulfide II-a (14.12 mg, 0.084 mmol, 1.5 equiv), benziodoxole reagent V-f (16.87 mg, 0.022 mmol, 0.4 equiv), acceptor IV-11 (30.0 mg, 0.056 mmol, 1.0 equiv), acid catalyst Tf2O (5 mol %), and after the reaction was complete, the target product VII-3 (34.5 mg, 94%) was obtained. The characterization data of this compound are as follows: ¹H NMR (400 MHz, CDCl₃) δ 7.95-7.88 (m, 4H, Ar—H), 7.82-7.77 (m, 2H, Ar—H), 7.52-7.46 (m, 2H, Ar—H), 7.43-7.32 (m, 5H, Ar—H), 7.28-7.21 (m, 7H, Ar—H), 5.86 (t, J=9.2 Hz, 1H, H−3), 5.60 (t, J=10.0 Hz, 1H, H−4), 5.53 (t, J=10.0 Hz, 1H, H−2), 4.78 (d, J=10.0 Hz, 1H, H−1), 4.76 (d, J=6.8 Hz, 1H, BnOCH₂O), 4.73 (d, J=6.8 Hz, 1H, BnOCH₂O), 4.53 (s, 2H, Bn-H), 3.97 (td, J=3.6, 8 Hz, 1H, H−5), 3.85-3.77 (m, 2H, H−6a, H−6b), 2.83 (m, 2H, SCH₂CH₃), 1.25 (t, J=7.6 Hz, 3H, SCH₂CH₃). ¹³C NMR (101 MHz, CDCl₃) δ 166.0, 165.4, 165.3, 137.8, 133.5, 133.4, 133.3, 130.0, 129.9, 129.9, 129.4, 129.2, 129.1, 128.6, 128.5, 128.4, 128.1, 127.8, 95.0, 83.9, 77.9, 74.6, 70.8, 69.7, 69.5, 66.8, 24.2, 15.0.

### Example 57

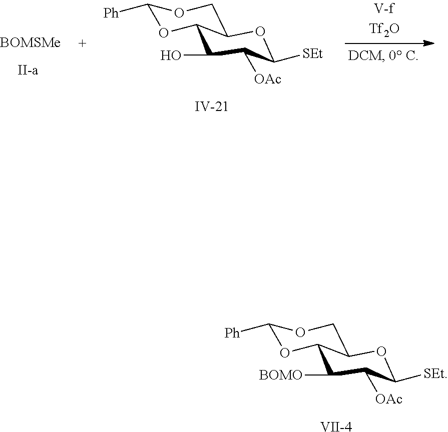

According to the reaction standard operating procedure, the raw materials were as follows: sulfide II-a (11.40 mg, 0.068 mmol, 1.2 equiv), benziodoxole reagent V-f (12.77 mg, 0.017 mmol, 0.3 equiv), acceptor IV-21 (20.0 mg, 0.056 mmol, 1.0 equiv), acid catalyst Tf2O (5 mol %), and after the reaction was complete, the target product VII-4 (25.4 mg, 95%) was obtained. The characterization data of this compound are as follows: ¹H NMR (600 MHz, CDCl₃) δ 7.45-7.41 (m, 2H, Ar—H), 7.33-7.19 (m, 8H, Ar—H), 5.54 (s, 1H, PhCH), 5.07 (t, J=9.0 Hz, 1H, H−2), 4.97 (d, J=6.6 Hz, 1H, BnOCH₂O), 4.81 (d, J=6.6 Hz, 1H, BnOCH₂O), 4.57 (d, J=11.4 Hz, 1H, Bn-H), 4.52 (d, J=11.4 Hz, 1H, Bn-H), 4.49 (d, J=10.2 Hz, 1H, H−1), 4.36 (dd, J=5.4, 10.8 Hz, 1H, H−6a), 4.05 (t, J=9.0 Hz, 1H, H−3), 3.76 (t, J=10.2 Hz, 1H, H−6b), 3.69 (t, J=9.6 Hz, 1H, H−4), 3.51 (td, J=4.8, 9.6 Hz, 1H, H−5), 2.74-2.66 (m, 2H, SCH₂CH₃), 2.02 (s, 3H, OAc), 1.25 (t, J=7.2 Hz, 3H, SCH₂CH₃). ¹³C NMR (150 MHz, CDCl₃) δ 169.8, 137.7, 137.1, 129.2, 128.5, 128.4, 127.8, 127.8, 126.2, 101.7, 95.0, 84.5, 81.0, 77.0, 71.5, 70.8, 69.6, 68.7, 24.2, 21.1, 14.9.

### Example 58

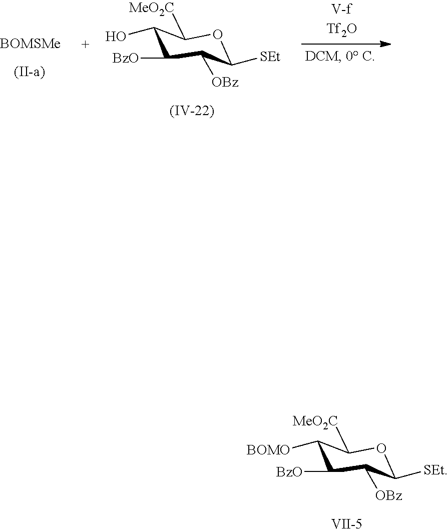

According to the reaction standard operating procedure, the raw materials were as follows: sulfide II-a (13.16 mg, 0.078 mmol, 1.2 equiv), benziodoxole reagent V-f (14.74 mg, 0.020 mmol, 0.3 equiv), acceptor IV-22 (30.0 mg, 0.065 mmol, 1.0 equiv), acid catalyst Tf2O (5 mol %), and after the reaction was complete, the target product VII-5 (27.5 mg, 73%) was obtained. The characterization data of this compound are as follows: ¹H NMR (400 MHz, CDCl₃) δ 7.97-7.91 (m, 4H, Ar—H), 7.55-7.46 (m, 2H, Ar—H), 7.41-7.30 (m, 4H, Ar—H), 7.29-7.21 (m, 3H, Ar—H), 7.16-7.11 (m, 2H, Ar—H), 5.75 (t, J=9.6 Hz, 1H, H−3), 5.46 (t, J=10.0 Hz, 1H, H−2), 4.79 (d, J=10.0 Hz, 1H, H−1), 4.75 (d, J=6.8 Hz, 1H, BnOCH₂), 4.70 (d, J=6.8 Hz, 1H, BnOCH₂), 4.46 (d, J=12.0 Hz, 1H, Bn-H), 4.33 (d, J=12.8 Hz, 1H, Bn-H), 4.32 (t, J=9.6 Hz, 1H, H−4), 4.19 (d, J=9.6 Hz, 1H, H−5), 3.76 (s, 3H, COOCH₃), 2.83-2.73 (m, 2H, SCH₂CH₃), 1.27 (t, J=7.6 Hz, 3H, SCH₂CH₃). ¹³C NMR (150 MHz, CDCl₃) δ 168.0, 165.8, 165.4, 137.3, 133.4, 133.4, 130.0, 129.9, 129.3, 129.2, 128.5, 128.5, 128.4, 127.7, 95.6, 84.3, 78.9, 76.6, 75.2, 70.4, 70.3, 52.9, 24.4, 14.8.

### Example 59

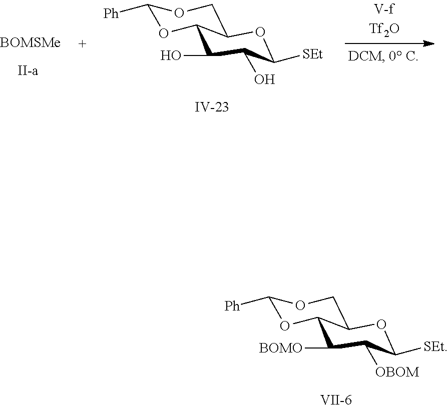

According to the reaction standard operating procedure, the raw materials were as follows: sulfide II-a (53.86 mg, 0.32 mmol, 5.0 equiv), benziodoxole reagent V-f (24.14 mg, 0.032 mmol, 0.5 equiv), acceptor IV-23 (20.0 mg, 0.064 mmol, 1.0 equiv), acid catalyst Tf2O (10 mol %), and after the reaction was complete, the target product VII-6 (26.3 mg, 74%) was obtained. The characterization data of this compound are as follows: ¹H NMR (400 MHz, CDCl₃) δ 7.39-7.35 (m, 2H, Ar—H), 7.31-7.15 (m, 11H, Ar—H), 7.12-7.07 (m, 2H, Ar—H), 5.46 (s, 1H, Ph-H), 5.02 (d, J=6.8 Hz, 1H, BnOCH₂O), 4.97-4.89 (m, 3H, BnOCH₂O+Bn-H), 4.79 (d, J=11.6 Hz, 1H, Bn-H), 4.63 (d, J=12 Hz, 1H, Bn-H), 4.53 (s, 2H, BnOCH₂O), 4.48 (d, J=9.6 Hz, 1H, H−1), 4.260 (dd, J=4.4, 10 Hz, 1H, H−6), 3.94 (t, J=8.8 Hz, 1H, H−3), 3.69 (t, J=10.4 Hz, 1H, H−6), 3.59 (q, J=9.6, 18.4 Hz, 2H, H−2, H−4), 3.42 (td, J=4.8, 9.6 Hz, 1H, H−5), 2.72-2.63 (m, 2H, SCH₂CH₃), 1.21 (t, J=7.2 Hz, 3H, SCH₂CH₃). ¹³C NMR (100 MHz, CDCl₃) δ 138.38, 138.26, 137.63, 130.28, 129.59, 129.53, 129.20, 128.90, 128.84, 128.79, 128.58, 128.37, 128.29, 128.17, 128.07, 126.70, 102.13, 96.71, 96.06, 86.06, 81.36, 79.55, 78.33, 71.27, 70.98, 70.57, 69.25, 25.16, 15.36.

### Example 60

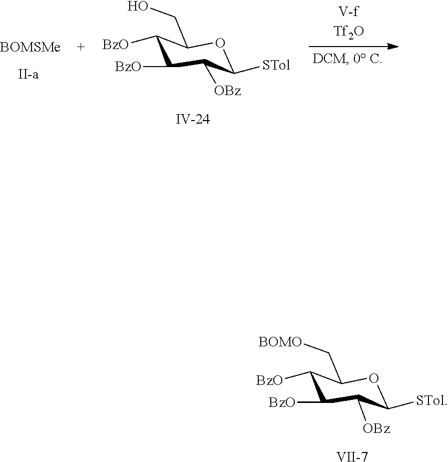

According to the reaction standard operating procedure, the raw materials were as follows: sulfide II-a (10.12 mg, 0.060 mmol, 1.2 equiv), benziodoxole reagent V-f (11.34 mg, 0.015 mmol, 0.3 equiv), acceptor IV-24 (30.0 mg, 0.050 mmol, 1.0 equiv), acid catalyst Tf2O (5 mol %), and after the reaction was complete, the target product VII-7 (26.9 mg, 74%) was obtained. The characterization data of this compound are as follows: ¹H NMR (400 MHz, CDCl₃) δ 7.97-7.92 (m, 2H, Ar—H), 7.90-7.85 (m, 2H, Ar—H), 7.79-7.74 (m, 2H, Ar—H), 7.52-7.45 (m, 2H, Ar—H), 7.40-7.30 (m, 7H, Ar—H), 7.27-7.21 (m, 7H, Ar—H), 7.07 (d, J=8.0 Hz, 2H, Ar—H), 5.83 (t, J=9.2 Hz, 1H, H−3), 5.53 (t, J=9.6 Hz, 1H, H−4), 5.42 (t, J=9.6 Hz, 1H, H−2), 4.94 (d, J=10.0 Hz, 1H, H−1), 4.77-4.71 (q, J=6.8, 10.0 Hz, 2H, BnOCH₂), 4.53 (t, J=12.4 Hz, 2H, Bn-H), 3.96 (m, 1H, H−5), 3.84-3.75 (m, 2H, H−6a, H−6b), 2.31 (s, 3H, CH₃). ¹³C NMR (150 MHz, CDCl₃) δ 166.0, 165.3, 165.2, 138.7, 137.8, 133.8, 133.5, 133.4, 133.3, 130.0, 129.9, 129.9, 129.9, 129.5, 129.09, 129.0, 128.6, 128.5, 128.4, 128.1, 127.8, 95.0, 86.4, 77.9, 74.6, 70.8, 69.6, 69.5, 66.8, 21.3.

### Example 61

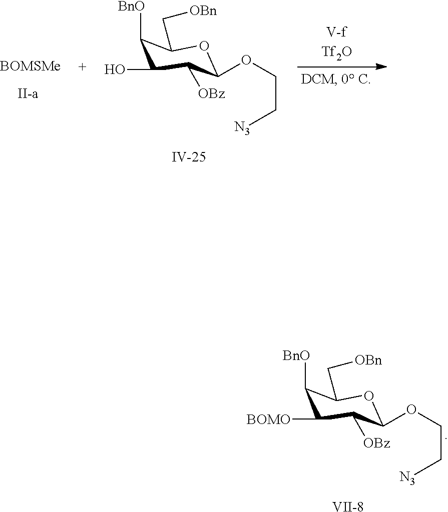

According to the reaction standard operating procedure, the raw materials were as follows: sulfide II-a (13.25 mg, 0.079 mmol, 1.2 equiv), benziodoxole reagent V-f (14.84 mg, 0.020 mmol, 0.3 equiv), acceptor IV-25 (35.0 mg, 0.066 mmol, 1.0 equiv), acid catalyst Tf2O (5 mol %), and after the reaction was complete, the target product VII-8 (34.2 mg, 80%) was obtained. The characterization data of this compound are as follows: ¹H NMR (600 MHz, CDCl₃) δ 8.07-8.03 (m, 2H, Ar—H), 7.54-7.50 (m, 1H, Ar—H), 7.40-7.36 (m, 2H, Ar—H), 7.35-7.20 (m, 13H, Ar—H), 7.10-7.05 (m, 2H, Ar—H), 5.66 (dd, J=8.4, 10.2 Hz, 1H, H−2), 4.92 (d, J=11.4 Hz, 1H, Bn-H), 4.74 (d, J=6.6 Hz, 1H, BnOCH₂), 4.67 (d, J=7.2 Hz, 1H, BnOCH₂), 4.61 (d, J=8.4 Hz, 1H, H−1), 4.60 (d, J=12.0 Hz, 1H, Bn-H), 4.48 (d, J=12.0 Hz, 1H, Bn-H), 4.44 (d, J=12.0 Hz, 2H, Bn-H), 4.39 (d, J=12.0 Hz, 1H, Bn-H), 4.01 (dd, J=3.0, 10.2 Hz, 1H, H−3), 3.99-3.95 (m, 2H, H−4. H−5), 3.70 (t, J=6.6 Hz, 1H, OCH₂CH₂N₃), 3.67-3.61 (m, 3H, OCH₂CH₂N₃), 3.37 (m, 1H, H−6a), 3.24 (dt, J=4.8, 13.2 Hz, 1H, H−6b). ¹³C NMR (150 MHz, CDCl₃) δ 165.5, 138.4, 137.9, 137.5, 133.2, 130.1, 129.9, 128.6, 128.5, 128.4, 128.4, 128.0, 127.8, 127.6, 101.6, 93.2, 77.2, 74.7, 74.0, 73.7, 73.5, 71.4, 69.8, 68.6, 67.6, 50.8.

### Example 62

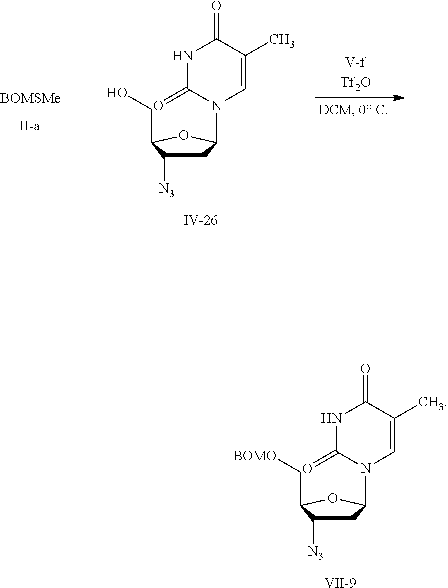

According to the reaction standard operating procedure, the raw materials were as follows: sulfide II-a (15.11 mg, 0.090 mmol, 1.2 equiv), benziodoxole reagent V-f (16.93 mg, 0.022 mmol, 0.3 equiv), acceptor IV-26 (20.0 mg, 0.075 mmol, 1.0 equiv), acid catalyst Tf2O (5 mol %), and after the reaction was complete, the target product VII-9 (25.6 mg, 88%) was obtained. The characterization data of this compound are as follows: ¹H NMR (600 MHz, CDCl₃) δ 8.88 (s, 1H, NH), 7.48 (s, 1H, NCHC), 7.38-7.28 (m, 5H, Ar—H), 6.19 (t, J=6.0 Hz, 1H, H−1), 4.84 (s, 2H, BnOCH₂), 4.63 (d, J=12.0 Hz, 1H, Bn-H), 4.60 (d, J=12.0 Hz, 1H, Bn-H), 4.11 (dt, J=5.4, 10.2 Hz, 1H, H−3), 4.00 (dt, J=3.0, 6.0 Hz, 1H, H−4), 3.86 (dd, J=3.0, 10.8 Hz, 1H, H−5a), 3.78 (dd, J=3.0, 10.8 Hz, 1H, H−5b), 2.39 (dt, J=6.0, 14.4 Hz, 1H, H−6a), 2.26 (dt, J=6.0, 14.4 Hz, 1H, H−6b), 1.88 (s, 3H, CH₃). ¹³C NMR (150 MHz, CDCl₃) δ 163.8, 150.3, 137.5, 135.6, 128.7, 128.2, 127.9, 111.1, 95.4, 84.9, 83.2, 70.5, 67.2, 60.4, 38.0, 12.8.

### Example 63

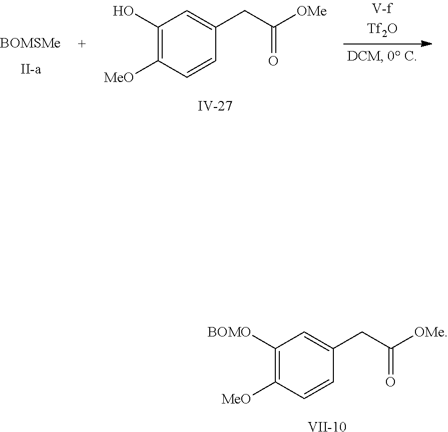

According to the reaction standard operating procedure, the raw materials were as follows: sulfide II-a (25.73 mg, 0.15 mmol, 1.5 equiv), benziodoxole reagent V-f (30.75 mg, 0.041 mmol, 0.4 equiv), acceptor IV-27 (20.0 mg, 0.10 mmol, 1.0 equiv), acid catalyst Tf2O (5 mol %), and after the reaction was complete, the target product VII-10 (26.8 mg, 83%) was obtained. The characterization data of this compound are as follows: ¹H NMR (400 MHz, CDCl₃) δ 7.26-7.16 (m, 5H, Ar—H), 7.06 (d, J=2.0 Hz, 1H, Ar—H), 6.82 (dd, J=2.4, 8.4 Hz, 1H, Ar—H), 6.76 (d, J=8.0 Hz, 1H, Ar—H), 5.26 (s, 2H, PhCH₂OCH₂), 4.69 (s, 2H, PhCH₂OCH₂), 3.78 (s, 3H, COOCH₃), 3.57 (s, 3H, OCH₃), 3.46 (s, 2H, PhCH₂CO₂Me). ¹³C NMR (100 MHz, CDCl₃) δ 172.3, 149.1, 146.6, 137.5, 128.5, 128.1, 127.9, 126.7, 123.4, 117.9, 111.9, 93.7, 70.3, 56.1, 52.1, 40.6.

### Example 64

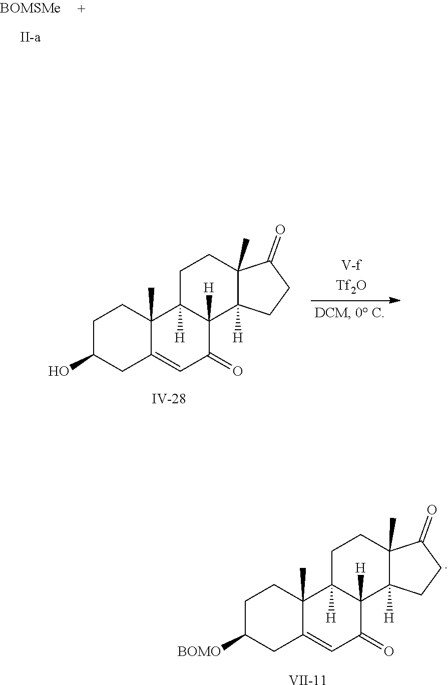

According to the reaction standard operating procedure, the raw materials were as follows: sulfide II-a (13.36 mg, 0.079 mmol, 1.2 equiv), benziodoxole reagent V-f (14.96 mg, 0.020 mmol, 0.3 equiv), acceptor IV-28 (20.0 mg, 0.066 mmol, 1.0 equiv), acid catalyst Tf2O (5 mol %), and after the reaction was complete, the target product VII-11 (26.9 mg, 96%) was obtained. The characterization data of this compound are as follows: ¹H NMR (600 MHz, CDCl₃) δ 7.36-7.25 (m, 5H), 5.70 (d, J=2.4 Hz, 1H), 4.82 (d, J=7.2 Hz, 1H), 4.80 (d, J=7.2 Hz, 1H), 4.61 (s, 2H), 3.64-3.57 (m, 1H), 2.83-2.76 (m, 1H), 2.54 (ddd, J=1.8, 4.8, 13.8 Hz, 1H), 2.47-2.35 (m, 3H), 2.15-2.07 (m, 1H), 2.03-1.97 (m, 1H), 1.95 (dt, J=4.2, 13.8 Hz, 1H), 1.83 (dt, J=3.0, 13.2 Hz, 1H), 1.77-1.70 (m, 2H), 1.66-1.61 (m, 2H), 1.55-1.52 (m, 1H), 1.24-1.15 (m, 6H), 0.87 (s, 3H).

### Example 65

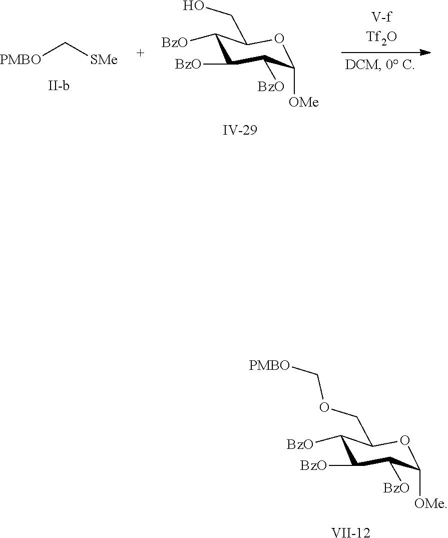

According to the reaction standard operating procedure, the raw materials were as follows: sulfide II-a (9.40 mg, 0.047 mmol, 1.2 equiv), benziodoxole reagent V-f (8.94 mg, 0.012 mmol, 0.3 equiv), acceptor IV-29 (20.0 mg, 0.039 mmol, 1.0 equiv), acid catalyst Tf2O (5 mol %), and after the reaction was complete, the target product VII-12 (25.4 mg, 98%) was obtained. The characterization data of this compound are as follows: ¹H NMR (400 MHz, CDCl₃) δ 7.87-7.83 (m, 2H, Ar—H), 7.82-7.78 (m, 2H, Ar—H), 7.76-7.72 (m, 2H, Ar—H), 7.41-7.34 (m, 2H, Ar—H), 7.32-7.21 (m, 5H, Ar—H), 7.18-7.11 (m, 2H, Ar—H), 7.05-7.00 (m, 2H, Ar—H), 6.67-6.62 (m, 2H, Ar—H), 6.01 (t, J=9.6 Hz, 1H, H−3), 5.56 (t, J=9.6 Hz, 1H, H−4), 5.16 (dd, J=3.6, 9.6 Hz, 1H, H−2), 5.13 (d, J=3.6 Hz, 1H, H−1), 4.63 (q, J=6.8, 9.2 Hz, 2H, OCH₂O), 4.36 (s, 2H, PhCH₂O), 4.11 (ddd, J=2.8, 4.4, 10.4 Hz, 1H, H−5), 3.72-3.65 (m, 2H, H−6a, H−6b), 3.64 (s, 3H, PhOCH₃), 3.36 (s, 3H, —OCH₃). ¹³C NMR (150 MHz, CDCl₃) δ 166.0, 165.4, 133.5, 133.2, 130.1, 130.0, 129.8, 129.7, 129.4, 129.2, 128.6, 128.6, 128.4, 113.9, 97.3, 94.8, 72.3, 70.8, 69.4, 69.2, 68.8, 66.1, 55.8.

### Example 66

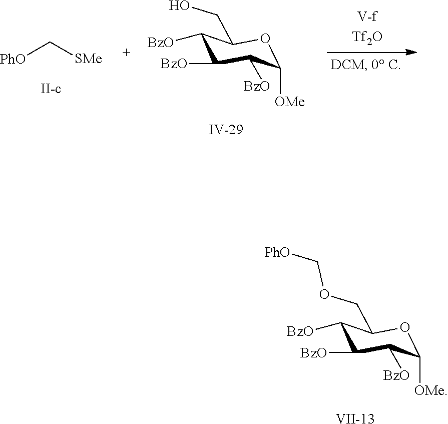

According to the reaction standard operating procedure, the raw materials were as follows: sulfide II-c (10.97 mg, 0.071 mmol, 1.2 equiv), benziodoxole reagent V-f (13.40 mg, 0.018 mmol, 0.3 equiv), acceptor IV-29 (30.0 mg, 0.059 mmol, 1.0 equiv), acid catalyst Tf2O (5 mol %), and after the reaction was complete, the target product VII-13 (35.3 mg, 97%) was obtained. The characterization data of this compound are as follows: ¹H NMR (400 MHz, CDCl₃) δ 7.87-7.82 (m, 2H), 7.78-7.71 (m, 4H), 7.40-7.34 (m, 2H), 7.32-7.20 (m, 4H), 7.19-7.06 (m, 4H), 6.88-6.80 (m, 3H), 5.99 (t, J=9.6 Hz, 1H, H−3), 5.45 (t, J=10.0 Hz, 1H, H−4), 5.17 (d, J=6.8 Hz, 1H, OCH₂O), 5.13 (dd, J=3.6, 10.0 Hz, 1H, H−2), 5.09 (m, 2H, H−1, OCH₂O), 4.10 (dt, J=4.0, 10.0 Hz, 1H, H−5), 3.80-3.72 (m, 2H, H−6a, H−6b), 3.30 (s, 3H, OMe). ¹³C NMR (150 MHz, CDCl₃) δ 166.0, 165.9, 165.4, 157.2, 133.5, 133.5, 133.2, 130.1, 129.9, 129.8, 129.6, 129.4, 129.2, 129.1, 128.5, 128.5, 128.4, 122.1, 116.4, 116.3, 97.1, 93.9, 72.2, 70.7, 69.6, 68.9, 67.4, 55.7.

### Example 67

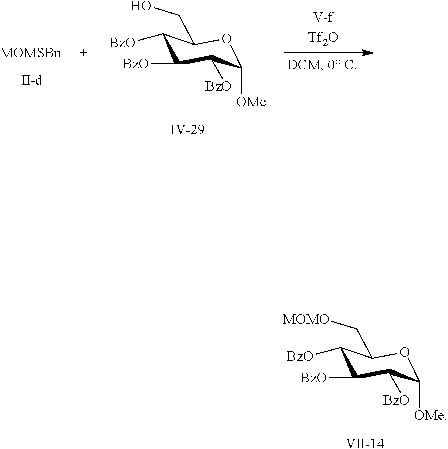

According to the reaction standard operating procedure, the raw materials were as follows: sulfide II-d (11.96 mg, 0.071 mmol, 1.2 equiv), benziodoxole reagent V-f (13.40 mg, 0.018 mmol, 0.3 equiv), acceptor II-30 (30.0 mg, 0.059 mmol, 1.0 equiv), acid catalyst Tf2O (5 mol %), and after the reaction was complete, the target product VII-14 (28.9 mg, 89%) was obtained. The characterization data of this compound are as follows: ¹H NMR (400 MHz, CDCl₃) δ 7.86-7.79 (m, 4H, Ar—H), 7.76-7.71 (m, 2H, Ar—H), 7.41-7.34 (m, 2H, Ar—H), 7.31-7.11 (m, 7H, Ar—H), 6.00 (t, J=9.6 Hz, 1H, H−3), 5.52 (t, J=10.0 Hz, 1H, H−4), 5.14 (dd, J=4, 9.6 Hz, 1H, H−2), 5.12 (d, J=4 Hz, 1H, H−1), 4.52 (q, J=6.4, 15.2 Hz, 2H, H−6a, H−6b), 4.09 (dt, J=4.0, 10.8 Hz, 1H, H−5), 3.61 (d, J=4 Hz, 2H, OCH₂O), 3.35 (s, 3H, OCH₃), 3.19 (s, 3H, —CH₂OCH₃). ¹³C NMR (150 MHz, CDCl₃) δ 166.0, 165.4, 133.5, 133.5, 133.2, 130.1, 129.9, 129.8, 129.4, 129.2, 128.6, 128.5, 128.4, 97.2, 97.0, 72.3, 70.8, 69.4, 68.8, 66.0, 55.8, 55.5.

### Example 68

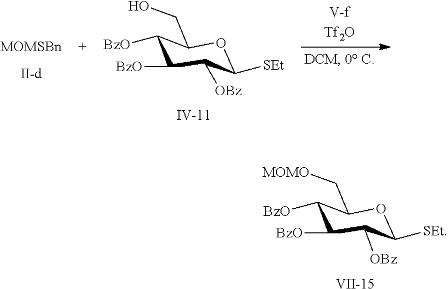

According to the reaction standard operating procedure, the raw materials were as follows: sulfide II-d (14.14 mg, 0.084 mmol, 1.5 equiv), benziodoxole reagent V-f (16.87 mg, 0.022 mmol, 0.4 equiv), acceptor IV-11 (30.0 mg, 0.056 mmol, 1.0 equiv), acid catalyst Tf2O (5 mol %), and after the reaction was complete, the target product VII-15 (24.3 mg, 75%) was obtained. The characterization data of this compound are as follows: ¹H NMR (400 MHz, CDCl₃) δ 7.95-7.88 (m, 4H, Ar—H), 7.81-7.77 (m, 2H, Ar—H), 7.52-7.46 (m, 2H, Ar—H), 7.43-7.22 (m, 7H, Ar—H), 5.86 (t, J=9.2 Hz, 1H, H−3), 5.56 (t, J=9.6 Hz, 1H, H−4), 5.52 (t, J=9.6 Hz, 1H, H−2), 4.79 (d, J=10.0 Hz, 1H, H−1), 4.61 (q, J=6.4, 12 Hz, 2H, H−6a, H−6b), 3.97 (dt, J=4.0, 10.0 Hz, 1H, H−5), 3.77-3.71 (m, 2H, OCH₂O), 3.29 (s, 3H, —OCH₃), 2.84-2.71 (m, 2H, —SCH₂CH₃), 1.26 (t, J=7.2 Hz, 3H, —SCH₂CH₃). ¹³C NMR (150 MHz, CDCl₃) δ 166.0, 165.4, 165.3, 133.5, 133.4, 133.3, 130.0, 129.9, 129.9, 129.4, 129.1, 129.0, 128.6, 128.5, 128.4, 97.0, 83.8, 78.0, 74.5, 70.8, 69.7, 66.7, 55.5, 29.8, 24.3, 15.0.

### Example 69

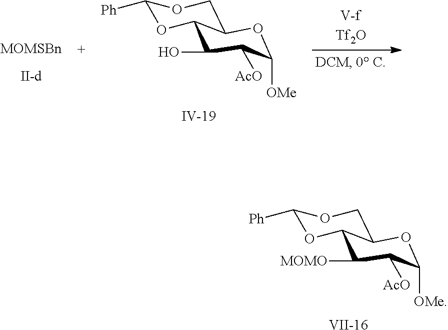

According to the reaction standard operating procedure, the raw materials were as follows: sulfide II-d (10.08 mg, 0.060 mmol, 1.2 equiv), benziodoxole reagent V-f (11.30 mg, 0.015 mmol, 0.3 equiv), acceptor IV-19 (16.2 mg, 0.050 mmol, 1.0 equiv), acid catalyst Tf2O (5 mol %), and after the reaction was complete, the target product VII-16 (13.9 mg, 76%) was obtained. The characterization data of this compound are as follows: ¹H NMR (600 MHz, CDCl₃) δ 7.48-7.42 (m, 2H, Ar—H), 7.37-7.30 (m, 3H, Ar—H), 5.53 (s, 1H, Ph-H), 4.92 (s, 1H, OCH₂O), 4.86-4.80 (m, 2H, OCH₂O, H−2), 4.70 (d, J=6.6 Hz, 1H, H−1), 4.27 (dt, J=1.8, 10.2 Hz, 1H, H−6a), 4.18 (t, J=9.2 Hz, 1H, H−3), 3.84 (td, J=4.8, 10.8 Hz, 1H, H−5), 3.75 (t, J=10.2 Hz, 1H, H−6b), 3.61 (t, J=9.6 Hz, 1H, H−4), 3.38 (s, 3H), 3.33 (s, 3H), 2.12 (s, 3H, OCOCH₃). ¹³C NMR (150 MHz, CDCl₃) δ 170.5, 137.3, 129.2, 128.4, 126.2, 101.7, 97.8, 97.2, 81.6, 73.0, 72.7, 69.1, 62.4, 55.8, 55.4, 21.1.

### Example 70

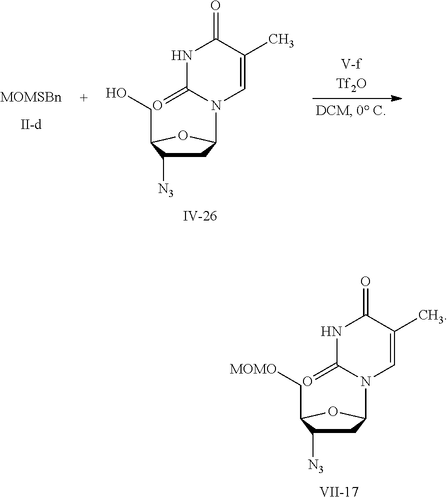

According to the reaction standard operating procedure, the raw materials were as follows: sulfide II-d (10.05 mg, 0.060 mmol, 1.2 equiv), benziodoxole reagent V-f (11.26 mg, 0.015 mmol, 0.3 equiv), acceptor IV-26 (13.3 mg, 0.050 mmol, 1.0 equiv), acid catalyst Tf2O (5 mol %), and after the reaction was complete, the target product VII-17 (11.4 mg, 73%) was obtained. The characterization data of this compound are as follows: ¹H NMR (600 MHz, CDCl₃) δ 8.78 (s, 1H, NH), 7.53 (s, 1H), 6.20 (t, J=6.0 Hz, 1H, H−1), 4.70 (dt, J=4.8, 10.8 Hz, 2H, OCH₂O), 4.27 (dd, J=5.4, 11.4 Hz, 1H, H−3), 4.02 (m, 1H, H−4), 3.85 (dt, J=2.4, 10.8 Hz, 1H, H−5a), 3.73 (dt, J=2.4, 10.8 Hz, 1H, H−5b), 3.38 (s, 3H, OCH₃), 2.45-2.28 (m, 2H, H−2a, H−2b), 1.89 (s, 3H, CH₃). ¹³C NMR (150 MHz, CDCl₃) δ 163.7, 150.3, 135.6, 111.1, 96.9, 84.9, 83.2, 66.9, 60.3, 55.8, 38.1, 12.8.

### Example 71

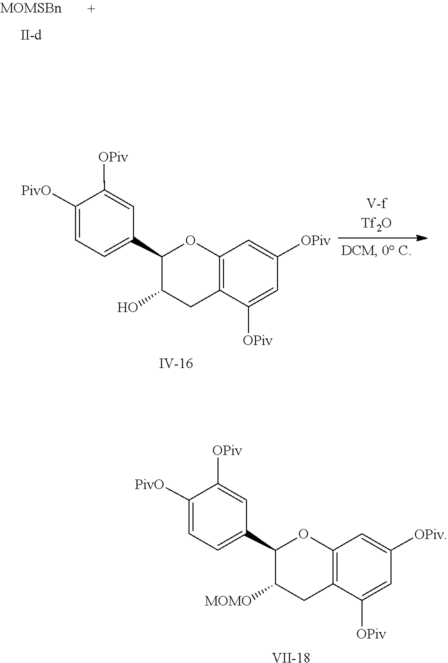

According to the reaction standard operating procedure, the raw materials were as follows: sulfide II-d (10.09 mg, 0.060 mmol, 1.2 equiv), benziodoxole reagent V-f (11.30 mg, 0.015 mmol, 0.3 equiv), acceptor IV-16 (31.3 mg, 0.050 mmol, 1.0 equiv), acid catalyst Tf2O (5 mol %), and after the reaction was complete, the target product VII-18 (28.5 mg, 86%) was obtained. The characterization data of this compound are as follows: ¹H NMR (600 MHz, CDCl₃) δ 7.26-7.24 (m, 1H), 7.14 (t, J=1.8 Hz, 1H), 7.10 (d, J=8.4 Hz, 1H), 6.56 (t, J=2.4 Hz, 1H), 6.47 (t, J=2.4 Hz, 1H), 4.86 (d, J=7.8 Hz, 1H), 4.51 (d, J=7.2 Hz, 1H), 4.29 (d, J=7.2 Hz, 1H), 3.96-3.91 (m, 1H), 3.12 (s, 3H), 2.92 (dd, J=5.4, 16.2 Hz, 1H), 2.64 (dd, J=9.0, 16.2 Hz, 1H), 1.35-1.29 (m, 36H).

### Example 72

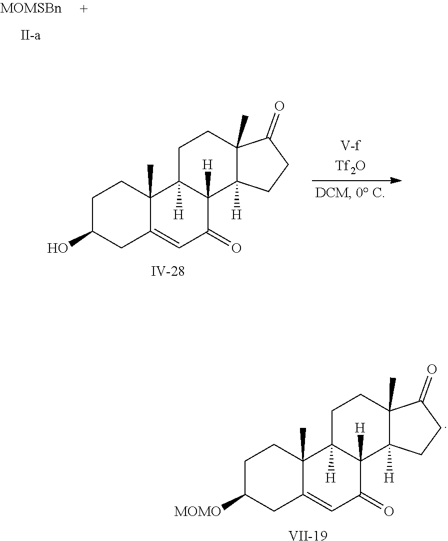

According to the reaction standard operating procedure, the raw materials were as follows: sulfide II-a (13.36 mg, 0.079 mmol, 1.2 equiv), benziodoxole reagent V-f (14.96 mg, 0.020 mmol, 0.3 equiv), acceptor IV-28 (20.0 mg, 0.066 mmol, 1.0 equiv), acid catalyst Tf2O (5 mol %), and after the reaction was complete, the target product VII-19 (16.3 mg, 71%) was obtained. ¹H NMR (400 MHz, CDCl₃) δ 5.72 (d, J=2.0 Hz, 1H), 4.68 (d, J=6.8 Hz, 1H), 4.66 (d, J=6.8 Hz, 1H), 3.58-3.48 (m, 1H), 3.35 (s, 3H), 2.84-2.75 (m, 1H), 2.57 (ddd, J=2.4, 4.8, 14.0 Hz, 1H), 2.49-2.34 (m, 3H), 2.11 (m, 1H), 2.03-1.91 (m, 2H), 1.86-1.79 (m, 1H), 1.79-1.69 (m, 2H), 1.61-1.47 (m, 3H), 1.24-1.15 (m, 6H), 0.87 (s, 3H). ¹³C NMR (150 MHz, CDCl₃) δ 220.5, 201.1, 166.2, 126.1, 95.2, 75.7, 55.5, 50.2, 48.0, 45.9, 45.9, 44.5, 39.7, 38.7, 36.4, 35.8, 30.8, 28.7, 24.3, 20.7, 17.5, 13.9.

### Example 73

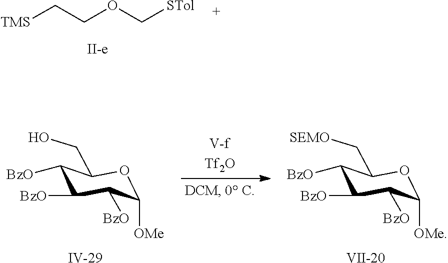

According to the reaction standard operating procedure, the raw materials were as follows: sulfide II-e (18.09 mg, 0.071 mmol, 1.2 equiv), benziodoxole reagent V-f (13.40 mg, 0.018 mmol, 0.3 equiv), acceptor IV-29 (30.0 mg, 0.059 mmol, 1.0 equiv), acid catalyst Tf2O (5 mol %), and after the reaction was complete, the target product VII-20 (37.0 mg, 98%) was obtained. ¹H NMR (400 MHz, CDCl₃) δ 7.86-7.79 (m, 4H, Ar—H), 7.76-7.71 (m, 2H, Ar—H), 7.40-7.34 (m, 2H, Ar—H), 7.32-7.11 (m, 7H, Ar—H), 6.00 (t, J=10.0 Hz, 1H, H−3), 5.50 (t, J=10.0 Hz, 1H, H−4), 5.14 (dd, J=4, 9.6 Hz, 1H, H−2), 5.12 (d, J=4 Hz, 1H, H−1), 4.57 (q, J=6.8, 16 Hz, 2H, H−6a, H−6b), 4.09 (ddd, J=2.8, 4.4, 10.4 Hz, 1H, H−5), 3.66-3.58 (m, 2H), 3.50-3.43 (m, 2H), 3.35 (s, 3H, OMe), 0.75-0.69 (m, 2H), −0.16 (s, 9H, —CH₃×3). ¹³C NMR (150 MHz, CDCl₃) δ 166.0, 165.3, 133.5, 133.2, 130.1, 129.9, 129.8, 129.4, 129.2, 128.5, 128.4, 97.2, 95.4, 72.3, 70.8, 69.4, 68.9, 66.1, 65.4, 55.7, 18.1, −1.3.

### Example 74

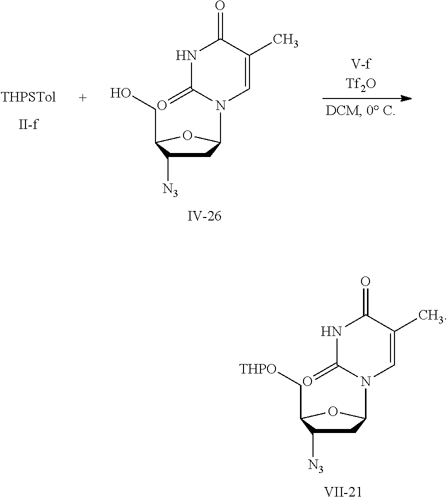

According to the reaction standard operating procedure, the raw materials were as follows: sulfide II-f (18.71 mg, 0.090 mmol, 1.2 equiv), benziodoxole reagent V-f (16.93 mg, 0.022 mmol, 0.3 equiv), acceptor IV-26 (20.0 mg, 0.075 mmol, 1.0 equiv), acid catalyst Tf2O (5 mol %), and after the reaction was complete, the target product VII-21 (25.6 mg, 97%) was obtained. The characterization data of this compound are as follows: ¹H NMR (400 MHz, CDCl₃) δ 8.91 (s, 1H), 7.55 (dd, J=1.6, 9.2 Hz, 1H), 6.22 (m, 1H), 4.66-4.61 (m, 0.55H), 4.60-4.56 (m, 0.45H), 4.38-4.31 (m, 0.55H), 4.28-4.22 (m, 0.45H), 4.09 (dd, J=2.8, 11.2 Hz, 0.45H), 4.07-4.02 (m, 1H), 3.95 (dd, J=2.4, 11.6 Hz, 0.55H), 3.92-3.86 (m, 0.45H), 3.85-3.79 (m, 0.55H), 3.76 (m, 0.55H), 3.61-3.50 (m, 1.45H), 2.45-2.37 (m, 1H), 2.33-2.21 (m, 1H), 1.91 (d, J=1.2 Hz, 1.35H), 1.88 (d, J=1.2 Hz, 1.65H), 1.84-1.73 (m, 2H), 1.61-1.49 (m, 4H). ¹³C NMR (150 MHz, CDCl₃) δ 164.0, 164.0, 150.4, 135.7, 135.5, 111.1, 111.0, 100.2, 99.4, 85.1, 84.9, 83.6, 83.3, 67.2, 66.7, 63.6, 63.2, 61.0, 60.7, 38.3, 38.1, 30.8, 30.8, 25.3, 25.3, 20.3, 20.0, 12.7, 12.5.

### Example 75

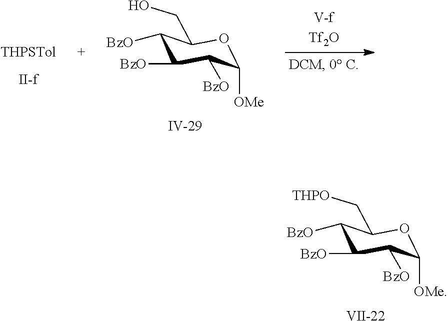

According to the reaction standard operating procedure, the raw materials were as follows: sulfide II-f (14.81 mg, 0.071 mmol, 1.2 equiv), benziodoxole reagent V-f (13.40 mg, 0.018 mmol, 0.3 equiv), acceptor IV-29 (30.0 mg, 0.059 mmol, 1.0 equiv), acid catalyst Tf2O (5 mol %), and after the reaction was complete, the target product VII-22 (34.3 mg, 98%) was obtained. The characterization data of this compound are as follows: ¹H NMR (600 MHz, CDCl₃) δ 8.00-7.97 (m, 2H), 7.96-7.92 (m, 2H), 7.90-7.85 (m, 2H), 7.53-7.48 (m, 2H), 7.44-7.40 (m, 1H), 7.39-7.35 (m, 4H), 7.31-7.26 (m, 2H), 6.17-6.11 (m, 1H), 5.66 (t, J=10.2 Hz, 0.57H), 5.60 (t, J=10.2 Hz, 0.43H), 5.28 (dt, J=3.6, 10.2 Hz, 1H), 5.25 (d, J=3.6 Hz, 1H), 4.66 (t, J=3.6 Hz, 0.57H), 4.56 (t, J=3.6 Hz, 0.43H), 4.28-4.23 (m, 1H), 3.98-3.94 (m, 1H), 3.86-3.82 (m, 0.43H), 3.71-3.66 (m, 0.57H), 3.64-3.59 (m, 1H), 3.48 (s, 3H), 3.47-3.43 (m, 0.43H), 3.39-3.34 (m, 0.57H), 1.85-1.42 (m, 6H) ¹³C NMR (150 MHz, CDCl₃) δ 166.0, 165.4, 165.4, 133.5, 133.4, 133.2, 130.1, 129.9, 129.9, 129.8, 129.8, 129.5, 129.4, 129.3, 129.3, 128.5, 128.5, 128.4, 99.8, 98.4, 97.1, 97.0, 72.3, 72.3, 70.9, 70.8, 69.8, 69.6, 69.2, 68.6, 66.3, 65.7, 62.4, 61.6, 55.6, 55.6, 30.5, 30.4, 25.5, 25.5, 19.5, 18.9.

### Example 76

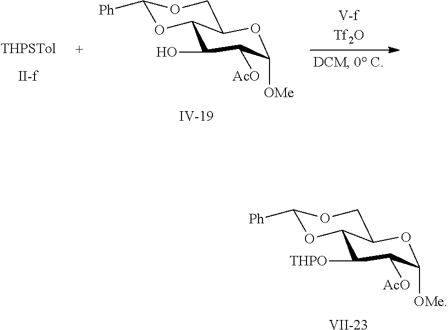

According to the reaction standard operating procedure, the raw materials were as follows: sulfide II-f (15.42 mg, 0.074 mmol, 1.2 equiv), benziodoxole reagent V-f (13.95 mg, 0.018 mmol, 0.3 equiv), acceptor IV-19 (20.0 mg, 0.061 mmol, 1.0 equiv), acid catalyst Tf2O (5 mol %), and after the reaction was complete, the target product VII-23 (23.9 mg, 95%) was obtained. The characterization data of this compound are as follows: ¹H NMR (400 MHz, CDCl₃) δ 7.42-7.38 (m, 0.64H), 7.36-7.30 (m, 1.36H), 7.29-7.19 (m, 3H), 5.48 (s, 0.36H), 5.43 (s, 0.64H), 4.95 (t, J=3.2 Hz, 0.64H), 4.84-4.78 (m, 2H), 4.76 (dd, J=4.0, 9.6 Hz, 0.36H), 4.21-4.10 (m, 2H), 3.93 (td, J=2.8, 11.2 Hz, 0.36H), 3.83-3.71 (m, 1.64H), 3.68-3.61 (m, 1H), 3.57-3.50 (m, 1H), 3.44-3.38 (m, 0.64H), 3.29 (s, 1.92H), 3.28 (s, 1.08H), 3.27-3.21 (m, 0.36H), 2.04 (s, 1.92H), 2.00 (s, 1.08H), 1.66-1.30 (m, 6H). ¹³C NMR (150 MHz, CDCl₃) δ 170.8, 170.6, 170.4, 137.5, 137.4, 137.1, 129.5, 129.1, 129.0, 128.5, 128.4, 128.2, 126.4, 126.2, 126.1, 102.2, 101.5, 101.4, 98.5, 98.5, 98.0, 97.8, 97.7, 94.8, 94.5, 82.7, 81.6, 80.3, 74.5, 72.8, 72.1, 72.1, 69.1, 69.0, 68.8, 64.0, 63.1, 62.8, 62.3, 62.1, 61.4, 61.2, 55.5, 55.4, 55.3, 32.1, 30.8, 30.5, 30.5, 29.8, 25.6, 25.6, 25.5, 25.5, 25.4, 21.2, 21.1, 21.1, 20.4, 19.9, 18.7, 18.5.

### Example 77

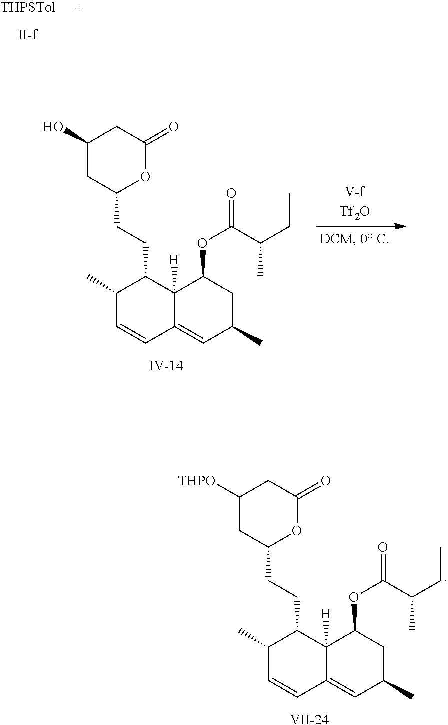

According to the reaction standard operating procedure, the raw materials were as follows: sulfide II-f (12.36 mg, 0.059 mmol, 1.2 equiv), benziodoxole reagent V-f (11.19 mg, 0.015 mmol, 0.3 equiv), acceptor IV-14 (20.0 mg, 0.049 mmol, 1.0 equiv), acid catalyst Tf2O (5 mol %), and after the reaction was complete, the target product VII-24 (22.9 mg, 95%) was obtained. The characterization data of this compound are as follows: ¹H NMR (400 MHz, CDCl₃) δ 5.97 (d, J=9.6 Hz, 1H), 5.76 (dd, J=6.0, 9.6 Hz 1H), 5.50 (t, J=3.6 Hz, 1H), 5.37-5.31 (m, 1H), 4.66 (dt, J=3.2, 10.8 Hz, 1H), 4.62-4.42 (m, 1H), 4.26-4.18 (m, 1H), 3.84-3.75 (m, 1H), 3.54-3.45 (m, 1H), 2.82-2.67 (m, 1H), 2.67-2.52 (m, 1H), 2.44-2.20 (m, 4H), 1.56-1.21 (m, 10H), 1.08 (d, J=6.8 Hz, 1H), 1.05 (d, J=7.6 Hz, 1H), 0.89-0.82 (m, 6H). ¹³C NMR (150 MHz, CDCl₃) δ 176.72, 176.69, 170.46, 170.21, 133.25, 133.22, 131.82, 131.77, 129.78, 128.45, 97.45, 97.21, 76.71, 67.99, 67.12, 66.89, 63.10, 62.45, 41.53, 37.39, 37.34, 36.85, 36.81, 35.67, 35.16, 33.53, 33.33, 32.72, 32.67, 30.95, 30.91, 30.84, 27.60, 26.96, 25.43, 25.42, 24.44, 24.41, 22.93, 19.75, 19.27, 16.40, 16.35, 14.03, 11.79.

### Example 78

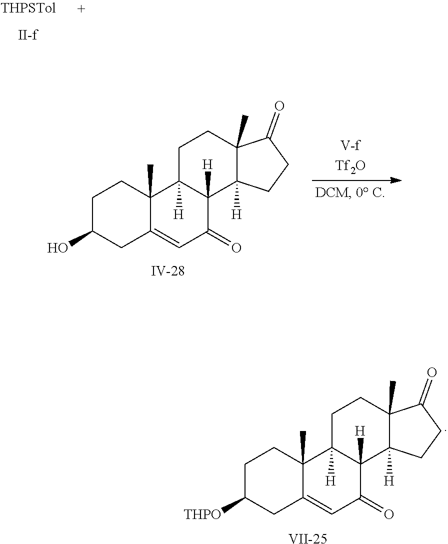

According to the reaction standard operating procedure, the raw materials were as follows: sulfide II-f (16.54 mg, 0.079 mmol, 1.2 equiv), benziodoxole reagent V-f (14.96 mg, 0.020 mmol, 0.3 equiv), acceptor IV-28 (20.0 mg, 0.066 mmol, 1.0 equiv), acid catalyst Tf2O (5 mol %), and after the reaction was complete, the target product VII-25 (25.0 mg, 98%) was obtained. The characterization data of this compound are as follows: ¹H NMR (400 MHz, CDCl₃) δ 5.71 (dd, J=2.0, 7.6 Hz, 1H), 4.72-4.66 (m, 1H), 3.87 (ddd, J=3.6, 6.8, 14.4 Hz, 1H), 3.62 (m, 1H), 3.51-3.43 (m, 1H), 2.79 (m, 1H), 2.64-1.90 (m, 8H), 1.86-1.46 (m, 11H), 1.26-1.11 (m, 6H), 0.86 (s, 3H). ¹³C NMR (150 MHz, CDCl₃) δ 220.5, 220.5, 201.2, 166.8, 166.5, 126.0, 126.0, 97.6, 97.3, 74.9, 74.7, 63.1, 63.1, 50.2, 48.0, 45.9, 44.5, 40.3, 39.0, 38.8, 38.8, 36.6, 36.3, 35.8, 31.3, 31.2, 30.9, 29.5, 27.8, 25.5, 24.3, 20.7, 20.7, 20.0, 20.0, 17.5, 13.9.

### Example 79

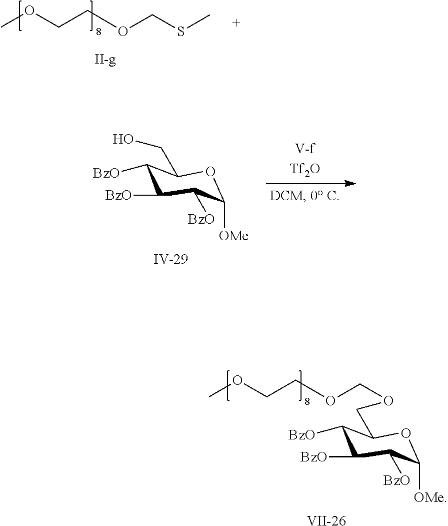

According to the reaction standard operating procedure, the raw materials were as follows: sulfide II-g (10.54 mg, 0.024 mmol, 1.2 equiv), benziodoxole reagent V-f (4.47 mg, 0.059 mmol, 0.3 equiv), acceptor IV-29 (10.0 mg, 0.020 mmol, 1.0 equiv), acid catalyst Tf2O (5 mol %), and after the reaction was complete, the target product VII-26 (17.1 mg, 96%) was obtained. The characterization data of this compound are as follows: ¹H NMR (600 MHz, CDCl₃) δ 7.97-7.93 (m, 2H, Ar—H), 7.93-7.89 (m, 2H, Ar—H), 7.86-7.82 (m, 2H, Ar—H), 7.52-7.46 (m, 2H, Ar—H), 7.42-7.38 (m, 1H, Ar—H), 7.38-7.33 (m, 4H, Ar—H), 7.29-7.25 (m, 2H, Ar—H), 6.11 (t, J=9.6 Hz, 1H, H−3), 5.63 (t, J=9.6 Hz, 1H, H−4), 5.25 (dd, J=3.6, 9.6 Hz, 1H, H−2), 5.22 (d, J=3.6 Hz, 1H, H−1), 4.74 (d, J=6.6 Hz, 1H, OCH₂O), 4.71 (d, J=6.6 Hz, 1H, OCH₂O), 4.19 (dt, J=3.0, 10.2 Hz, 1H, H−5), 3.77-3.70 (m, 2H, H−6a, H−6b), 3.68-3.48 (m, 32H), 3.50 (s, 3H), 3.35 (s, 3H). 13C NMR (150 MHz, CDCl₃) δ 166.0, 165.4, 133.5, 133.5, 133.2, 130.1, 129.9, 129.8, 129.4, 129.2, 129.2, 128.6, 128.5, 128.4, 97.2, 95.9, 72.2, 72.1, 70.8, 70.7, 70.7, 70.7, 70.6, 70.5, 69.4, 68.8, 67.1, 66.0, 59.2, 55.8.

### Example 80

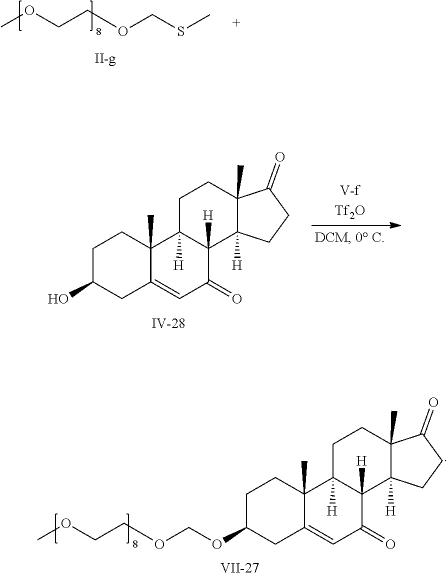

According to the reaction standard operating procedure, the raw materials were as follows: sulfide II-g (17.65 mg, 0.040 mmol, 1.2 equiv), benziodoxole reagent V-f (7.49 mg, 0.099 mmol, 0.3 equiv), acceptor V-28 (10.0 mg, 0.033 mmol, 1.0 equiv), acid catalyst Tf2O (5 mol %), and after the reaction was complete, the target product VII-27 (18.9 mg, 82%) was obtained through silica gel column chromatography separation and purification. The characterization data of this compound are as follows: ¹H NMR (400 MHz, CDCl₃) δ 5.71 (d, J=1.6 Hz, 1H), 4.82-4.71 (m, 2H), 3.72-3.65 (m, 2H), 3.65-3.49 (m, 32H), 3.35 (s, 3H), 2.84-2.74 (m, 1H), 2.57 (ddd, J=2.4, 4.8, 14.0 Hz, 1H), 2.49-2.32 (m, 3H), 2.17-2.05 (m, 1H), 2.02-1.90 (m, 2H), 1.87-1.68 (m, 6H), 1.661.51 (m, 4H), 1.19 (s, 3H), 0.87 (s, 3H). ¹³C NMR (150 MHz, CDCl₃) δ 220.5, 201.1, 166.3, 126.1, 94.1, 75.6, 72.1, 70.8, 70.8, 70.7, 70.7, 70.6, 67.2, 59.2, 50.2, 48.0, 45.9, 44.5, 39.6, 38.7, 36.4, 35.8, 30.9, 28.6, 24.3, 20.7, 17.5, 13.9.

### Example 81

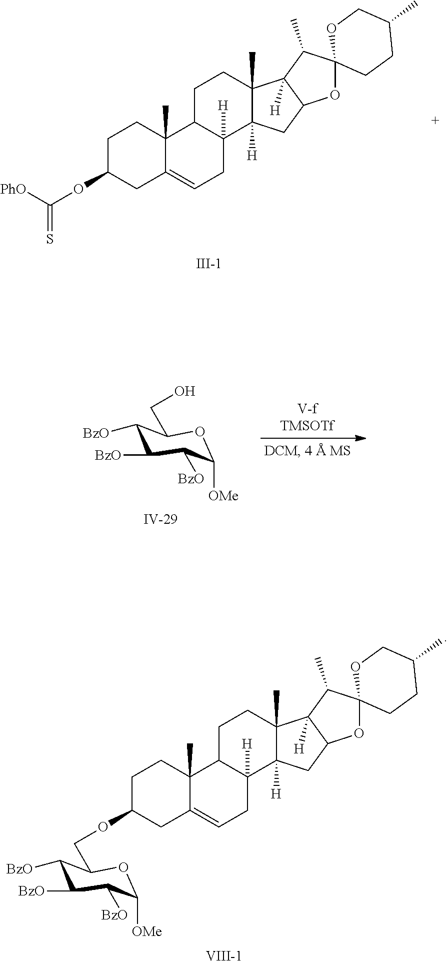

According to the reaction standard operating procedure, diosgenin thiocarbonate III-1 (124.8 mg, 0.045 mmol, 1.5 equiv) and acceptor IV-29 (15.2 mg, 0.03 mmol, 1.0 equiv), benziodoxole reagent V-f (9.0 mg, 0.012 mmol, 0.4 equiv), and pre-activated molecular sieves were added to the reaction flask. Under argon atmosphere, anhydrous DCM (c=0.1 M, calculated based on the reactant with 1.0 equivalent) was added. The reaction system was pre-cooled to 0° C., TMSOTf (2.2 μL, 0.012 mmol, 0.4 equiv) was added, and the reaction was carried out at 0° C. for 7 hours. After purification by silica gel column chromatography, the target product VIII-1 (25.0 mg, 92%) was obtained. The characterization data of this compound are as follows: 1H NMR (600 MHz, CDCl₃) δ 8.00-7.89 (m, 4H), 7.86-7.82 (m, 2H), 7.52-7.47 (m, 2H), 7.41-7.34 (m, 5H), 7.26 (t, J=7.8 Hz, 2H), 6.11 (t, J=9.6 Hz, 1H), 5.63 (t, J=9.6 Hz, 1H), 5.24 (dt, J=10.2, 3.6 Hz, 3H), 4.39-4.35 (m, 1H), 4.17-4.14 (m, 1H), 3.73 (dd, J=10.8, 3.0 Hz, 1H), 3.59 (dd, J=10.8, 4.8 Hz, 1H), 3.48-3.39 (m, 4H), 3.35 (t, J=10.8 Hz, 1H), 3.10-3.02 (m, 1H), 2.27-2.24 (m, 1H), 2.19-2.11 (m, 1H), 2.00-1.90 (m, 2H), 1.89-1.35 (m, 18H), 1.28-1.23 (m, 1H), 1.15-1.10 (m, 1H), 1.08-1.03 (m, 1H), 1.00-0.85 (m, 8H), 0.79-0.72 (m, 6H).

### Example 82

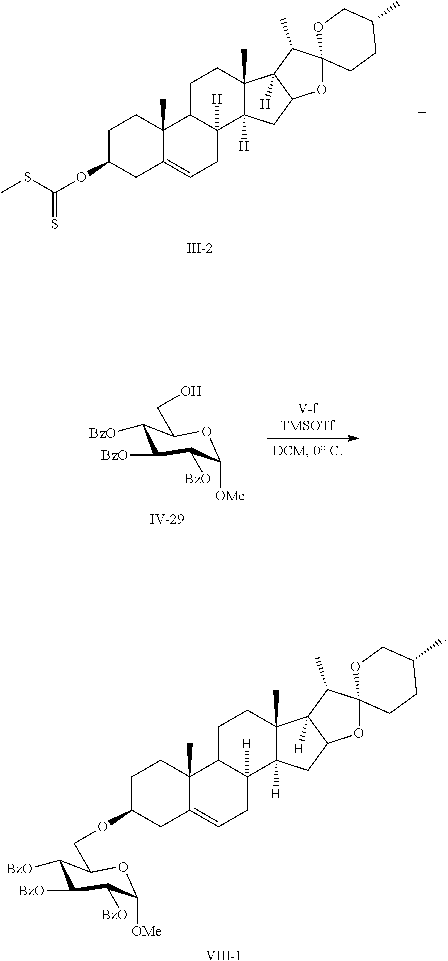

According to the reaction standard operating procedure, diosgenin xanthate III-2 (22.7 mg, 0.045 mmol, 1.5 equiv) and acceptor IV-29 (15.2 mg, 0.03 mmol, 1.0 equiv), benziodoxole reagent V-f (9.0 mg, 0.012 mmol, 0.4 equiv), and pre-activated molecular sieves were added to the reaction flask. Under argon atmosphere, anhydrous DCM (c=0.1 M, calculated based on the reactant with 1.0 equivalent) was added. The reaction system was pre-cooled to 0° C., TMSOTf (2.2 μL, 0.012 mmol, 0.4 equiv) was added, and the reaction was carried out at 0° C. for 12 hours. After purification by silica gel column chromatography, the target product (23.3 mg, 86%) was obtained. The characterization data of this compound are as follows: ¹H NMR (600 MHz, CDCl₃) δ 8.00-7.89 (m, 4H), 7.86-7.82 (m, 2H), 7.52-7.47 (m, 2H), 7.41-7.34 (m, 5H), 7.26 (t, J=7.8 Hz, 2H), 6.11 (t, J=9.6 Hz, 1H), 5.63 (t, J=9.6 Hz, 1H), 5.24 (dt, J=10.2, 3.6 Hz, 3H), 4.39-4.35 (m, 1H), 4.17-4.14 (m, 1H), 3.73 (dd, J=10.8, 3.0 Hz, 1H), 3.59 (dd, J=10.8, 4.8 Hz, 1H), 3.48-3.39 (m, 4H), 3.35 (t, J=10.8 Hz, 1H), 3.10-3.02 (m, 1H), 2.27-2.24 (m, 1H), 2.19-2.11 (m, 1H), 2.00-1.90 (m, 2H), 1.89-1.35 (m, 18H), 1.28-1.23 (m, 1H), 1.15-1.10 (m, 1H), 1.08-1.03 (m, 1H), 1.00-0.85 (m, 8H), 0.79-0.72 (m, 6H).

### Example 83

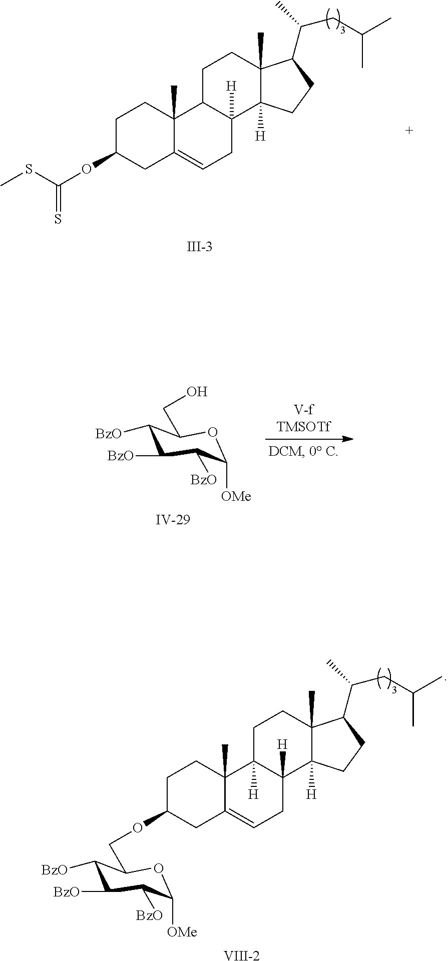

According to the reaction standard operating procedure, cholesterol xanthate III-3 (21.4 mg, 0.045 mmol, 1.5 equiv) and acceptor IV-29 (15.2 mg, 0.03 mmol, 1.0 equiv), benziodoxole reagent V-f (9.0 mg, 0.012 mmol, 0.4 equiv), and pre-activated molecular sieves were added to the reaction flask. Under argon atmosphere, anhydrous DCM (c=0.1 M, calculated based on the reactant with 1.0 equivalent) was added. The reaction system was pre-cooled to 0° C., TMSOTf (2.2 μL, 0.012 mmol, 0.4 equiv) was added, and the reaction was carried out at 0° C. for 12 hours. After purification by silica gel column chromatography, the target product VIII-2 (27.3 mg, 84%) was obtained. The characterization data of this compound are as follows: ¹H NMR (600 MHz, CDCl₃) δ 7.95 (m, 4H), 7.85 (d, J=7.8 Hz, 2H), 7.49 (m, 2H), 7.43-7.33 (m, 5H), 7.26 (t, J=7.8 Hz, 2H), 6.11 (t, J=9.6 Hz, 1H), 5.63 (t, J=9.6 Hz, 1H), 5.28-5.21 (m, 3H), 4.20-4.14 (m, 1H), 3.73 (dd, J=10.8, 2.4 Hz, 1H), 3.60 (dd, J=10.8, 4.8 Hz, 1H), 3.46 (s, 3H), 3.13-3.03 (m, 1H), 2.25 (dd, J=13.2, 2.4 Hz, 1H), 2.15 (dd, J=18.0, 6.6 Hz, 1H), 1.99-1.76 (m, 5H), 1.58-0.79 (m, 36H), 0.64 (s, 3H).

### Example 84

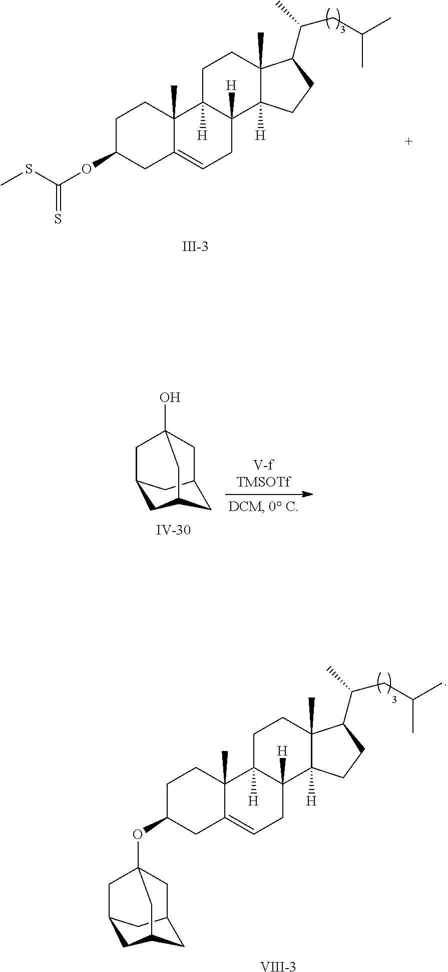

According to the reaction standard operating procedure, cholesterol xanthate III-3 (21.4 mg, 0.045 mmol, 1.5 equiv) and acceptor IV-30 (4.6 mg, 0.03 mmol, 1.0 equiv), benziodoxole reagent V-f (9.0 mg, 0.012 mmol, 0.4 equiv), and pre-activated molecular sieves were added to the reaction flask. Under argon atmosphere, anhydrous DCM (c=0.1 M, calculated based on the reactant with 1.0 equivalent) was added. The reaction system was pre-cooled to 0° C., TMSOTf (2.2 μL, 0.012 mmol, 0.4 equiv) was added, and the reaction was continued at 0° C. for 2 hours. After purification by silica gel column chromatography, the target product VIII-3 (14.3 mg, 92%) was obtained. The characterization data of this compound are as follows: ¹H NMR (600 MHz, CDCl₃) δ 5.30-5.25 (m, 1H), 3.47-3.41 (m, 1H), 2.26 (m, 1H), 2.13-2.05 (m, 4H), 2.02-1.92 (m, 2H), 1.81-0.85 (m, 50H), 0.65 (s, 3H).

### Example 85

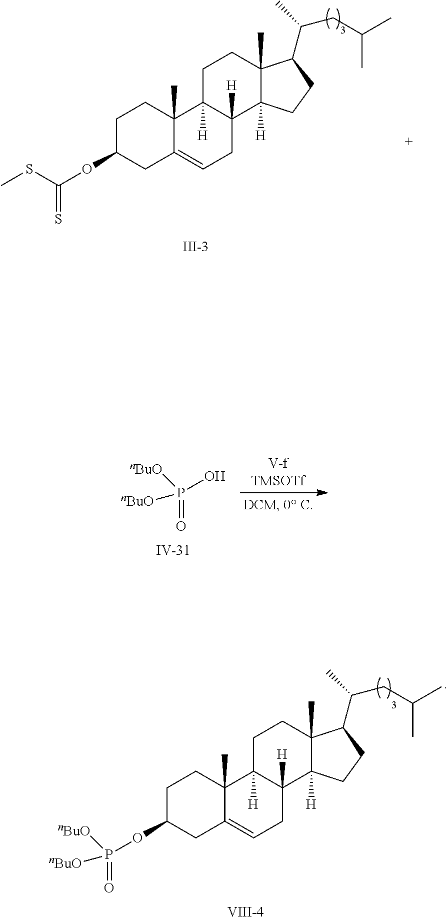

According to the reaction standard operating procedure, cholesterol xanthate III-3 (21.4 mg, 0.045 mmol, 1.5 equiv) and acceptor IV-31 (6.3 mg, 0.03 mmol, 1.0 equiv), benziodoxole reagent V-f (9.0 mg, 0.012 mmol, 0.4 equiv), and pre-activated molecular sieves were added to the reaction flask. Under argon atmosphere, anhydrous DCM (c=0.1 M, calculated based on the reactant with 1.0 equivalent) was added. The reaction system was pre-cooled to 0° C., TMSOTf (2.2 μL, 0.012 mmol, 0.4 equiv) was added, and the reaction was continued at 0° C. for 4 hours. After purification by silica gel column chromatography, the target product VIII-4 (16.9 mg, 97%) was obtained. The characterization data of this compound are as follows: ¹H NMR (600 MHz, CDCl₃) δ 5.35 (d, J=4.8 Hz, 1H), 4.15 (dt, J=10.8, 4.8 Hz, 1H), 4.00 (m, 4H), 2.43-2.36 (m, 2H), 2.01-1.92 (m, 3H), 1.86-1.77 (m, 2H), 1.67-0.83 (m, 48H), 0.65 (s, 3H).

### Example 86

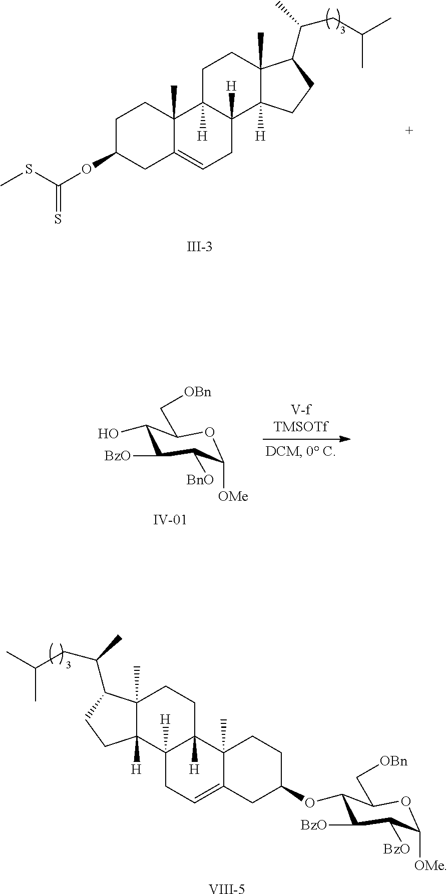

According to the reaction standard operating procedure, cholesterol xanthate III-3 (21.4 mg, 0.045 mmol, 1.5 equiv) and acceptor IV-01 (13.9 mg, 0.03 mmol, 1.0 equiv), benziodoxole reagent V-f (9.0 mg, 0.012 mmol, 0.4 equiv), and pre-activated molecular sieves were added to the reaction flask. Under argon atmosphere, anhydrous DCM (c=0.1 M, calculated based on the reactant with 1.0 equivalent) was added. The reaction system was pre-cooled to 0° C., TMSOTf (2.2 μL, 0.012 mmol, 0.4 equiv) was added, and the reaction was carried out at 0° C. for 2 hours. After purification by silica gel column chromatography, the target product VIII-5 (23.6 mg, 94%) was obtained. The characterization data of this compound are as follows: ¹H NMR (400 MHz, CDCl₃) δ 7.31-7.19 (m, 15H), 5.16 (d, J=4.8 Hz, 1H), 4.87 (d, J=10.4 Hz, 1H), 4.75-4.68 (m, 2H), 4.58-4.49 (m, 3H), 4.43 (d, J=12.0 Hz, 1H), 3.79 (t, J=9.2 Hz, 1H), 3.64-3.56 (m, 3H), 3.52-3.41 (m, 3H), 3.31 (s, 3H), 2.38-2.30 (m, 1H), 2.14-2.08 (m, 1H), 1.95-1.86 (m, 2H), 1.79-1.65 (m, 3H), 1.47-0.79 (m, 34H), 0.61 (s, 3H).

### Example 87

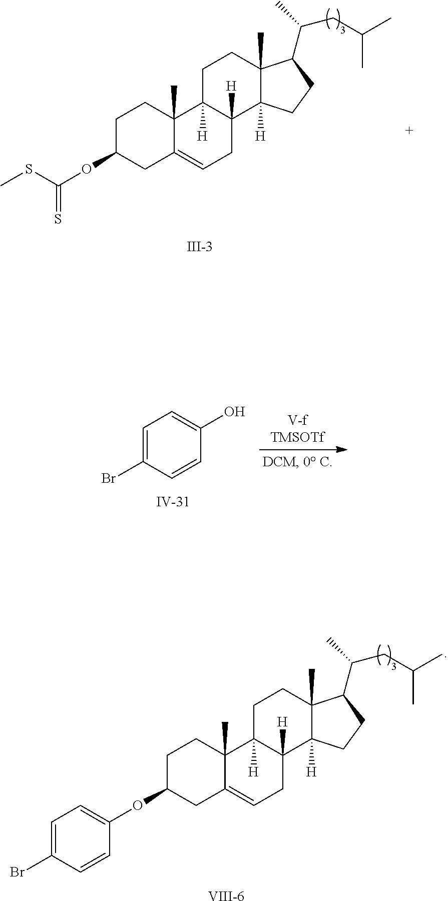

According to the reaction standard operating procedure, cholesterol xanthate III-3 (14.3 mg, 0.03 mmol, 1.0 equiv) and acceptor IV-31 (7.8 mg, 0.045 mmol, 1.5 equiv), benziodoxole reagent V-f (9.0 mg, 0.012 mmol, 0.4 equiv), and pre-activated molecular sieves were added to the reaction flask. Under argon atmosphere, anhydrous DCM (c=0.1 M, calculated based on the reactant with 1.0 equivalent) was added. The reaction system was pre-cooled to 0° C., TMSOTf (2.2 μL, 0.012 mmol, 0.4 equiv) was added, and the reaction was carried out at 0° C. for 6 hours. After purification by silica gel column chromatography, the target product VIII-6 (12.0 mg, 74%) was obtained. The characterization data of this compound are as follows: ¹H NMR (400 MHz, CDCl₃) δ 7.37-7.25 (m, 2H), 6.75 (d, J=8.8 Hz, 2H), 5.37 (d, J=5.2 Hz, 1H), 4.14-3.95 (m, 1H), 2.40 (m, 2H), 2.06-1.93 (m, 3H), 1.89 (m, 1H), 1.85-1.75 (m, 1H), 1.71-0.84 (m, 35H), 0.66 (s, 3H).

### Example 88

The reaction formula is as follows:

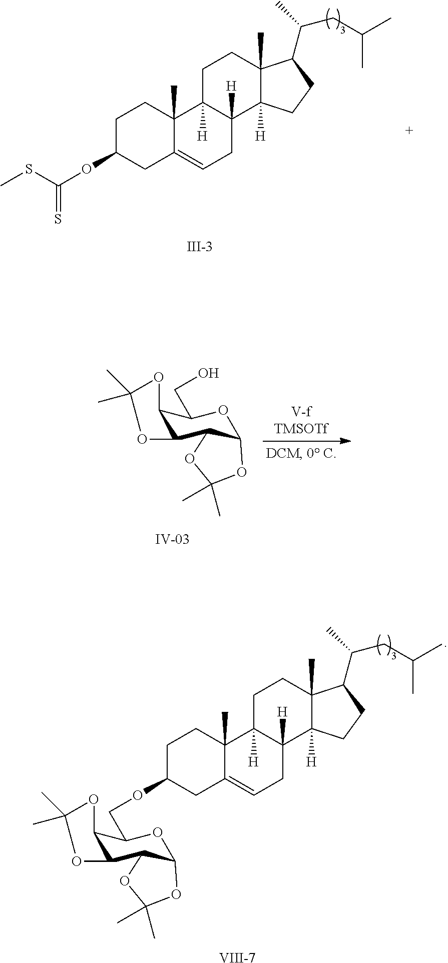

According to the reaction standard operating procedure, cholesterol xanthate III-3 (21.4 mg, 0.045 mmol, 1.5 equiv) and acceptor IV-03 (7.8 mg, 0.03 mmol, 1.0 equiv), benziodoxole reagent V-f (9.0 mg, 0.012 mmol, 0.4 equiv), and pre-activated molecular sieves were added to the reaction flask. Under argon atmosphere, anhydrous DCM (c=0.1 M, calculated based on the reactant with 1.0 equivalent) was added. The reaction system was pre-cooled to 0° C., TMSOTf (2.2 μL, 0.012 mmol, 0.4 equiv) was added, and the reaction was carried out at 0° C. for 3 hours. After purification by silica gel column chromatography, the target product VIII-7 (17.9 mg, 95%) was obtained. The characterization data of this compound are as follows: ¹H NMR (600 MHz, CDCl₃) δ 5.51 (d, J=4.8 Hz, 1H), 5.41-5.27 (m, 1H), 4.57 (dd, J=7.8, 2.4 Hz, 1H), 4.28-4.24 (m, 2H), 3.91-3.89 (m, 1H), 3.66-3.58 (m, 2H), 3.21-3.16 (m, 1H), 2.37-2.33 (m, 1H), 2.22-2.15 (m, 1H), 2.01-1.76 (m, 5H), 1.63-0.83 (m, 48H), 0.65 (s, 3H).

### Example 89

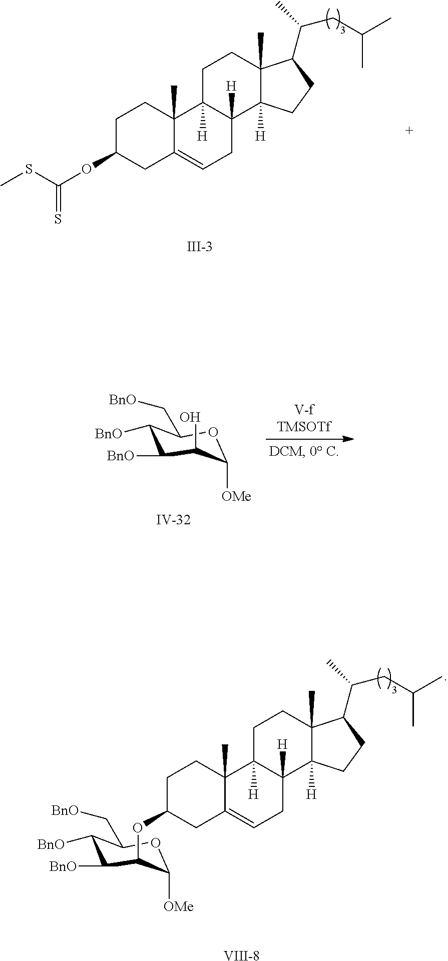

According to the reaction standard operating procedure, cholesterol xanthate III-3 (14.3 mg, 0.045 mmol, 1.5 equiv) and acceptor IV-32 (13.9 mg, 0.03 mmol, 1.0 equiv), benziodoxole reagent V-f (9.0 mg, 0.012 mmol, 0.4 equiv), and pre-activated molecular sieves were added to the reaction flask. Under argon atmosphere, anhydrous DCM (c=0.1 M, calculated based on the reactant with 1.0 equivalent) was added. The reaction system was pre-cooled to 0° C., TMSOTf (2.2 μL, 0.012 mmol, 0.4 equiv) was added, and the reaction was carried out at 0° C. for 6 hours. After purification by silica gel column chromatography, the target product VIII-8 (20.0 mg, 80%) was obtained. The characterization data of this compound are as follows: ¹H NMR (600 MHz, CDCl₃) δ 7.35-7.22 (m, 13H), 7.16-7.10 (m, 2H), 5.36-5.28 (m, 1H), 4.83 (d, J=10.8 Hz, 1H), 4.70 (dd, J=10.2, 6.6 Hz, 2H), 4.66-4.62 (m, 2H), 4.53 (d, J=12.0 Hz, 1H), 4.46 (d, J=10.8 Hz, 1H), 3.90-3.68 (m, 6H), 3.37-3.28 (m, 4H), 2.43-2.40 (m, 1H), 2.35-2.26 (m, 1H), 2.04-1.92 (m, 2H), 1.81 (dd, J=9.8, 4.2 Hz, 3H), 1.66-0.80 (m, 37H), 0.66 (s, 3H).

### Example 90

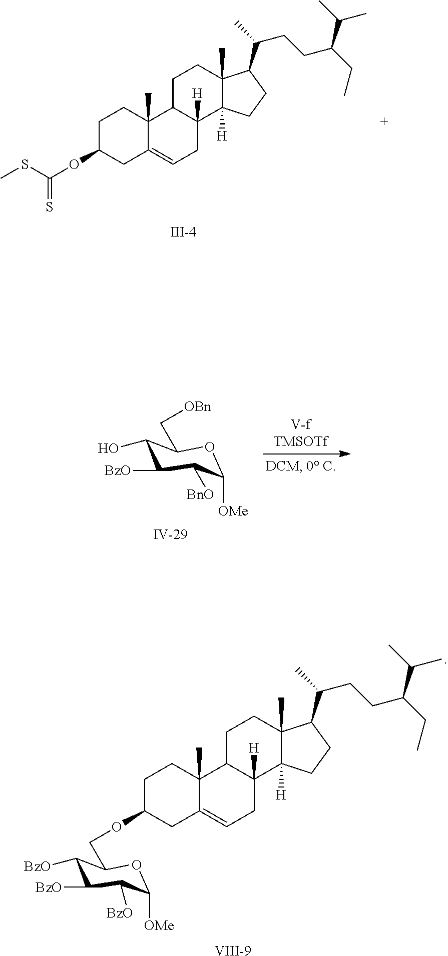

According to the reaction standard operating procedure, 1.0 equiv), benziodoxole reagent V-f (9.0 mg, 0.012 mmol, 0.4 equiv), and pre-activated molecular sieves were added to the reaction flask. Under argon atmosphere, anhydrous DCM (c=0.1 M, calculated based on the reactant with 1.0 equivalent) was added. The reaction system was pre-cooled to 0° C., TMSOTf (2.2 μL, 0.012 mmol, 0.4 equiv) was added, and the reaction was carried out at 0° C. for 12 hours. After purification by silica gel column chromatography, the target product VIII-9 (24.1 mg, 89%) was obtained. The characterization data of this compound are as follows: ¹H NMR (600 MHz, CDCl₃) δ 8.00-7.90 (m, 4H), 7.87-7.83 (m, 2H), 7.52-7.47 (m, 2H), 7.42-7.38 (m, 1H), 7.38-7.33 (m, 4H), 7.29-7.25 (m, 2H), 6.11 (t, J=9.6 Hz, 1H), 5.63 (t, J=9.6 Hz, 1H), 5.26-5.23 (m, 3H), 4.17 (ddd, J=10.8, 4.8, 3.0 Hz, 1H), 3.73 (dd, J=10.8, 3.0 Hz, 1H), 3.60 (dd, J=10.8, 4.8 Hz, 1H), 3.46 (s, 3H), 3.11-3.06 (m, 1H), 2.26-2.23 (m, 1H), 2.20-2.11 (m, 1H), 2.01-1.76 (m, 5H), 1.66-1.61 (m, 1H), 1.53-0.79 (m, 36H), 0.64 (s, 3H). ¹³C NMR (150 MHz, CDCl₃) δ 166.1, 165.4, 141.0, 133.5, 133.5, 133.2, 130.2, 130.0, 129.9, 129.6, 129.5, 129.4, 128.6, 128.5, 121.8, 97.2, 80.5, 72.4, 70.9, 69.9, 69.2, 67.1, 57.0, 56.3, 55.7, 50.3, 46.0, 42.5, 40.0, 39.1, 37.4, 37.0, 36.4, 34.2, 32.1, 32.1, 29.4, 28.5, 28.3, 26.3, 24.5, 23.3, 21.3, 20.0, 19.6, 19.2, 19.0, 12.2, 12.1. HRMS (ESI-TOF) m/z: [M+Na]⁺ calc, for C₅₇H₇₄O₉Na 925.5225, found: 925.5219.

### Example 91

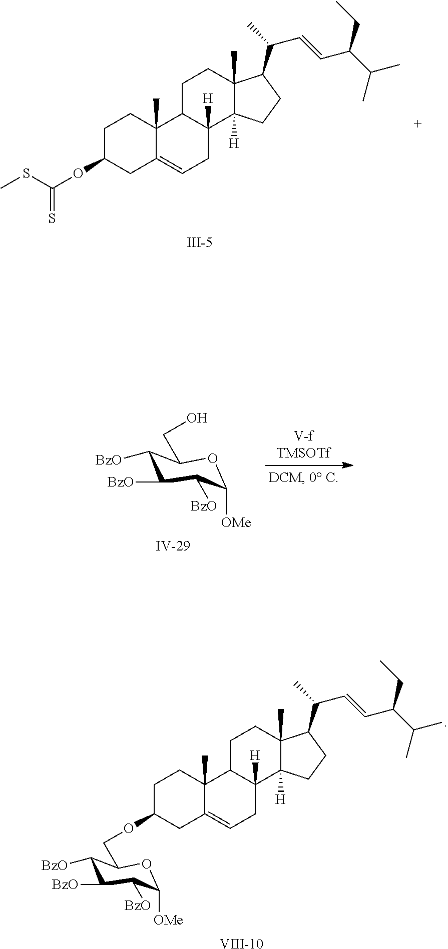

According to the reaction standard operating procedure, stigmasterol xanthate III-4 (22.7 mg, 0.045 mmol, 1.5 equiv) and acceptor IV-29 (15.2 mg, 0.03 mmol, 1.0 equiv), benziodoxole reagent V-f (9.0 mg, 0.012 mmol, 0.4 equiv), and pre-activated molecular sieves were added to the reaction flask. Under argon atmosphere, anhydrous DCM (c=0.1 M, calculated based on the reactant with 1.0 equivalent) was added. The reaction system was pre-cooled to 0° C., TMSOTf (2.2 μL, 0.012 mmol, 0.4 equiv) was added, and the reaction was carried out at 0° C. for 12 hours. After purification by silica gel column chromatography, the target product VIII-10 (21.8 mg, 80%) was obtained. The characterization data of this compound are as follows: ¹H NMR (600 MHz, CDCl₃) δ 7.99-7.86 (m, 4H), 7.87-7.81 (m, 2H), 7.50-7.47 (m, 2H), 7.41-7.38 (m, 1H), 7.37-7.34 (m, 4H), 7.27-7.25 (m, 2H), 6.11 (t, J=9.6 Hz, 1H), 5.63 (t, J=9.6 Hz, 1H), 5.26-5.23 (m, 3H), 5.13 (dd, J=15.0, 9.0 Hz, 1H), 4.99 (dd, J=15.0, 9.0 Hz, 1H), 4.17 (ddd, J=10.8, 4.8, 3.0 Hz, 1H), 3.73 (dd, J=10.8, 3.0 Hz, 1H), 3.60 (dd, J=10.8, 4.8 Hz, 1H), 3.46 (s, 3H), 3.16-3.03 (m, 1H), 2.24-2.23 (m, 1H), 2.18-2.11 (m, 1H), 2.04-1.88 (m, 3H), 1.87-1.82 (m, 1H), 1.78 (dt, J=13.2, 3.0 Hz, 1H), 1.71-1.63 (m, 1H), 1.54-1.37 (m, 9H), 1.24-0.77 (m, 22H), 0.66 (s, 3H). ¹³C NMR (150 MHz, CDCl₃) δ 166.1, 165.4, 141.0, 138.5, 133.5, 133.5, 133.2, 130.2, 130.0, 129.9, 129.6, 129.5, 129.5, 129.4, 128.6, 128.5, 121.8, 97.2, 80.5, 72.4, 70.9, 69.8, 69.2, 67.1, 57.1, 56.2, 55.7, 51.4, 50.4, 42.4, 40.7, 39.9, 39.1, 37.4, 37.1, 32.1, 32.1, 32.1, 29.1, 28.2, 25.6, 24.6, 21.4, 21.3, 21.2, 19.6, 19.2, 12.5, 12.2. HRMS (ESI-TOF) m/z: [M+Na]⁺ calc. for C₅₇H₇₂O₉Na 923.5069, found: 923.5071.

### Example 92

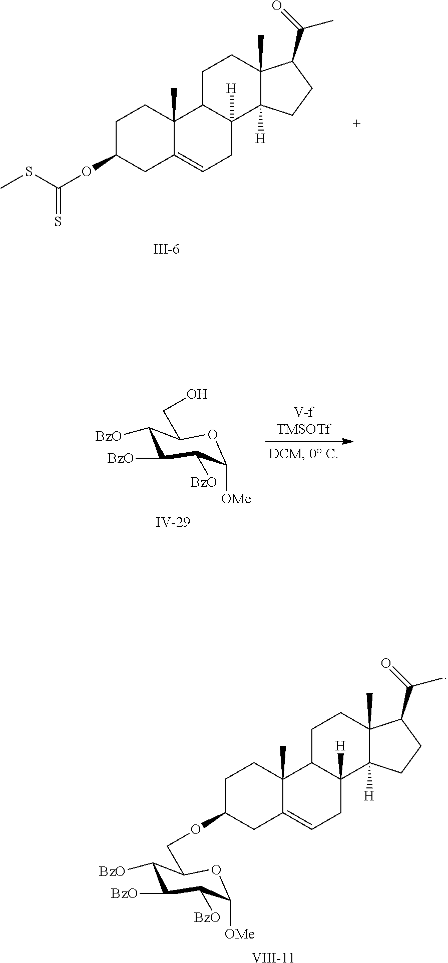

According to the reaction standard operating procedure, pregnenolone xanthate III-6 (18.3 mg, 0.045 mmol, 1.5 equiv) and acceptor IV-29 (15.2 mg, 0.03 mmol, 1.0 equiv), benziodoxole reagent V-f (9.0 mg, 0.012 mmol, 0.4 equiv), and pre-activated molecular sieves were added to the reaction flask. Under argon atmosphere, anhydrous DCM (c=0.1 M, calculated based on the reactant with 1.0 equivalent) was added. The reaction system was pre-cooled to 0° C., TMSOTf (2.2 μL, 0.012 mmol, 0.4 equiv) was added, and the reaction was carried out at 0° C. for 24 hours. After purification by silica gel column chromatography, the target product VIII-11 (16.5 mg, 69%) was obtained. ¹H NMR (600 MHz, CDCl₃) δ 7.96-7.92 (m, 4H), 7.85 (d, J=7.8 Hz, 2H), 7.51-7.47 (m, 2H), 7.44-7.31 (m, 5H), 7.26 (t, J=7.8 Hz, 2H), 6.11 (t, J=9.6 Hz, 1H), 5.63 (t, J=9.6 Hz, 1H), 5.26-5.23 (m, 3H), 4.21-4.10 (m, 1H), 3.73 (dd, J=10.8, 2.4 Hz, 1H), 3.60 (dd, J=10.8, 4.8 Hz, 1H), 3.46 (s, 3H), 3.16-3.05 (m, 1H), 2.49 (t, J=9.0 Hz, 1H), 2.28-2.24 (m, 1H), 2.19-2.12 (m, 2H), 2.09 (s, 3H), 2.02-1.178 (m, 4H), 1.68-1.59 (m, 3H), 1.58-1.36 (m, 6H), 1.28-1.03 (m, 3H), 1.00-0.89 (m, 5H), 0.60 (s, 3H).

Although the present disclosure is described in detail with reference to the foregoing embodiments, a person having ordinary skill in the art can still modify the technical solutions described in the foregoing embodiments or perform equivalent replacements for some of the technical features. Any modification, equivalent replacement, improvement, etc. made within the spirit and principle of the disclosure shall be included in the protection scope of the disclosure.

What is claimed is:

1. A glycosylation method involving a benziodoxole reagent, comprising: activating a glycosyl donor as shown in formula I under a combined action of the benziodoxole reagent having a structural formula as shown in formula V and an acid catalyst and then reacting with a nucleophilic reagent to obtain a glycosylation product as shown in formula VI, with a reaction route as shown in formula 1: 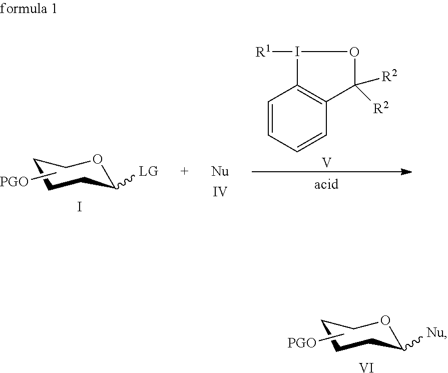
in the glycosyl donor represented by formula I, one or a plurality of hydroxyl groups on a sugar ring are protected by a glycosyl protecting group, and LG is an alkylthio group, an arylthio group, an aryl selenoyl group, a thiocarboxylate group, an o-alkynyl benzoate group, a pentenoate group, a pentynoate group, or a hexynoate group,
in the benziodoxole reagent having a structural formula as shown in formula V, R¹ is chlorine, hydroxyl, trimethylsilyloxy, trifluoromethanesulfonyl ester, or benziodooxolaneoxy, and R² is methyl or trifluoromethyl, and
Nu in formula 1 represents the nucleophilic reagent, and the nucleophilic reagent is selected from primary alcohol, secondary alcohol, or carboxylic acid.

2. The glycosylation method involving the benziodoxole reagent according to claim 1, wherein the glycosyl donor represented by formula I is selected from compounds having any one of the following structures:

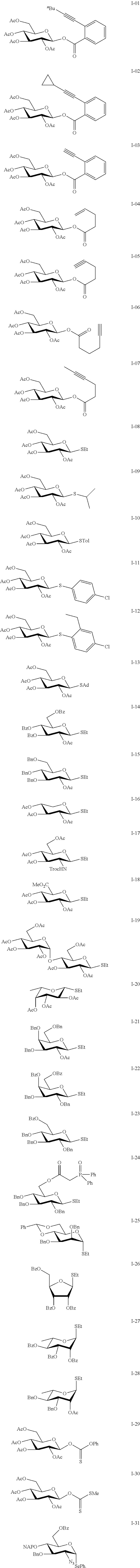

3. The glycosylation method involving the benziodoxole reagent according to claim 1, wherein the benziodoxole reagent having the structure shown in formula V is selected from compounds having any one of the following structures:

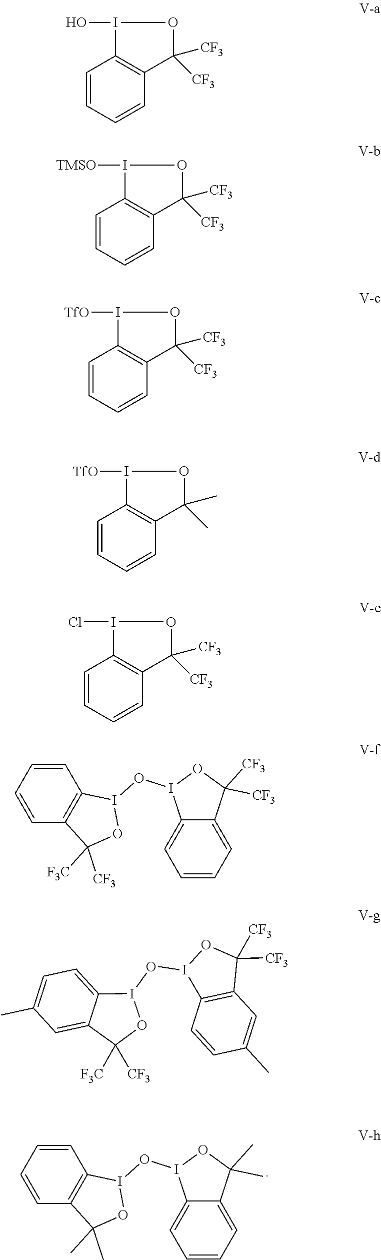

4. The glycosylation method involving the benziodoxole reagent according to claim 1, wherein a molar ratio of the glycosyl donor to the benziodoxole reagent is 1:(0.25 to 0.5).

5. The glycosylation method involving the benziodoxole reagent according to claim 1, wherein the nucleophilic reagent is selected from compounds having any one of the following structures:

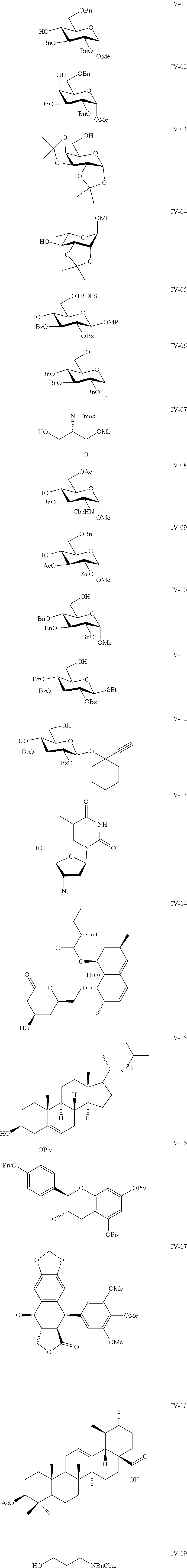

6. An alkylation method involving a benziodoxole reagent, comprising activating an alkylation reagent as shown in formula II under a combined action of the benziodoxole reagent having a structure as shown in formula V and an acid catalyst and then reacting with a nucleophilic reagent to obtain an alkylation product as shown in formula VII, with a reaction route as shown in formula 2; or activating an alkylation reagent as shown in formula III under the combined action of the benziodoxole reagent having a structure as shown in formula V and an acid catalyst and then reacting with a nucleophilic reagent to obtain an alkylation product as shown in formula VIII, with a reaction route as shown in formula 3; 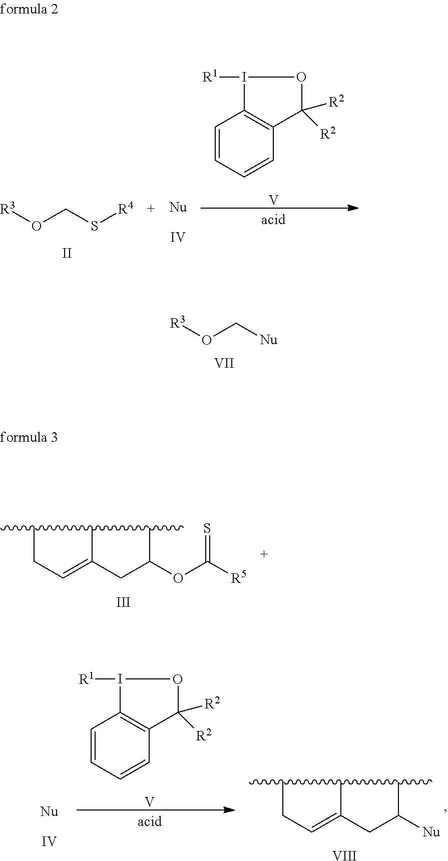
wherein the alkylation reagent represented by formula II is a thioether alkylation reagent, wherein R³ is an alkyl group, and R⁴ is an alkyl group or an aryl group,
the alkylation reagent represented by formula III is a steroid derivative containing a high allyl thiocarbonate structure, wherein the steroid is cholesterol, diosgenin, dehydroepiandrosterone, pregnenolone, β-sitosterol, or stigmasterol, wherein R⁵ is a phenoxy group or a methylthio group,
in the benziodoxole reagent having a structural formula as shown in formula V, R¹ is chlorine, hydroxyl, trimethylsilyloxy, trifluoromethanesulfonyl ester, or benziodooxolaneoxy, and R² is methyl or trifluoromethyl, and
Nu in both formula II and formula III represents the nucleophilic reagent, and the nucleophilic reagent is selected from primary alcohol, secondary alcohol, phenol, or phosphoric acid.

7. The alkylation method involving the benziodoxole reagent according to claim 6, wherein
the alkylation reagent represented by formula II is selected from compounds having any one of the following structures: 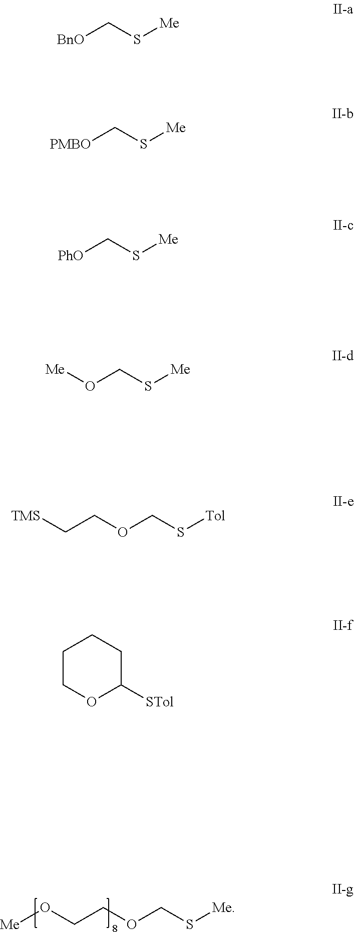
the alkylation reagent represented by formula III is selected from compounds having any one of the following structures:

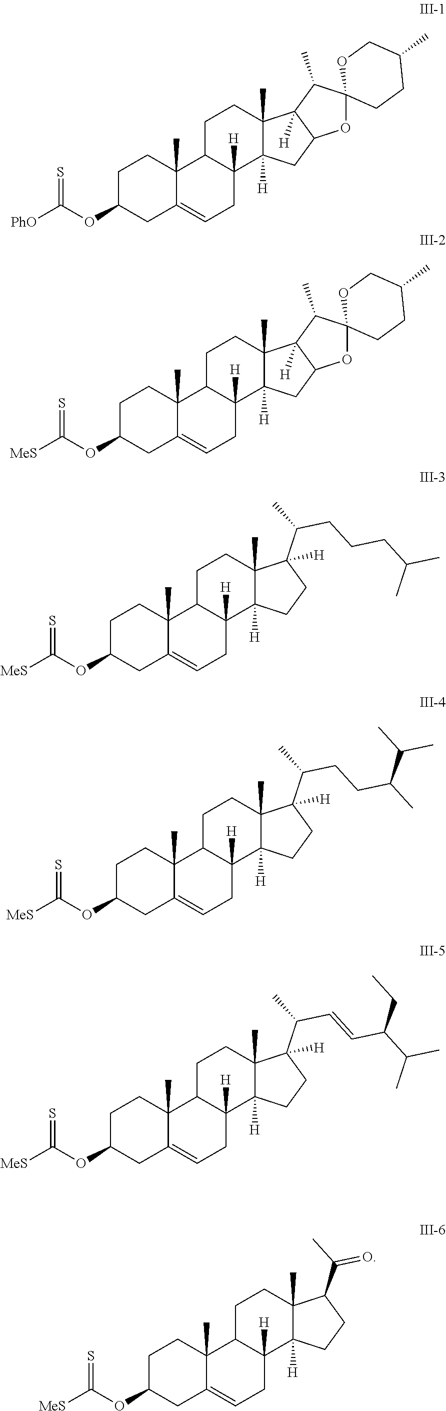

8. The alkylation method involving the benziodoxole reagent according to claim 6, wherein the benziodoxole reagent having the structure shown in formula V is selected from compounds having any one of the following structures:

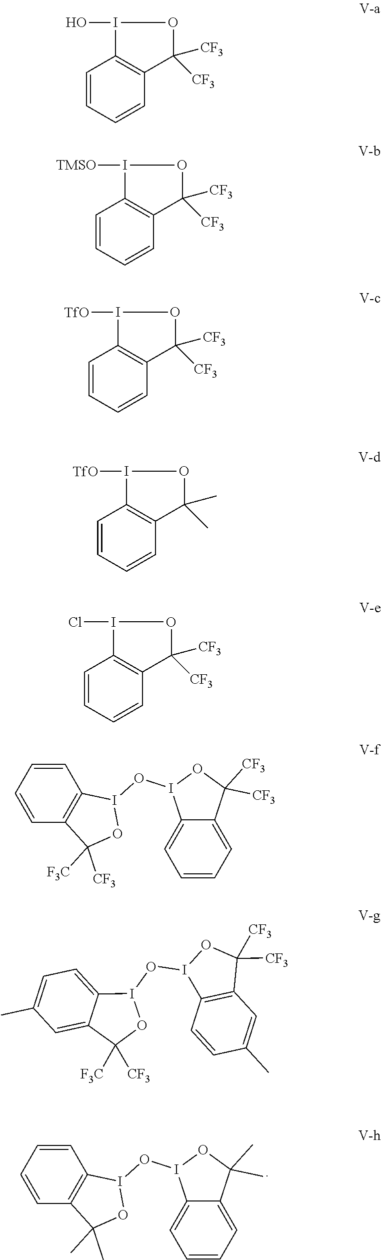

9. The alkylation method involving the benziodoxole reagent according to claim 6, wherein a molar ratio of the alkylation reagent represented by formula II to the benziodoxole reagent is 1: (0.25 to 0.5), and a molar ratio of the alkylation reagent represented by formula III to the benziodoxole reagent is 1:(0.25 to 0.5).

10. The alkylation method involving the benziodoxole reagent according to claim 6, wherein the nucleophilic reagent is selected from compounds having any one of the following structures: [chemical structure]
